(19) United States
(12) Patent Application Publication
(10) Pub. No.: US 2023/0265176 A1
(43) Pub. Date: Aug. 24, 2023

(54) ONE-ARMED ANTIGEN-BINDING MOLECULES AND USES THEREOF

(71) Applicant: Chugai Seiyaku Kabushiki Kaisha, Tokyo (JP)

(72) Inventor: Tomoyuki IGAWA, Tokyo (JP)

(21) Appl. No.: 18/020,656

(22) PCT Filed: Aug. 13, 2021
(86) PCT No.: PCT/JP2021/029787

§ 371 Date: Feb. 10, 2023

(30) Foreign Application Priority Data
Aug. 14, 2020 (JP) 2020-136920

(51) Int. Cl. C07K 16/18

(52) U.S. Cl. CPC C07K 16/18; C07K 2317/52; C07K 2317/55; C07K 2317/622; C07K 2317/51; C07K 2317/515; C07K 2317/734

(57) ABSTRACT

The present disclosure provides an antigen-binding molecule that comprises an antigen-binding moiety which specifically binds to an antigen and an Fc polypeptide comprising a first and second Fc region variants, with the first Fc region variant being fused to the antigen-binding moiety but the second Fc region variant being not fused to the antigen-binding moiety or to any other antigen-binding moieties which bind to the antigen, the antigen-binding molecule having a substantially decreased Fc gamma receptor-binding activity and having a maintained or increased C1q-binding activity when compared to an antigen binding molecule comprising the parent Fc region. The antigen-binding molecule of this format can enhance the clearance of the virus of interest while reducing the risk of ADE.

### TECHNICAL FIELD

The present disclosure relates to one-armed antigen-binding molecules, uses thereof, and such.

### BACKGROUND ART

Antibodies are potent therapeutics as both the Fab and Fc parts of the antibody can be harnessed to neutralize a target. After an antibody binds to its target via the Fab region, the Fc region can recruit molecules such as complement or Fc receptors to further activate the immune system. The target will then be eliminated by mechanisms such as complement-dependent cytotoxicity (CDC), antibody-dependent cellular cytotoxicity (ADCC), and antibody-dependent cellular phagocytosis (ADCP).

Complement-dependent cytotoxicity (CDC) is mediated by the “classical” complement pathway, which is a cascade of enzymatic reactions involving complement proteins C1 through C9. Activation of the classical pathway is first triggered when complement C1q binds to the antibody Fc. Complement protein C1q is a large protein complex consisting of six globular heads and a collagen-like tail, and each globular head is able to interact with an antibody Fc. As the affinity of an individual globular head in C1q for an antibody Fc is weak, C1q binds only weakly to monomeric IgG, and does not activate the classical pathway. This weak affinity is essential for homeostasis as the concentrations of C1q and antibodies in blood are high. However, when a target is densely coated with antibodies, C1q is able to engage multiple Fcs and bind with high avidity, and thus activating the complement pathway. Activation of the classical pathway cascade leads to deposition of proteins such as complement C4b and C3b onto the surface of the target. C4b and C3b mark the target for phagocytic uptake by cells that express complement receptors such as CR1 through CR4 and CRIg. From C3b, the classical pathway cascade also progresses further and results in the deposition of C5b, C6, C7, C8 and C9 proteins, which assemble to become a pore-forming C5b-9 membrane-attack complex (MAC). The formation of MAC on the target surface disrupts the membrane integrity and ultimately leads to target lysis. Antibody-mediated CDC activity has been long known for its ability to mediate the killing of bacteria, and was discovered in 1895 by Jules Bordet. More recently, antibody mediated CDC activity has also been described to mediate the clearance of several different types of viruses (NPL 1).

Antibody-dependent cellular cytotoxicity (ADCC) and antibody-dependent cellular phagocytosis (ADCP) are mediated by interactions between the antibody Fc and cells expressing Fc gamma receptors. In ADCC, effector cytotoxic cells such as natural killer cells recognize antibody-bound targets and release lytic enzymes to destroy the target. In ADCP, phagocytes such as macrophages, monocytes, and neutrophils take up the antibody opsonized targets and clear them from the circulation. Although ADCP is an important process for protective immunity, some pathogens exploit the ability of ADCP to enhance their infectivity in a process known as antibody-dependent enhancement (ADE). ADE occurs when antibodies enhance pathogen entry into host cells via Fc gamma receptors. This occurs when antibody titres are insufficient to neutralize the pathogen, or when the antibodies against the pathogen are non-neutralizing in nature. To avoid therapeutically administered antibodies from having ADE function, mutations are introduced into the Fc region to reduce or silence Fc gamma receptor binding functions. Alternatively, Fc subclasses with naturally weak Fc gamma receptor binding function such as IgG2 and IgG4 are used.

### CITATION LIST

### Non Patent Literature

NPL 1 Front Microbiol. 2017; 8: 1117

### SUMMARY OF INVENTION

### Technical Problem

The present inventors have thought that it is challenging to design a one-armed antigen-binding molecule having substantially decreased Fc gamma receptor binding and maintained or increased complement C1q-binding activity compared with a conventional two-armed, bivalent antibody. Generating such a molecule is one of potential strategy to reduce the risk of antibody-dependent enhancement (ADE) in various virus related diseases, caused by the virus entry into host cells via Fc gamma receptors, while having enough clearance of the virus of interest. The present invention has been made on the basis of such an idea. An objective of the present disclosure is to provide antigen-binding molecules that can increase the clearance of the virus of interest while reducing the risk of ADE, methods for producing the antigen-binding molecules, and pharmaceutical compositions comprising such an antigen-binding molecule as an active ingredient.

### Solution to Problem

The inventors found that an antigen-binding molecule that comprises an antigen-binding moiety which specifically binds to an antigen and an Fc polypeptide comprising a first and second Fc region variants, with the first Fc region variant being fused to the antigen-binding moiety but the second Fc region variant being not fused to the antigen-binding moiety or to any other antigen-binding moieties which bind to the antigen, can cause an increase of the clearance of the virus of interest. Furthermore, the inventors found that the antigen-binding molecule mentioned above modified such that the antigen-binding molecule has a substantially decreased Fc gamma receptor-binding activity and has a maintained or increased C1q-binding activity when compared to an antigen binding molecule comprising the parent Fc region can increase the clearance of the virus of interest while reducing the risk of ADE.

More specifically, the present invention provides the following:
[1] An antigen-binding molecule comprising:
   (i) a first antigen-binding moiety which specifically binds to an antigen, and
   (ii) an Fc polypeptide,
      wherein the Fc polypeptide comprises a first Fc region variant and a second Fc region variant each comprising at least one amino acid alteration relative to a parent Fc region, wherein the first Fc region variant is fused to the first antigen-binding moiety, provided that the second Fc region variant is not fused to any other antigen-binding moieties which specifically binds to the antigen, and wherein the antigen-binding molecule has a substantially decreased Fc gamma R-binding activity and has a maintained or increased C1q-binding activity when compared to an antigen binding molecule comprising the parent Fc region.
[2] The antigen-binding molecule of [1], wherein the antigen-binding molecule further comprises a second antigen-binding moiety which specifically binds to an epitope on the antigen that is different from the epitope on the antigen bound by the first antigen-binding moiety.
[3] The antigen-binding molecule of [1], wherein the antigen-binding molecule further comprises a second antigen-binding moiety which specifically binds to the same epitope as the one on the antigen bound by the first antigen-binding moiety.
[4] The antigen-binding molecule of [2] or [3], wherein the second antigen-binding moiety is fused to the N-terminus of the first antigen-binding moiety.
[5] The antigen-binding molecule of any one of [1] to [4], wherein the first antigen-binding moiety and/or the second antigen-binding moiety comprises a Fab, scFv, VHH, VL, VH, single domain antibody or ligand.
[6] The antigen-binding molecule of [5], wherein each of the first antigen-binding moiety and the second antigen-binding moiety comprises a Fab.
[7] The antigen-binding molecule of any one of [1] to [6], wherein each of the first Fc region variant and the second Fc region variant comprises Ala at position 234 and Ala at position 235 according to EU numbering.
[8] The antigen-binding molecule of [7], wherein each of the first Fc region variant and the second Fc region variant comprises further amino acid alterations at positions of any one of the following (a)-(c):
   (a) positions 267, 268, and 324;
   (b) positions 236, 267, 268, 324, and 332; and
   (c) positions 326 and 333;
   according to EU numbering.
[9] The antigen-binding molecule of [8], wherein each of the first Fc region variant and the second Fc region variant comprises amino acids selected from the group consisting of:
   (a) Glu at position 267;
   (b) Phe at position 268;
   (c) Thr at position 324;
   (d) Ala at position 236;
   (e) Glu at position 332;
   (f) Ala, Asp, Glu, Met, or Trp at position 326; and
   (g) Ser at position 333;
   according to EU numbering.
[10] The antigen-binding molecule of any one of [1] to [9], wherein each of the first Fc region variant and the second Fc region variant comprises amino acids selected from the group consisting of:
   (a) Ala at position 434;
   (b) Ala at position 434, Thr at position 436, Arg at position 438, and Glu at position 440;
   (c) Leu at position 428, Ala at position 434, Thr at position 436, Arg at position 438, and Glu at position 440;
   (d) Leu at position 428, and Ala at position 434; and
   (e) Leu at position 428, Ala at position 434, Arg at position 438, and Glu at position 440;
   according to EU numbering.
[11] The antigen-binding molecule of any one of [1] to [10], wherein each of the first Fc region variant and the second Fc region variant comprises at least one amino acid alteration that enhances hexamer formation.
[12] The antigen-binding molecule of any one of [1] to [11], wherein each of the first Fc region variant and the second Fc region variant comprises at least one amino acid alteration promoting the association of the first Fc region variant and the second Fc region variant.
[13] The antigen-binding molecule of any one of [1] to [12], which has reduced risk to cause antibody-dependent enhancement (ADE) of entry of a pathogen expressing the antigen into a cell.
[14] The antigen-binding molecule of any one of [1] to [13], which recruits C1q to eliminate a pathogen or a cell infected by the pathogen via complement-dependent cytotoxicity (CDC).
[15] The antigen-binding molecule of [13] or [14], wherein the pathogen is a virus.
[16] A pharmaceutical composition comprising the antigen-binding molecule of any one of [1] to [15], and a pharmaceutically acceptable carrier.
[16-1] A pharmaceutical composition comprising two or more of the antigen-binding molecules of any one of [1] to [15] which are different from each other, and a pharmaceutically acceptable carrier.
[16-2] A pharmaceutical composition comprising the antigen-binding molecule of any one of [1] to [15] as a first antigen-binding molecule, for use in combination with a second antigen-binding molecule which is selected from any one of [1] to [15] and is different from the first antigen-binding molecule.
[16-3] The pharmaceutical composition of [16-2], wherein the second antigen-binding molecule binds to an epitope on the antigen that is different from the epitope on the antigen bound by the first antigen-binding molecule.
[16-4] The pharmaceutical composition of [16-2] or [16-3], wherein the first antigen-binding molecule is administered simultaneously with the second antigen-binding molecule.
[16-5] The pharmaceutical composition of [16-2] or [16-3], wherein the first antigen-binding molecule is administered before or after administration of the second antigen-binding molecule.
[17] An isolated nucleic acid encoding the antigen-binding molecule of any one of [1] to [15].
[18 ] A vector comprising the nucleic acid of [17].
[19 ] A host cell comprising the nucleic acid of [17] or the vector of [18].
[20 ] A method of producing the antigen-binding molecule of any one of [1] to [15], comprising culturing the host cell of [19].
[21 ] The antigen-binding molecule of any one of [1] to [15] or the pharmaceutical composition of [16] for use in treatment of a virus infectious disease.
[22] A method for treatment of a virus-infectious-disease subject who has been infected with a virus, comprising administering to the subject the antigen-binding molecule of any one of [1] to [15].
[23 ] Use of the antigen-binding molecule of any one of [1] to [15] for the manufacture of a medicament for treatment of a virus infectious disease.
[24] A method for producing an antigen-binding molecule, which comprises the steps of:
   (a) selecting an antigen-binding molecule that comprises an Fc polypeptide comprising a first Fc region variant and a second Fc region variant, each comprising at least one amino acid alteration relative to a parent Fc region, and has a substantially decreased Fc gamma receptor-binding activity and has a maintained or increased C1q-binding activity when compared to an antigen binding molecule comprising the parent Fc region;
   (b) obtaining a gene encoding an antigen-binding molecule in which the first Fc region variant of the antigen-binding molecule selected in (a) is fused to a first antigen-binding moiety which specifically binds to an antigen and the second Fc region variant of the antigen-binding molecule selected in (a) is not fused to any other antigen-binding moiety which specifically binds to the antigen; and
   (c) producing an antigen-binding molecule using the gene obtained in (b).
[25] The method of [24], wherein the first antigen-binding moiety in the antigen-binding molecule encoded by the gene obtained in (b) carries a second antigen-binding moiety fused thereto which specifically binds to an epitope on the antigen that is different from the epitope on the antigen bound by the first antigen-binding moiety.
[26] The method of [24], wherein the first antigen-binding moiety in the antigen-binding molecule encoded by the gene obtained in (b) carries a second antigen-binding moiety fused thereto which specifically binds to the same epitope as the one on the antigen bound by the first antigen-binding moiety.
[27] An antigen-binding molecule produced by the production method of any one of [24] to [26].
[28] A method for screening for an antigen-binding molecule, which comprises the steps of:
   (a) selecting an antigen-binding molecule that comprises an Fc polypeptide comprising a first Fc region variant and a second Fc region variant, each comprising at least one amino acid alteration relative to a parent Fc region, and has a substantially decreased Fc gamma receptor-binding activity and has a maintained or increased C1q-binding activity when compared to an antigen binding molecule comprising the parent Fc region;
   (b) obtaining a gene encoding an antigen-binding molecule in which the first Fc region variant of the antigen-binding molecule selected in (a) is fused to a first antigen-binding moiety which specifically binds to an antigen and the second Fc region variant of the antigen-binding molecule selected in (a) is not fused to any other antigen-binding moiety which specifically binds to the antigen; and
   (c) producing an antigen-binding molecule using the gene obtained in (b).
[29] The method of [28], wherein the first antigen-binding moiety in the antigen-binding molecule encoded by the gene obtained in (b) carries a second antigen-binding moiety fused thereto which specifically binds to an epitope on the antigen that is different from the epitope on the antigen bound by the first antigen-binding moiety.
[30] The method of [28], wherein the first antigen-binding moiety in the antigen-binding molecule encoded by the gene obtained in (b) carries a second antigen-binding moiety fused thereto which specifically binds to the same epitope as the one on the antigen bound by the first antigen-binding moiety.
[31] An antigen-binding molecule produced by the production method of any one of [28] to [30].

### BRIEF DESCRIPTION OF DRAWINGS

FIG. 1 is a graph showing the percentage of cells lysed by antibody-mediated CDC using antibodies of the conventional two-arm format (bivalent anti-HER2-SG1095, bivalent anti-HER2-SG1408, and bivalent anti-HER2-SG192, referred to as 2arm-SG1095, 2arm-SG1408, and 2arm-SG192 respectively in the Figure) and antibodies of the one-arm format (one-arm anti-HER2-SG1095, one-arm anti-HER2-SG1048, and one-arm anti-HER2-SG192, referred to as 1arm-SG1095, 1arm-SG1408, and 1arm-SG192 respectively in the Figure).

FIG. 2 is a graph showing the percentage of cells lysed by antibody-mediated CDC using antibodies of the conventional two-arm format (bivalent anti-HER2-SG1095, bivalent anti-HER2-SG1095R, bivalent anti-HER2-SG1095RG, bivalent anti-HER2-SG1095RGY, bivalent anti-HER2-SG1095ER, bivalent anti-HER2-SG1408, and bivalent anti-HER2-SG192, referred to as 2arm-SG1095, 2arm-SG1095R, 2arm-SG1095RG, 2arm-SG1095RGY, 2arm-SG1095ER, 2arm-SG1408, and 2arm-SG192 respectively in the Figure).

FIG. 3 is a graph showing the percentage of cells lysed by antibody-mediated CDC using antibodies of the one-arm format (one-arm anti-HER2-SG1095, one-arm anti-HER2-SG1095R, one-arm anti-HER2-SG1095RG, one-arm anti-HER2-SG1095RGY, one-arm anti-HER2-SG1095ER, one-arm anti-HER2-SG1048, and one-arm anti-HER2-SG192, referred to as 1arm-SG1095, 1arm-SG1095R, 1arm-SG1095RG, 1arm-SG1905RGY, 1arm-SG1095ER, 1arm-SG1408, and 1arm-SG192 respectively in the Figure).

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

The definitions and detailed description below are provided to facilitate understanding of the present disclosure illustrated herein.

### Definitions

### Amino Acids

Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Alteration of Amino Acids

Amino acid alteration means any of substitution, deletion, addition, and insertion, or a combination thereof. In the present disclosure, amino acid alteration may be rephrased as amino acid mutation or amino acid modification. For amino acid alteration in the amino acid sequence of an antigen-binding molecule, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is suitable to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid alteration includes every combination where “and” and “or” are suitably combined. Specifically, for example, “the amino acids at positions 33, 55, and/or 96 are substituted” includes the following variation of amino acid alterations amino acid(s) at (a) position 33, (b) position 55, (c) position 96, (d) positions 33 and 55, (e) positions 33 and 96, (f) positions 55 and 96, and (g) positions 33, 55, and 96.

Furthermore, herein, as an expression showing alteration of amino acids, an expression that shows, at the left and the right of a number indicating a specific position, one-letter or three-letter codes for amino acids before and after alteration, respectively, may be used appropriately. For example, the alteration N100bL or Asn100bLeu used when substituting an amino acid contained in an antibody variable region indicates substitution of Asn at position 100b (according to Kabat numbering) with Leu. That is, the number shows the amino acid position according to Kabat numbering, the one-letter or three-letter amino-acid code written before the number (at the left of the number) shows the amino acid before substitution, and the one-letter or three-letter amino-acid code written after the number (at the right of the number) shows the amino acid after substitution. Similarly, the alteration P238D or Pro238Asp used when substituting an amino acid of the Fc region contained in an antibody constant region indicates substitution of Pro at position 238 (according to EU numbering) with Asp. That is, the number shows the amino acid position according to EU numbering, the one-letter or three-letter amino-acid code written before the number (at the left of the number) shows the amino acid before substitution, and the one-letter or three-letter amino-acid code written after the number (at the right of the number) shows the amino acid after substitution.

### Polypeptides

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

### Percent (%) Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and
where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Recombinant Methods and Compositions

Antibodies and antigen-binding molecules may be produced using recombinant methods and compositions, e.g., as described in U.S. Pat. No. 4,816,567. In one embodiment, isolated nucleic acid encoding an antibody as described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp2/0 cell). In one embodiment, a method of making the antigen-binding molecule of the present disclosure is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody described herein, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Recombinant production of an antigen-binding molecule described herein could be done with methods similar to those described above, by using a host cell comprises (e.g., has been transformed with) one or plural vectors comprising nucleic acid that encodes an amino acid sequence comprising the whole antigen-binding molecule or part of the antigen-binding molecule.

### Antigen-Binding Molecules

The term “antigen-binding molecule”, as used herein, refers to any molecule that comprises an antigen-binding site, antigen-binding moiety, or any molecule that has binding activity to an antigen, and may further refer to molecules such as a peptide or protein having a length of about five amino acids or more. The peptide and protein are not limited to those derived from a living organism, and for example, they may be a polypeptide produced from an artificially designed sequence. They may also be any naturally-occurring polypeptide, synthetic polypeptide, recombinant polypeptide, and such. Scaffold molecules comprising a known stable conformational structure such as alpha/beta barrel as scaffold, and in which part of the molecule is made into an antigen-binding site, is also one embodiment of the antigen binding molecule described herein.

In one aspect, an antigen-binding molecule of the present disclosure can be a “one-armed (or one-arm) antigen-binding molecule”. In one aspect, the “one-armed antigen-binding molecule” refers to an antigen-binding molecule that specifically binds to an antigen through an antigen-binding site, an antigen-binding moiety, or any molecules having binding activity to the antigen fused to a first Fc region variant comprised in an Fc polypeptide of the “one-armed antigen-binding molecule”, the Fc polypeptide comprising the first Fc region variant and second Fc region variant. In a certain aspect, the second Fc region variant comprised in the Fc polypeptide of the “one-armed antigen-binding molecule” is not fused to any other antigen-binding sites, antigen-binding moieties etc., which can bind to the same antigen to which the first antigen binding site, the first antigen binding moiety, etc. mentioned above binds and/or to any other antigen-binding sites, antigen-binding moieties etc., which can bind to different antigen (or epitope) from the one to which the first antigen binding site, the first antigen binding moiety, etc. mentioned above binds. The “one-armed” antigen-binding molecule provided herein can show enhanced hexamer formation as compared to conventional two-armed antigen-binding molecule, which can enhance binding to complement C1q.

In contrast, a “two-armed (or two-arm) antigen-binding molecule” refers to an antigen-binding molecule that specifically binds to an antigen through two antigen-binding sites, two antigen-binding moieties, or any two molecules having binding activity to the antigen. One and the other of the two antigen-binding sites, two antigen-binding moieties, etc. are fused to a first Fc region and a second Fc region, respectively, comprised in an Fc polypeptide of the “two-armed antigen-binding molecule”, unless otherwise indicated. In one non-limiting embodiment, IgG-type conventional bivalent antibodies can be examples of the “two-armed antigen-binding molecules”.

As used herein, an antigen-binding molecule containing at least two antigen-binding moieties (or antigen-binding domains), wherein at least one of the antigen-binding moieties binds to a first epitope in an antigen molecule and at least another one of the antigen-binding moieties binds to a second epitope in the antigen molecule, is called a multispecific antigen-binding molecule from the standpoint of its reaction specificity.

When a single antigen-binding molecule binds to two different epitopes through two types of antigen-binding moieties contained in the antigen-binding molecule, this antigen-binding molecule is called “a bispecific antigen-binding molecule”. When a single antigen-binding molecule binds to three different epitopes through three types of antigen-binding domains contained in the antigen-binding molecule, this antigen-binding molecule is called “a trispecific antigen-binding molecule”.

In one embodiment, the paratope in an antigen-binding moiety that binds to a first epitope in the antigen molecule has a different structure from that of the paratope in an antigen-binding moiety that binds to a second epitope that is structurally different from the first epitope. Therefore, an antigen-binding molecule containing at least two antigen-binding moieties (or domains), wherein at least one of the antigen-binding moieties binds to a first epitope in an antigen molecule and at least another one of the antigen-binding moieties binds to a second epitope in the antigen molecule, is called “a multiparatopic antigen-binding molecule” from the standpoint of its structure and specificity.

When a single antigen-binding molecule binds to two different epitopes through two types of antigen-binding moieties contained in the antigen-binding molecule, this antigen-binding molecule is called “a biparatopic antigen-binding molecule”. When a single antigen-binding molecule binds to three different epitopes through three types of antigen-binding moieties contained in the antigen-binding molecule, this antigen-binding molecule is called “a triparatopic antigen-binding molecule”.

Multivalent multispecific or multiparatopic antigen-binding molecules comprising one or several antigen-binding moieties and methods for preparing them have been described in Non-Patent Documents such as Conrath et al. (J. Biol. Chem. (2001) 276 (10) 7346-7350), Muyldermans (Rev. Mol. Biotech. (2001) 74, 277-302), and Kontermann R E ((2011) Bispecific Antibodies (Springer-Verlag)) and in Patent Documents such as WO 1996/034103 and WO 1999/023221. Antigen-binding molecules of the present invention can be produced using the multispecific or multiparatopic antigen-binding molecules and methods for preparing them described in these documents.

In one aspect, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region,
wherein the first Fc variant region is fused to the first antigen-binding moiety, provided that the second Fc region variant is not fused to the first antigen-binding moiety or to any other antigen-binding moieties which specifically binds to the antigen.

In one embodiment, the antigen-binding molecule of the present disclosure is a biparatopic antigen-binding molecule (or one-armed biparatopic antigen-binding molecule), i.e. specifically binding to two different epitopes of the antigen of interest through two antigen binding moieties contained in the antigen-binding molecule.

In some embodiments, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen. In one of such embodiments, the antigen-binding molecule of the present disclosure further comprises a second antigen-binding moiety which specifically binds to an epitope on the antigen that is different from the epitope on the antigen bound by the first antigen-binding moiety.

In some embodiments, the antigen-binding molecule of the present application is a bivalent antigen-binding molecule (one-armed bivarent antigen-binding molecule), wherein the antigen-binding molecule comprises a first and second antigen-binding moiety which specifically binds to the same epitope on the antigen.

In some embodiments, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen. In one of such embodiments, the antigen-binding molecule of the present disclosure further comprises a second antigen-binding moiety which specifically binds to the same epitope on the antigen as the epitope on the antigen bound by the first antigen-binding moiety.

In one aspect, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen, and wherein the antigen-binding molecule has a substantially decreased Fc gamma receptor-binding activity and has a maintained or increased C1q-binding activity when compared to an antigen binding molecule comprising the parent Fc region.

In one embodiment, the antigen-binding molecule of the present disclosure is a biparatopic antigen-binding molecule (or one-armed biparatopic antigen-binding molecule), i.e. specifically binding to two different epitopes of the antigen of interest through two antigen binding moieties contained in the antigen-binding molecule.

In some embodiments, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen, wherein the antigen-binding molecule has a substantially decreased Fc gamma receptor-binding activity and has a maintained or increased C1q-binding activity when compared to an antigen binding molecule comprising the parent Fc region. In one of such embodiments, the antigen-binding molecule of the present disclosure further comprises a second antigen-binding moiety which specifically binds to an epitope on the antigen that is different from the epitope on the antigen bound by the first antigen-binding moiety.

In some embodiments, the antigen-binding molecule of the present application is a bivalent antigen-binding molecule (one-armed bivarent antigen-binding molecule), wherein the antigen-binding molecule comprises a first and second antigen-binding moiety which specifically binds to the same epitope on the antigen.

In some embodiments, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen, wherein the antigen-binding molecule has a substantially decreased Fc gamma receptor-binding activity and has a maintained or increased C1q-binding activity when compared to an antigen binding molecule comprising the parent Fc region. In one of such embodiments, the antigen-binding molecule of the present disclosure further comprises a second antigen-binding moiety which specifically binds to the same epitope on the antigen as the epitope on the antigen bound by the first antigen-binding moiety.

In one aspect, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen, and wherein the first and second variant Fc regions comprise at least one amino acid alteration that enhances hexamer formation. In one embodiment, the antigen-binding molecule of the present disclosure has a substantially decreased Fc gamma R-binding activity. In one embodiment, the antigen-binding molecule of the present disclosure has a maintained (not substantially decreased) or increased C1q-binding activity. In one embodiment, the antigen-binding molecule of the present disclosure has a substantially decreased Fc gamma R-binding activity and has a maintained (not substantially decreased) or increased C1q-binding activity.

In some embodiments, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc region variant is fused to the first antigen-binding moiety, provided that the second Fc region variant is not fused to the first antigen-binding moiety or to any other antigen-binding moieties which specifically binds to the antigen, and wherein each of the first Fc region variant and the second Fc region variant comprises Ala at position 234 and Ala at position 235 according to EU numbering, and at least one amino acid alteration of at least one position selected from the group consisting of: 236, 267, 268, 324, 326, 332, and 333, according to EU numbering. Alternatively, the first and/or second Fc region variants may comprise any one or more of the amino acid alterations described in Table 1. In one of such embodiments, the antigen-binding molecule has a substantially decreased Fc gamma receptor-binding activity and has a maintained or increased C1 q-binding activity when compared to an antigen binding molecule comprising the parent Fc region which does not have the above-mentioned amino acid alteration(s). In one embodiment, the antigen-binding molecule further comprises a second antigen-binding moiety which specifically binds to an epitope on the antigen that is different from the epitope on the antigen bound by the first antigen-binding moiety. In another embodiment, the antigen-binding molecule further comprises a second antigen-binding moiety which specifically binds to the same epitope on the antigen as the epitope on the antigen bound by the first antigen-binding moiety.

In some embodiments, the first antigen-binding moiety and/or the second antigen-binding moiety of the present disclosure comprises a Fab, scFv, VHH, VL, VH, single domain antibody, or ligand. In a more specific embodiment, the first antigen-binding moiety and the second antigen-binding moiety of the present disclosure comprises a Fab domain.

According to any of the above embodiments, components of the antigen-binding molecules (e.g. antigen binding moiety, Fc polypeptide, the first Fc region (or “Fc domain”), the second Fc region) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, (G₄S)ₙ or G₄(SG₄)ₙ peptide linkers, wherein n is generally a number between 1 and 10, typically between 2 and 4. For example, the first antigen-binding moiety may be fused to the N-terminus of the first Fc region variant directly or through a suitable linker. The second antigen-binding moiety may be fused to the N-terminus of the first antigen-binding moiety directly or through a suitable linker.

In the biparatopic or bispecific antigen-binding molecule of the present disclosure having a first and a second antigen-binding moieties, the first and the second antigen-binding moieties may be (directly or indirectly) fused to the same first Fc region variant thereby providing an antigen-binding molecule in the “one-armed” molecular format, or the first antigen-binding moiety may be fused to one of the first and the second Fc region variant and the second antigen-binding moiety may be fused to the other thereby providing an antigen-binding molecule in the conventional “two-armed” molecular format. In one embodiment of any of the one-armed or two-armed biparatopic or bispecific antigen-binding molecules described above comprising a first antigen-binding moiety and a second antigen-binding moiety, the second antigen-binding moiety is capable of binding to a different epitope on the same antigen to which the first antigen-binding moiety binds or on a different antigen from the one to which the first antigen-binding moiety binds.

### Pyroglutamylation

It is known that when an antibody is expressed in cells, the antibody is modified after translation. Examples of the posttranslational modification include cleavage of lysine at the C terminal of the heavy chain by a carboxypeptidase; modification of glutamine or glutamic acid at the N terminal of the heavy chain and the light chain to pyroglutamic acid by pyroglutamylation; glycosylation; oxidation; deamidation; and glycation, and it is known that such posttranslational modifications occur in various antibodies (Journal of Pharmaceutical Sciences, 2008, Vol. 97, p. 2426-2447).

The antigen-binding molecules of the present disclosure also include an antibody which has undergone posttranslational modification. Examples of the antigen-binding molecules of the present disclosure which undergoes posttranslational modification include antibodies which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

Antigen Binding Moiety which Specifically Binds to an Antigen

As used herein, the term “antigen binding moiety” refers to a polypeptide molecule that specifically binds to an antigen. In one embodiment, an antigen binding moiety is able to direct the entity to which it is attached to a target site. Antigen binding moieties may include antibodies, fragments thereof, or ligands, as further defined herein.

In certain embodiments, an antigen binding moiety may include an antigen binding domain or an antibody variable region of an antibody, comprising an antibody heavy chain variable region and an antibody light chain variable region. In certain embodiments, an antigen binding moiety may comprise an antibody constant region as further defined herein and known in the art. Useful heavy chain constant regions include any of the five isotypes: alpha, delta, epsilon, gamma, or mu. Useful light chain constant regions include any of the two isotypes: kappa and lambda.

As used herein, the terms “first”, “second”, and “third” with respect to antigen binding moieties etc., are used for convenience of distinguishing when there is more than one of each type of moiety and such. Use of these terms is not intended to confer a specific order or orientation of the antigen-binding molecule unless explicitly so stated.

In certain embodiments, the first antigen-binding moiety of the present disclosure is generally a Fab molecule, particularly a conventional Fab molecule. In certain embodiments, the antigen-binding moiety (“first antigen-binding moiety”) is “single chain Fv (scFv)”, “single chain antibody”, “Fv”, “single chain Fv 2 (scFv2)”, “Fab”, “F(ab′)2”, VHH, VL, VH, single domain antibody, or any antibody fragment.

In certain embodiments, the second antigen-binding moiety is generally a Fab molecule, particularly a conventional Fab molecule. In certain embodiments, the antigen-binding moiety (“first antigen-binding moiety”) is “single chain Fv (scFv)”, “single chain antibody”, “Fv”, “single chain Fv 2 (scFv₂)”, “Fab”, “F(ab′)₂”, VHH, VL, VH, single domain antibody, or any antibody fragment.

In one non-limiting embodiment, the first antigen-binding moiety and the second antigen-binding moiety of the present disclosure comprise a Fab molecule, particularly a conventional Fab molecule.

In another non-limiting embodiment, in the case that the antigen-binding molecule of the present disclosure further comprises a third or more antigen-binding moiety in its structure, the third (or more) antigen-binding moiety is generally a Fab molecule, particularly a conventional Fab molecule. In a further non-limiting embodiment, the third (or more) antigen-binding moiety is “single chain Fv (scFv)”, “single chain antibody”, “Fv”, “single chain Fv 2 (scFv₂)”, “Fab”, “F(ab′)₂”, VHH, VL, VH, single domain antibody, or any antibody fragment.

In certain embodiments, the antigen-binding moiety of the present disclosure specifically binds to the whole or a portion of a partial peptide of an antigen. Examples of antigens that can be bound by the antigen-binding moiety of the present disclosure include, but are not limited to, ligands (cytokines, chemokines, and such), receptors, cancer antigens, viral antigens, MHC antigens, differentiation antigens, immunoglobulins, and immune complexes partly containing immunoglobulins. In a particular embodiment, the antigen is human antigen or cynomolgus antigen or mouse antigen, most particularly human antigen. In a particular embodiment, the antigen-binding moiety is cross-reactive for (i.e. specifically binds to) human and cynomolgus antigen. In another embodiment, the antigen is an antigen of a pathogenic entities, such as pathogenic microorganisms including viruses, bacteria, mycobacteria, fungi, protozoa, etc. In one non-limiting embodiment, the antigen is a virus or a part thereof which can cause the risk of Antibody-Dependent Enhancement (ADE) in the presence of an antigen-binding molecule binding thereto.

In one aspect, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen, and wherein the antigen-binding molecule has a substantially decreased Fc gamma receptor-binding activity and has a maintained or increased C1q-binding activity when compared to an antigen binding molecule comprising the parent Fc region.

In specific embodiments, the first antigen-binding moiety of the present disclosure specifically binds to HER2, and the HER2 antigen-binding moiety (“first antigen-binding moiety”) comprises the combinations of H-chain CDR 1, CDR 2, and CDR 3 and L-chain CDR 1, CDR 2, and CDR 3 of (b1) below:
(b1) a heavy chain variable region comprising the complementarity determining region (CDR) 1, the CDR 2, and the CDR 3 comprised in the amino acid sequence of SEQ ID NO: 8, and a light chain variable region comprising the CDR 1, the CDR 2, and the CDR 3 comprised in the amino acid sequence of SEQ ID NO: 9.

In specific embodiments, the HER2 antigen-binding moiety (“first antigen-binding moiety”) comprises the antibody variable regions that comprise human antibody frameworks or humanized antibody frameworks.

In specific embodiments, the first antigen-binding moiety comprises (dl) below:
(d1) a heavy chain variable region comprising the amino acid sequence of SEQ ID NO: 8, and a light chain variable region comprising the amino acid sequence of SEQ ID NO: 9.

In one embodiment, the HER2 antigen-binding moiety (“first antigen-binding moiety”) comprises a heavy chain variable region sequence that is at least about 95%, 96%, 97%, 98%, 99% or 100% identical to the amino acid sequence of SEQ ID NO: 8 and a light chain variable region sequence that is at least about 95%, 96%, 97%, 98%, 99% or 100% identical to the amino acid sequence of SEQ ID NO: 9.

In specific embodiments, an antigen-binding molecule of the present invention comprises:
the amino acid sequence of SEQ ID NOs: 8 and 17 (“chain 1” which comprises a variable heavy chain domain (VH) comprising the amino acid sequence of SEQ ID NO: 8 and a constant heavy chain domain 1 (CH1) (site-specific binding domain) and an Fc region comprising the amino acid sequence of SEQ ID NO: 17);
the amino acid sequence of SEQ ID NOs: 9 and 10 (“Chain 2” which comprises a variable the amino acid sequence of SEQ ID NO: 24 (“Chain 3” which comprises an Fc region).

In specific embodiments, an antigen-binding molecule of the present invention comprises:
the amino acid sequence of SEQ ID NOs: 8 and 11 (“chain 1” which comprises a variable heavy chain domain (VH) comprising the amino acid sequence of SEQ ID NO: 8 and a constant heavy chain domain 1 (CH1) (site-specific binding domain) and an Fc region comprising the amino acid sequence of SEQ ID NO: 11);
the amino acid sequence of SEQ ID NOs: 9 and 10 (“Chain 2” which comprises a variable light chain domain (VL) (site-specific binding domain) comprising the amino acid sequence of SEQ ID NO: 9 and a constant light chain domain (CL) comprising the amino acid sequence of SEQ ID NO: 10); and
the amino acid sequence of SEQ ID NO: 18 (“Chain 3” which comprises an Fc region).

In specific embodiments, an antigen-binding molecule of the present invention comprises:
the amino acid sequence of SEQ ID NOs: 8 and 12 (“chain 1” which comprises a variable heavy chain domain (VH) comprising the amino acid sequence of SEQ ID NO: 8 and a constant heavy chain domain 1 (CH1) (site-specific binding domain) and an Fc region comprising the amino acid sequence of SEQ ID NO: 12);
the amino acid sequence of SEQ ID NOs: 9 and 10 (“Chain 2” which comprises a variable light chain domain (VL) (site-specific binding domain) comprising the amino acid sequence of SEQ ID NO: 9 and a constant light chain domain (CL) comprising the amino acid sequence of SEQ ID NO: 10); and
the amino acid sequence of SEQ ID NO: 19 (“Chain 3” which comprises an Fc region).

In specific embodiments, an antigen-binding molecule of the present invention comprises:
the amino acid sequence of SEQ ID NOs: 8 and 13 (“chain 1” which comprises a variable heavy chain domain (VH) comprising the amino acid sequence of SEQ ID NO: 8 and a constant heavy chain domain 1 (CH1) (site-specific binding domain) and an Fc region comprising the amino acid sequence of SEQ ID NO: 13);
the amino acid sequence of SEQ ID NOs: 9 and 10 (“Chain 2” which comprises a variable
the amino acid sequence of SEQ ID NO: 20 (“Chain 3” which comprises an Fc region).

In specific embodiments, an antigen-binding molecule of the present invention comprises:
the amino acid sequence of SEQ ID NOs: 8 and 14 (“chain 1” which comprises a variable heavy chain domain (VH) comprising the amino acid sequence of SEQ ID NO: 8 and a constant heavy chain domain 1 (CH1) (site-specific binding domain) and an Fc region comprising the amino acid sequence of SEQ ID NO: 14);
the amino acid sequence of SEQ ID NOs: 9 and 10 (“Chain 2” which comprises a variable light chain domain (VL) (site-specific binding domain) comprising the amino acid sequence of SEQ ID NO: 9 and a constant light chain domain (CL) comprising the amino acid sequence of SEQ ID NO: 10); and
the amino acid sequence of SEQ ID NO: 21 (“Chain 3” which comprises an Fc region).

In specific embodiments, an antigen-binding molecule of the present invention comprises:
the amino acid sequence of SEQ ID NOs: 8 and 15 (“chain 1” which comprises a variable heavy chain domain (VH) comprising the amino acid sequence of SEQ ID NO: 8 and a constant heavy chain domain 1 (CH1) (site-specific binding domain) and an Fc region comprising the amino acid sequence of SEQ ID NO: 15);
the amino acid sequence of SEQ ID NOs: 9 and 10 (“Chain 2” which comprises a variable light chain domain (VL) (site-specific binding domain) comprising the amino acid sequence of SEQ ID NO: 9 and a constant light chain domain (CL) comprising the amino acid sequence of SEQ ID NO: 10); and
the amino acid sequence of SEQ ID NO: 22 (“Chain 3” which comprises an Fc region).

In specific embodiments, an antigen-binding molecule of the present invention comprises:
the amino acid sequence of SEQ ID NOs: 8 and 16 (“chain 1” which comprises a variable heavy chain domain (VH) comprising the amino acid sequence of SEQ ID NO: 8 and a constant heavy chain domain 1 (CH1) (site-specific binding domain) and an Fc region comprising the amino acid sequence of SEQ ID NO: 16);
the amino acid sequence of SEQ ID NOs: 9 and 10 (“Chain 2” which comprises a variable
the amino acid sequence of SEQ ID NO: 23 (“Chain 3” which comprises an Fc region).

The antigen-binding molecules of the present disclosure also include an antibody which has undergone posttranslational modification. Examples of the antigen-binding molecules of the present disclosure which have undergone posttranslational modification include antibodies which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

### Antigen

As used herein, the term “antigen” refers to the whole of or a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of viruses, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein can be any native form the proteins from any vertebrate source, including mammals such as primates (e.g. humans) and rodents (e.g. mice and rats), unless otherwise indicated. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

In one embodiment, the antigen of the present disclosure is a human antigen or cynomolgus antigen or mouse antigen, most particularly human antigen. The antigen may be ligands (cytokines, chemokines, and such), receptors, cancer antigens, MHC antigens, differentiation antigens, immunoglobulins, and immune complexes partly containing immunoglobulins. In specific embodiments, the antigen in HER2.

In one embodiment, the antigen of the present disclosure is a virus or a viral protein. In some embodiments, the virus of the present disclosure is preferably selected from an adenovirus, an astrovirus, a hepadnavirus, a herpesvirus, a papovavirus, a poxvirus, an arenavirus, a bunyavirus, a calcivirus, a coronavirus, a filovirus, a flavivirus, an orthomyxovirus, a paramyxovirus, a picornavirus, a reovirus, a retrovirus, a rhabdovirus, or a togavirus.

In preferred embodiments, the adenovirus includes, but is not limited to, a human adenovirus. In preferred embodiments, the astrovirus includes, but is not limited to, a mamastrovirus. In preferred embodiments, the hepadnavirus includes, but is not limited to, the hepatitis B virus. In preferred embodiments, the herpesvirus includes, but is not limited to, a herpes simplex virus type I, a herpes simplex virus type 2, a human cytomegalovirus, an Epstein-Barr virus, a varicella zoster virus, a roseolovirus, and a Kaposi's sarcoma-associated herpesvirus. In preferred embodiments, the papovavirus includes, but is not limited to, human papilloma virus and a human polyoma virus. In preferred embodiments, the poxvirus includes, but is not limited to, a variola virus, a vaccinia virus, a cowpox virus, a monkeypox virus, a smallpox virus, a pseudocowpox virus, a papular stomatitis virus, a tanapox virus, a yaba monkey tumor virus, and a molluscum contagiosum virus. In preferred embodiments, the arenavirus includes, but is not limited to lymphocytic choriomeningitis virus, a lassa virus, a machupo virus, and a junin virus. In preferred embodiments, the bunyavirus includes, but is not limited to, a hanta virus, a nairovirus, an orthobunyavirus, and a phlebovirus. In preferred embodiments, the calcivirus includes, but is not limited to, a vesivirus, a norovirus, such as the Norwalk virus and a sapovirus. In preferred embodiments, the coronavirus includes, but is not limited to, a human coronavirus (etiologic agent of severe acute respiratory syndrome (SARS)), and severe acute respiratory syndrome coronavirus 2 (SARS-CoV-2). In preferred embodiments, the filovirus includes, but is not limited to, an Ebola virus and a Marburg virus. In preferred embodiments, the flavivirus includes, but is not limited to, a yellow fever virus, a West Nile virus, a dengue virus (DENY-1, DENV-2, DENV-3 and DENV-4), a hepatitis C virus, a tick borne encephalitis virus, a Japanese encephalitis virus, a Murray Valley encephalitis virus, a St. Louis encephalitis virus, a Russian spring-summer encephalitis virus, a Omsk hemorrhagic fever virus, a bovine viral diarrhea virus, a Kyasanus Forest disease virus, and a Powassan encephalitis virus. In preferred embodiments, the orthomyxovirus includes, but is not limited to, influenza virus type A, influenza virus type B, and influenza virus type C. In preferred embodiments, the paramyxovirus includes, but is not limited to, a parainfluenza virus, a rubula virus (mumps), a morbillivirus (measles), a pneumovirus, such as a human respiratory syncytial virus, and a subacute sclerosing panencephalitis virus. In preferred embodiments, the picornavirus includes, but is not limited to, a poliovirus, a rhinovirus, a coxsackievirus A, a coxsackievirus B, a hepatitis A virus, an echovirus, and an enterovirus. In preferred embodiments, the reovirus includes, but is not limited to, a Colorado tick fever virus and a rotavirus. In preferred embodiments, the retrovirus includes, but is not limited to, a lentivirus, such as a human immunodeficiency virus, and a human T-lymphotrophic virus (HTLV). In preferred embodiments, the rhabdovirus includes, but is not limited to, a lyssavirus, such as the rabies virus, the vesicular stomatitis virus and the infectious hematopoietic necrosis virus. In preferred embodiments, the togavirus includes, but is not limited to, an alphavirus, such as a Ross river virus, an O'nyong'nyong virus, a Sindbis virus, a Venezuelan equine encephalitis virus, an Eastern equine encephalitis virus, and a Western equine encephalitis virus, and a rubella virus. In one non-limiting embodiment, the virus of the present disclosure is any virus which might cause the risk of Antibody-dependent enhancement (ADE).

### Antigen-Binding Domain (or Antigen-Binding Moiety)

In one embodiment, “antigen-binding domain” or “antigen-binding moiety” of the present disclosure refers to the part of an antibody that comprises the area which specifically binds to and is complementary to a part or all of an antigen. An antigen-binding domain may be provided by, for example, one or more antibody variable domains (also called antibody variable regions). Preferably, the antigen-binding domains contain both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F (ab′)2”. An antigen-binding domain may also be provided by single-domain antibodies.

### Single-Domain Antibody

In the present specification, the term “single-domain antibody” is not limited by its structure as long as the domain can exert antigen binding activity by itself. It is known that a general antibody, for example, an IgG antibody, exhibits antigen binding activity in a state where a variable region is formed by the pairing of VH and VL, whereas the own domain structure of the single-domain antibody can exert antigen binding activity by itself without pairing with another domain. Usually, the single-domain antibody has a relatively low molecular weight and exists in the form of a monomer.

Examples of the single-domain antibody include, but are not limited to, antigen-binding molecules congenitally lacking a light chain, such as VHH of an animal of the family Camelidae and shark VNAR, and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present invention, one single-domain antibody has three CDRs (CDR1, CDR2 and CDR3).

The single-domain antibody can be obtained from an animal capable of producing the single-domain antibody or by the immunization of the animal capable of producing the single-domain antibody. Examples of the animal capable of producing the single-domain antibody include, but are not limited to, animals of the family Camelidae, and transgenic animals harboring a gene capable of raising the single-domain antibody. The animals of the family Camelidae include camels, lamas, alpacas, one-hump camels and guanacos, etc. Examples of the transgenic animals harboring a gene capable of raising the single-domain antibody include, but are not limited to, transgenic animals described in International Publication No. WO2015/143414 and U.S. Patent Publication No. US2011/0123527 A1. The framework sequences of the single-domain antibody obtained from the animal may be converted to human germline sequences or sequences similar thereto to obtain a humanized single-domain antibody. The humanized single-domain antibody (e.g., humanized VHH) is also one embodiment of the single-domain antibody of the present invention.

Alternatively, the single-domain antibody can be obtained by ELISA, panning, or the like from a polypeptide library containing single-domain antibodies. Examples of the polypeptide library containing single-domain antibodies include, but are not limited to, naive antibody libraries obtained from various animals or humans (e.g., Methods in Molecular Biology 2012 911 (65-78); and Biochimica et Biophysica Acta—Proteins and Proteomics 2006 1764: 8 (1307-1319)), antibody libraries obtained by the immunization of various animals (e.g., Journal of Applied Microbiology 2014 117: 2 (528-536)), and synthetic antibody libraries prepared from antibody genes of various animals or humans (e.g., Journal of Biomolecular Screening 2016 21: 1 (35-43); Journal of Biological Chemistry 2016 291:24 (12641-12657); and AIDS 2016 30: 11 (1691-1701)).

### Variable Region

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### HVR or CDR

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Hypervariable regions (HVRs) are also referred to as “complementarity determining regions” (CDRs), and these terms are used herein interchangeably in reference to portions of the variable region that form the antigen binding regions. Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 are also mentioned as “H-CDR1”, “H-CDR2”, “H-CDR3”, “L-CDR1”, “L-CDR2”, and “L-CDR3”, respectively.

### Fab Molecule

A “Fab molecule” refers to a protein consisting of the VH and CH1 domain of the heavy chain (the “Fab heavy chain”) and the VL and CL domain of the light chain (the “Fab light chain”) of an immunoglobulin.

### Fused

By “fused” is meant that the components (e.g. a Fab molecule and an Fc domain subunit) are linked by peptide bond(s), either directly or via one or more peptide linkers.

### “Crossover” Fab

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

### “Conventional” Fab

In contrast thereto, by a “conventional” Fab molecule is meant a Fab molecule in its natural format, i.e. comprising a heavy chain composed of the heavy chain variable and constant regions (VH-CH1), and a light chain composed of the light chain variable and constant regions (VL-CL). The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CHL CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called alpha (IgA), delta (IgD), epsilon (IgE), gamma (IgG), or mu (IgM), some of which may be further divided into subtypes, e.g. gamma 1 (IgG1), gamma 2 (IgG2), gamma 3 (IgG3), gamma 4 (IgG4), alpha 1 (IgA1) and alpha 2 (IgA2). The light chain of an immunoglobulin may be assigned to one of two types, called kappa and lambda, based on the amino acid sequence of its constant domain. An immunoglobulin essentially consists of two Fab molecules and an Fc domain, linked via the immunoglobulin hinge region.

### Affinity/Avidity

“Affinity” refers to the strength of the sum total of noncovalent interactions between a single binding site of a molecule (e.g., an antigen-binding molecule or antibody) and its binding partner (e.g., an antigen). Unless indicated otherwise, as used herein, “binding affinity” refers to intrinsic binding affinity which reflects a 1:1 interaction between members of a binding pair (e.g., antigen-binding molecule and antigen, or antibody and antigen). The affinity of a molecule X for its partner Y can generally be represented by the dissociation constant (KD), which is the ratio of dissociation and association rate constants (k_{off} and kₒₙ, respectively). Thus, equivalent affinities may comprise different rate constants, as long as the ratio of the rate constants remains the same. Affinity can be measured by well-established methods known in the art, including those described herein. A particular method for measuring affinity is Surface Plasmon Resonance (SPR).

The structure of the antigen-binding domain of an antibody that binds to the epitope is called paratope. The paratope stably binds to the epitope through a hydrogen bond, electrostatic force, van der Waals' forces, a hydrophobic bond, or the like acting between the epitope and the paratope. This binding force between the epitope and the paratope is called “affinity” (see also above). The total binding force when a plurality of antigen binding domains bind to a plurality of antigens is called “avidity”. The affinity works synergistically when, for example, an antibody comprising a plurality of antigen binding domains (i.e., a polyvalent or a multivalent antibody) bind to a plurality of epitopes, and the avidity may be higher than the affinity.

### Methods to Determine Affinity

In certain embodiments, the antigen-binding molecule or antibody provided herein has a dissociation constant (KD) of 1 micromolar (micro M) or less, 120 nM or less, 100 nM or less, 80 nM or less, 70 nM or less, 50 nM or less, 40 nM or less, 30 nM or less, 20 nM or less, 10 nM or less, 2 nM or less, 1 nM or less, 0.1 nM or less, 0.01 nM or less, or 0.001 nM or less (e.g., 10⁻⁸M or less, 10⁻⁸M to 10⁻¹³M, 10⁻⁹M to 10⁻¹³ M) for its antigen. In certain embodiments, the KD value of the antibody/antigen-binding molecule for an antigen falls within the range of 1-40, 1-50, 1-70, 1-80, 30-50, 30-70, 30-80, 40-70, 40-80, or 60-80 nM.

In one embodiment, KD is measured by a radiolabeled antigen-binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 microgram (micro g)/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 microliters (micro 1)/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE(registered trademark)-3000 (BIAcore, Inc., Piscataway, N.J.) is performed at 25 degrees C. with immobilized antigen CMS chips at approximately 10 response units (RU). In one embodiment, carboxymethylated dextran biosensor chips (CMS, BIACORE, Inc.) are activated with N-ethyl-N-(3-dimethyl aminopropyl)-carbodiimide hydrochloride (EDC) and N-hydroxysuccinimide (NHS) according to the supplier's instructions. Antigen is diluted with 10 mM sodium acetate, pH 4.8, to 5 micro g/ml (approximately 0.2 micro M) before injection at a flow rate of 5 micro 1/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro l/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

According to the methods for measuring the affinity of the antigen-binding molecule or the antibody described above, persons skilled in art can carry out affinity measurement for other antigen-binding molecules or antibodies, towards various kind of antigens.

### Antibody

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

### Class of Antibody

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

Unless otherwise indicated, amino acid residues in the light chain constant region are numbered herein according to Kabat et al., and numbering of amino acid residues in the heavy chain constant region is according to the EU numbering system, also called the EU index, as described in Kabat et al., Sequences of Proteins of Immunological Interest, 5th Ed. Public Health Service, National Institutes of Health, Bethesda, Md., 1991.

### Framework

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

### Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

### Chimeric Antibody

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “humanized antibody variable region” refers to the variable region of a humanized antibody.

### Human Antibody

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. A “human antibody variable region” refers to the variable region of a human antibody.

### Polynucleotide (Nucleic Acid)

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂(“amidate”), P(O)R, P(O)OR, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

### Isolated (Nucleic Acid)

An “isolated” nucleic acid molecule is one which has been separated from a component of its natural environment. An isolated nucleic acid molecule further includes a nucleic acid molecule contained in cells that ordinarily contain the nucleic acid molecule, but the nucleic acid molecule is present extrachromosomally or at a chromosomal location that is different from its natural chromosomal location.

### Vector

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors could be introduced into host cells using virus or electroporation. However, introduction of vectors is not limited to in vitro method. For example, vectors could also be introduced into a subject using in vivo method directly.

### Host Cell

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

### Specificity

“Specific” means that a molecule that binds specifically to one or more binding partners does not show any significant binding to molecules other than the partners. Furthermore, “specific” is also used when an antigen-binding site is specific to a particular epitope of multiple epitopes contained in an antigen. If an antigen-binding molecule binds specifically to an antigen, it is also described as “the antigen-binding molecule has/shows specificity to/towards the antigen”. When an epitope bound by an antigen-binding site is contained in multiple different antigens, an antigen-binding molecule containing the antigen-binding site can bind to various antigens that have the epitope.

### Antibody Fragment

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

### Variable Fragment (Fv)

Herein, the term “variable fragment (Fv)” refers to the minimum unit of an antibody-derived antigen-binding site that is composed of a pair of the antibody light chain variable region (VL) and antibody heavy chain variable region (VH). In 1988, Skerra and Pluckthun found that homogeneous and active antibodies can be prepared from the E. coli periplasm fraction by inserting an antibody gene downstream of a bacterial signal sequence and inducing expression of the gene in E. coli (Science (1988) 240(4855), 1038-1041). In the Fv prepared from the periplasm fraction, VH associates with VL in a manner so as to bind to an antigen.

scFv, Single-Chain Antibody, and Sc(Fv)

Herein, the terms “scFv”, “single-chain antibody”, and “sc(Fv)₂” all refer to an antibody fragment of a single polypeptide chain that contains variable regions derived from the heavy and light chains, but not the constant region. In general, a single-chain antibody also contains a polypeptide linker between the VH and VL domains, which enables formation of a desired structure that is thought to allow antigen-binding. The single-chain antibody is discussed in detail by Pluckthun in “The Pharmacology of Monoclonal Antibodies, Vol. 113, Rosenburg and Moore, eds., Springer-Verlag, New York, 269-315 (1994)”. See also International Patent Publication WO 1988/001649; U.S. Pat. Nos. 4,946,778 and 5,260,203. In a particular embodiment, the single-chain antibody can be bispecific and/or humanized.

An scFv is a single chain low molecule weight antibody in which VH and VL forming Fv are linked together by a peptide linker (Proc. Natl. Acad. Sci. U.S.A. (1988) 85(16), 5879-5883). VH and VL can be retained in close proximity by the peptide linker. sc(Fv)₂ is a single chain antibody in which four variable regions of two VL and two VH are linked by linkers such as peptide linkers to form a single chain (J Immunol. Methods (1999) 231(1-2), 177-189). The two VH and two VL may be derived from different monoclonal antibodies. Such sc(Fv)₂ preferably includes, for example, a bispecific sc(Fv)₂ that recognizes two epitopes present in a single antigen as disclosed in the Journal of Immunology (1994) 152(11), 5368-5374. sc(Fv)₂ can be produced by methods known to those skilled in the art. For example, sc(Fv)₂ can be produced by linking scFv by a linker such as a peptide linker.

Herein, an sc(Fv)2 includes two VH units and two VL units which are arranged in the order of VH, VL, VH, and VL ([VH]-linker-[VL]-linker-[VH]-linker-[VL]) beginning from the N terminus of a single-chain polypeptide. The order of the two VH units and two VL units is not limited to the above form, and they may be arranged in any order. Examples of the form are listed below.
[VL]-linker-[VH]-linker-[VH]-linker-[VL]
[VH]-linker-[VL]-linker-[VL]-linker-[VH]
[VH]-linker-[VH]-linker-[VL]-linker-[VL]
[VL]-linker-[VL]-linker-[VH]-linker-[VH]
[VL]-linker-[VH]-linker-[VL]-linker-[VH]

The molecular form of sc(Fv)2 is also described in detail in WO 2006/132352. According to these descriptions, those skilled in the art can appropriately prepare desired sc(Fv)₂.

Furthermore, the antigen-binding molecules or antibodies of the present disclosure may be conjugated with a carrier polymer such as PEG or an organic compound such as an anticancer agent. Alternatively, a sugar chain addition sequence is preferably inserted into the antigen-binding molecules or antibodies such that the sugar chain produces a desired effect.

The linkers to be used for linking the variable regions of an antibody comprise arbitrary peptide linkers that can be introduced by genetic engineering, synthetic linkers, and linkers disclosed in, for example, Protein Engineering, 9(3), 299-305, 1996. However, peptide linkers are preferred in the present disclosure. The length of the peptide linkers is not particularly limited, and can be suitably selected by those skilled in the art according to the purpose. The length is preferably five amino acids or more (without particular limitation, the upper limit is generally 30 amino acids or less, preferably 20 amino acids or less), and particularly preferably 15 amino acids. When sc(Fv)₂ contains three peptide linkers, their length may be all the same or different.

For example, such peptide linkers include:

Ser,
Gly-Ser,
Gly-Gly-Ser,
Ser-Gly-Gly,
(SEQ ID NO: 26)
Gly-Gly-Gly-Ser,
(SEQ ID NO: 27)
Ser-Gly-Gly-Gly,
(SEQ ID NO: 28)
Gly-Gly-Gly-Gly-Ser,
(SEQ ID NO: 29)
Ser-Gly-Gly-Gly-Gly,
(SEQ ID NO: 30)
Gly-Gly-Gly-Gly-Gly-Ser,
(SEQ ID NO: 31)
Ser-Gly-Gly-Gly-Gly-Gly,
(SEQ ID NO: 32)
Gly-Gly-Gly-Gly-Gly-Gly-Ser,
(SEQ ID NO: 33)
Ser-Gly-Gly-Gly-Gly-Gly-Gly,
(Gly-Gly-Gly-Gly-Ser (SEQ ID NO: 28))n,
and
(Ser-Gly-Gly-Gly-Gly (SEQ ID NO: 29))n,

where n is an integer of 1 or larger. The length or sequences of peptide linkers can be selected accordingly by those skilled in the art depending on the purpose.

Synthetic linkers (chemical crosslinking agents) are routinely used to crosslink peptides, and examples include:
N-hydroxy succinimide (NHS),
disuccinimidyl suberate (DSS),
bis(sulfosuccinimidyl) suberate (BS3),
dithiobis(succinimidyl propionate) (DSP),
dithiobis(sulfosuccinimidyl propionate) (DTS SP),
ethylene glycol bis(succinimidyl succinate) (EGS),
ethylene glycol bis(sulfosuccinimidyl succinate) (sulfo-EGS),
disuccinimidyl tartrate (DST), disulfosuccinimidyl tartrate (sulfo-DST),
bis[2-(succinimidoxycarbonyloxy)ethyl] sulfone (BSOCOES), and
bis[2-(sulfosuccinimidoxycarbonyloxy)ethyl] sulfone (sulfo-BSOCOES). These crosslinking agents are commercially available.

In general, three linkers are required to link four antibody variable regions together. The linkers to be used may be of the same type or different types.

### Fab, F(ab′)₂, and Fab′

“Fab” consists of a single light chain, and a CH1 domain and variable region from a single heavy chain. The heavy chain of Fab molecule cannot form disulfide bonds with another heavy chain molecule.

“F(ab′)2” or “Fab” is produced by treating an immunoglobulin (monoclonal antibody) with a protease such as pepsin and papain, and refers to an antibody fragment generated by digesting an immunoglobulin (monoclonal antibody) near the disulfide bonds present between the hinge regions in each of the two H chains. For example, papain cleaves IgG upstream of the disulfide bonds present between the hinge regions in each of the two H chains to generate two homologous antibody fragments, in which an L chain comprising VL (L-chain variable region) and CL (L-chain constant region) is linked to an H-chain fragment comprising VH (H-chain variable region) and CH gamma 1 (gamma 1 region in an H-chain constant region) via a disulfide bond at their C-terminal regions. Each of these two homologous antibody fragments is called Fab′.

“F(ab′)₂” consists of two light chains and two heavy chains comprising the constant region of a CH1 domain and a portion of CH2 domains so that disulfide bonds are formed between the two heavy chains. The F(ab′)₂ disclosed herein can be preferably produced as follows. A whole monoclonal antibody or such comprising a desired antigen-binding site is partially digested with a protease such as pepsin; and Fc fragments are removed by adsorption onto a Protein A column. The protease is not particularly limited, as long as it can cleave the whole antibody in a selective manner to produce F(ab′)₂ under an appropriate setup enzyme reaction condition such as pH. Such proteases include, for example, pepsin and ficin.

### C1q

“C1q” is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e. g. from Quidel, San Diego, Calif.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with an Fc region variant) and increased or decreased C1q binding capability are described, e.g., in U.S. Pat. No. 6,194,551 B1 and WO 1999/51642. See also, e.g., Idusogie et al. J. Immunol. 164: 4178-4184 (2000). “Complement dependent cytotoxicity” or “CDC” herein may further refer to the lysis of a target virus (virolysis) or reduction of the virus' ability to infect cells by complement. Methods for assessing complement dependent lysis or complement dependent reduction of virus infectivity are widely known in the art, such as the use of heat inactivated serum or serum depleted of complement components. Examples of complement dependent virolysis or inactivation are detailed in Springer Semin Immunopathol. 1983; 6(4): 327-347.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The phrase “substantially decreased”, “substantially increased”, or “substantially different”, as used herein, refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values).

### Fc Polypeptide

In the present specification, the term “Fc polypeptide” is not limited by its structure as long as the Fc polypeptide comprises a C-terminal region of an immunoglobulin heavy chain that contains at least a portion of the constant region, CH2 domain, CH3 domain, CH2 and CH3 domains, Fc region, or variant thereof.

The term “Fc region” or “Fc domain” herein is used to define a C-terminal region of an immunoglobulin heavy chain that contains at least a portion of the constant region. The term includes native sequence Fc regions and variant Fc regions. In one embodiment, the term “Fc region” or “Fc domain” comprises a fragment consisting of a hinge or a portion thereof and CH2 and CH3 domains in an antibody molecule. The Fc region of IgG class means, but is not limited to, a region from, for example, cysteine 226 (EU numbering (also referred to as EU index herein)) to the C terminus or proline 230 (EU numbering) to the C terminus. The Fc region can be preferably obtained by the partial digestion of, for example, an IgG1, IgG2, IgG3, or IgG4 monoclonal antibody with a proteolytic enzyme such as pepsin followed by the re-elution of a fraction adsorbed on a protein A column or a protein G column. Such a proteolytic enzyme is not particularly limited as long as the enzyme is capable of digesting a whole antibody to restrictively form Fab or F(ab′)2 under appropriately set reaction conditions (e.g., pH) of the enzyme. Examples thereof can include pepsin and papain.

An Fc region derived from, for example, naturally occurring (wild type) IgG can be used as the “Fc region” of an antigen-binding molecule. In this context, the naturally occurring IgG means a polypeptide that contains an amino acid sequence identical to that of IgG found in nature and belongs to a class of an antibody substantially encoded by an immunoglobulin gamma gene. The naturally occurring human IgG means, for example, naturally occurring human IgG1, naturally occurring human IgG2, naturally occurring human IgG3, or naturally occurring human IgG4. The naturally occurring IgG also includes variants or the like spontaneously derived therefrom. A plurality of allotype sequences based on gene polymorphism are described as the constant regions of human IgG1, human IgG2, human IgG3, and human IgG4 antibodies in Sequences of proteins of immunological interest, NIH Publication No. 91-3242, any of which can be used in the present disclosure. Particularly, the sequence of human IgG1 may have DEL or EEM as an amino acid sequence of EU numbering positions 356 to 358.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain (see, e.g., Daeron, Annu. Rev. Immunol. 15:203-234 (1997)). FcRs are reviewed, for example, in Ravetch and Kinet, Annu. Rev Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half-life of human FcRn high affinity binding polypeptides can be assayed, e.g., in transgenic mice or transfected human cell lines expressing human FcRn, or in primates to which the polypeptides with an Fc region variant are administered. WO 2000/42072 (Presta) describes antibody variants with increased or decreased binding to FcRs. See also, e.g., Shields et al. J. Biol. Chem. 9(2):6591-6604 (2001).

### Fc Gamma Receptor

Fc gamma receptor refers to a receptor capable of binding to the Fc domain of monoclonal IgG1, IgG2, IgG3, or IgG4 antibodies, and includes all members belonging to the family of proteins substantially encoded by an Fc gamma receptor gene. In human, the family includes Fc gamma RI (CD64) including isoforms Fc gamma RIa, Fc gamma RIb and Fc gamma RIc; Fc gamma RII (CD32) including isoforms Fc gamma RIIa (including allotype H131 and R131), Fc gamma RIIb (including Fc gamma RIIb-1 and Fc gamma RIIb-2), and Fc gamma RIIc; and Fc gamma RIII (CD16) including isoform Fc gamma RIIIa (including allotype V158 and F158) and Fc gamma RIIIb (including allotype Fc gamma RIIIb-NA1 and Fc gamma RIIIb-NA2); as well as all unidentified human Fc gamma receptors, Fc gamma receptor isoforms, and allotypes thereof. However, Fc gamma receptor is not limited to these examples. Without being limited thereto, Fc gamma receptor includes those derived from humans, mice, rats, rabbits, and monkeys. Fc gamma receptor may be derived from any organisms. Mouse Fc gamma receptor includes, without being limited to, Fc gamma RI (CD64), Fc gamma RII (CD32), Fc gamma RIII (CD16), and Fc gamma RIII-2 (CD16-2), as well as all unidentified mouse Fc gamma receptors, Fc gamma receptor isoforms, and allotypes thereof. Such preferred Fc gamma receptors include, for example, human Fc gamma RI (CD64), Fc gamma RIIA (CD32), Fc gamma RIIB (CD32), Fc gamma RIIIA (CD16), and/or Fc gamma RIIIB (CD16). The polynucleotide sequence and amino acid sequence of Fc gamma RI are shown in RefSeq accession number NM_000566.3 and RefSeq accession number NP_000557.1, respectively; the polynucleotide sequence and amino acid sequence of Fc gamma RIIA are shown in RefSeq accession number BCO20823.1 and RefSeq accession number AAH20823.1, respectively; the polynucleotide sequence and amino acid sequence of Fc gamma RIIB are shown in RefSeq accession number BC146678.1 and RefSeq accession number AAI46679.1, respectively; the polynucleotide sequence and amino acid sequence of Fc gamma RIIIA are shown in RefSeq accession number BC033678.1 and RefSeq accession number AAH33678.1, respectively; and the polynucleotide sequence and amino acid sequence of Fc gamma RIIIB are shown in RefSeq accession number BC128562.1 and RefSeq accession number AAI28563.1, respectively. Whether an Fc gamma receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103(11), 4005-4010), in addition to the above-described FACS and ELISA formats.

Meanwhile, “Fc ligand” or “effector ligand” refers to a molecule and preferably a polypeptide that binds to an antibody Fc domain, forming an Fc/Fc ligand complex. The molecule may be derived from any organisms. The binding of an Fc ligand to Fc preferably induces one or more effector functions. Such Fc ligands include, but are not limited to, Fc receptors, Fc gamma receptor, Fc alpha receptor, Fc beta receptor, FcRn, C1q, and C3, mannan-binding lectin, mannose receptor, Staphylococcus Protein A, Staphylococcus Protein G, and viral Fc gamma receptors. The Fc ligands also include Fc receptor homologs (FcRH) (Davis et al., (2002) Immunological Reviews 190, 123-136), which are a family of Fc receptors homologous to Fc gamma receptor. The Fc ligands also include unidentified molecules that bind to Fc.

### Fc Gamma Receptor-Binding Activity

The impaired binding activity of Fc domain to any of the Fc gamma receptors Fc gamma RI, Fc gamma RIIA, Fc gamma RIIB, Fc gamma RIIIA, and/or Fc gamma RIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103(11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fc gamma receptor is immobilized to the acceptor beads. In the absence of an antigen-binding molecule or antibody comprising a competitive mutant Fc domain, Fc gamma receptor interacts with an antigen-binding molecule or antibody comprising a wild-type Fc domain, inducing a signal of 520 to 620 nm as a result. The antigen-binding molecule or antibody having a non-tagged mutant Fc domain competes with the antigen-binding molecule or antibody comprising a wild-type Fc domain for the interaction with Fc gamma receptor. The relative binding affinity can be determined by quantifying the reduction of fluorescence as a result of competition. Methods for biotinylating the antigen-binding molecules or antibodies such as antibodies using Sulfo-NHS-biotin or the like are known. Appropriate methods for adding the GST tag to an Fc gamma receptor include methods that involve fusing polypeptides encoding Fc gamma receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE® methods. Examples of such inhibition assay are described in Proc. Natl. Acad. Sci. USA (2006) 103(11), 4005-4010.

### Fc Region Variants (or Variant Fc Regions/Fc Domain Variants/Variant Fc Domains)

In one aspect, the Fc polypeptide of the present disclosure comprises a first Fc region variant and a second Fc region variant, each comprising at least one amino acid alteration (or mutation or modification, including substitution) relative to a parent Fc region. Such Fc polypeptide comprising the first Fc region variant and the second Fc region variant may be called “variant Fc polypeptide” in this disclosure.

In certain embodiments, one or more amino acid alterations (mutations or modifications, including amino acid substitutions, deletions, and insertions) may be introduced into an Fc region of an antibody (a parent Fc region), thereby generating an Fc region variant. The Fc region variant may comprise a human Fc region sequence (e.g., a human IgG1, IgG2, IgG3, or IgG4 Fc region) comprising an amino acid modification (e.g. a substitution) at one or more amino acid positions. For instance, the heavy chain constant region of human IgG1, human IgG2, human IgG3, and human IgG4 are shown in SEQ ID NOs: 63 to 66, respectively. For instance, the Fc region of human IgG1, human IgG2, human IgG3, and human IgG4 are shown as a partial sequence of SEQ ID NOs: 63 to 66.

In certain embodiments, the present disclosure contemplates an antigen-binding molecule that possesses some but not all effector functions, which make it a desirable candidate for applications in which the half-life of the antibody in vivo is important yet certain effector functions (such as ADCC) are unnecessary or deleterious. In vitro and/or in vivo cytotoxicity assays can be conducted to measure CDC and/or ADCC activities. For example, Fc receptor (FcR) binding assays can be conducted to confirm whether the antibody has Fc gamma R binding (hence likely having ADCC activity) and/or FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assay methods may be employed (see, for example, ACT1 ™ non-radioactive cytotoxicity assay for flow cytometry (CellTechnology, Inc. Mountain View, Calif.); and CytoTox 96 (registered trademark) non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95: 652-656 (1998). C1q binding assays may also be carried out to confirm whether the antibody is able to bind C1q and hence has CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). Known methods for assessing complement dependent lysis or complement dependent reduction of virus infectivity, such as the use of heat inactivated serum or serum depleted of complement components, may also be used to assess C1q binding/complement activation. FcRn binding and in vivo clearance/half-life determinations can also be performed using methods known in the art (see, e.g., Petkova, S. B. et al., Int'l. Immunol. 18(12):1759-1769 (2006)).

In certain embodiments, the first and/or second Fc region variants (collectively referred to herein as a “Fc region variant (s)” or “variant Fc region (s)”) comprise at least one amino acid residue alteration (e.g., substitution) compared to the corresponding sequence in the Fc region of a native or reference variant sequence (sometimes collectively referred to herein as a “parent” Fc region).

A “parent Fc region” as used herein refers to an Fc region prior to the introduction of an amino acid alteration(s) described herein. Preferred examples of the parent Fc region include Fc regions derived from native antibodies. Antibodies include, for example, IgA (IgA1, IgA2), IgD, IgE, IgG (IgG1, IgG2, IgG3, IgG4), and IgM, or such. Antibodies may be derived from human or monkey (e.g., cynomolgus, rhesus macaque, marmoset, chimpanzee, or baboon). Native antibodies may also include naturally-occurring mutations. A plurality of allotype sequences of IgGs due to genetic polymorphism are described in “Sequences of proteins of immunological interest”, NIH Publication No. 91-3242, and any of them may be used in the present invention. In particular, for human IgG1, the amino acid sequence at positions 356 to 358 (EU numbering) may be either DEL or EEM. Preferred examples of the parent Fc region include Fc regions derived from a heavy chain constant region of human IgG1 (SEQ ID NO: 63), human IgG2 (SEQ ID NO: 64), human IgG3 (SEQ ID NO: 65), and human IgG4 (SEQ ID NO: 66). Another preferred example of the parent Fc region is an Fc region derived from a heavy chain constant region SG1 (SEQ ID NO: 67). Another preferred example of the parent Fc region is an Fc region derived from a heavy chain constant region SG182 (SEQ ID NO: 48). Furthermore, the parent Fc region may be an Fc region produced by adding an amino acid alteration(s) other than the amino acid alteration(s) described herein to an Fc region derived from a native antibody.

In certain embodiments, the variant Fc region of the present disclosure has a substantially decreased Fc gamma receptor-binding activity compared to the parent Fc region. In certain embodiments, the variant Fc region of the present disclosure has a maintained (does not have a substantially decreased) C1q-binding activity or increased C1q-binding activity compared to the parent Fc region. In certain embodiments, Fc gamma receptor is human Fc gamma receptor, monkey Fc gamma receptor (e.g., cynomolgus, rhesus macaque, marmoset, chimpanzee, or baboon Fc gamma receptor), or mouse Fc gamma receptor.

In a variant Fc polypeptide (or an antigen-binding molecule comprising the variant Fc polypeptide) of the present disclosure having a substantially decreased binding activity for one or more human Fc gamma receptors, typically, the same one or more amino acid mutation is present in each of the two variant Fc regions constituting the Fc polypeptide of the antigen-binding molecule. In certain embodiments, the variant Fc region described herein exhibits reduced binding affinity to a Fc gamma receptor, as compared to a native IgG1 Fc region. Herein, human Fc gamma receptors (Fc gamma Rs) include, but are not limited to Fc gamma RIa, Fc gamma RIIa (including allelic variants 167H and 167R), Fc gamma RIIb, Fc gamma RIIIa (including allelic variants 158F and 158V), and Fc gamma RIIIb (including allelic variants NA1 and NA2). In a further aspect, a variant Fc region of the present disclosure has a substantially decreased binding activity for human Fc gamma RIa, Fc gamma RIIa (including allelic variants 167H and 167R), Fc gamma RIIb, Fc gamma RIIIa (including allelic variants 158F and 158V), and Fc gamma RIIIb (including allelic variants NA1 and NA2), as compared to a parent Fc region.

In one aspect, a variant Fc region of the present disclosure has a substantially decreased binding activity for one or more mouse Fc gamma Rs including, but not limited to Fc gamma RI, Fc gamma RIIb, Fc gamma RIII, and Fc gamma RIV, as compared to a parent Fc region. In a further aspect, a variant Fc region of the present disclosure has a substantially decreased binding activity for mouse Fc gamma RI, Fc gamma RIIb, Fc gamma RIII, and Fc gamma RIV, as compared to a parent Fc region.

“Fc gamma receptors” (herein, referred to as Fc gamma receptors, Fc gamma R or FcgR) refers to receptors that may bind to the Fc region of IgG1, IgG2, IgG3, and IgG4 monoclonal antibodies, and practically means any member of the family of proteins encoded by the Fc gamma receptor genes. In humans, this family includes Fc gamma RI (CD64) including isoforms Fc gamma RIa, Fc gamma RIb, and Fc gamma RIc; Fc gamma RII (CD32) including isoforms Fc gamma RIIa (including allotypes H131 (type H) and R131 (type R)), Fc gamma RIIb (including Fc gamma RIIb-1 and Fc gamma RIIb-2), and Fc gamma RIIc; and Fc gamma RIII (CD16) including isoforms Fc gamma RIIIa (including allotypes V158 and F158), and Fc gamma RIIIb (including allotypes Fc gamma RIIIb-NA1 and Fc gamma RIIIb-NA2), and any human Fc gamma Rs, Fc gamma R isoforms or allotypes yet to be discovered, but is not limited thereto. Fc gamma RIIb1 and Fc gamma RIIb2 have been reported as splicing variants of human Fc gamma RIIb. In addition, a splicing variant named Fc gamma RIIb3 has been reported (J Exp Med, 1989, 170: 1369-1385). In addition to these splicing variants, human Fc gamma RIIb includes all splicing variants registered in NCBI, which are NP_001002273.1, NP_001002274.1, NP_001002275.1, NP_001177757.1, and NP_003992.3. Furthermore, human Fc gamma RIIb includes every previously-reported genetic polymorphism, as well as Fc gamma RIIb (Arthritis Rheum. 48:3242-3252 (2003); Kono et al., Hum. Mol. Genet. 14:2881-2892 (2005); and Kyogoju et al., Arthritis Rheum. 46:1242-1254 (2002)), and every genetic polymorphism that will be reported in the future.

In Fc gamma RIIa, there are two allotypes, one where the amino acid at position 167 of Fc gamma RIIa is histidine (type H) and the other where the amino acid at position 167 is substituted with arginine (type R) (Warrmerdam, J. Exp. Med. 172:19-25 (1990)).

The Fc gamma R includes human, mouse, rat, rabbit, and monkey-derived Fc gamma Rs but is not limited thereto, and may be derived from any organism. Mouse Fc gamma Rs include Fc gamma RI (CD64), Fc gamma RII (CD32), Fc gamma RIII (CD16), and Fc gamma RIV (CD16-2), and any mouse Fc gamma Rs, or Fc gamma R isoforms, but are not limited thereto.

The amino acid sequence of human Fc gamma RIa is set forth in SEQ ID NO: 34; the amino acid sequence of human Fc gamma RIIa (167H) is set forth in SEQ ID NO: 35; the amino acid sequence of human Fc gamma RIIa (167R) is set forth in SEQ ID NO: 36; the amino acid sequence of human Fc gamma RIIb is set forth in SEQ ID NO: 37; the amino acid sequence of human Fc gamma RIIIa (158F) is set forth in SEQ ID NO: 38; the amino acid sequence of human Fc gamma RIIIa (158V) is set forth in SEQ ID NO: 39; the amino acid sequence of human Fc gamma RIIIb (NA1) is set forth in SEQ ID NO: 40; and the amino acid sequence of human Fc gamma RIIIb (NA2) is set forth in SEQ ID NO: 41.

The amino acid sequence of mouse Fc gamma RI is set forth in SEQ ID NO: 42; the amino acid sequence of mouse Fc gamma RIM is set forth in SEQ ID NO: 43; the amino acid sequence of mouse Fc gamma RIII is set forth in SEQ ID NO: 44; and the amino acid sequence of mouse Fc gamma RIV is set forth in SEQ ID NO: 45.

In one aspect, a variant Fc region of the present disclosure (or an antigen-binding molecule comprising said variant Fc region) has a substantially decreased Fc gamma R-binding activity that is less than 50%, less than 45%, less than 40%, less than 35%, less than 30%, less than 25%, less than 20%, less than 15%, less than 10%, less than 5%, less than 2%, less than 1%, less than 0.5%, less than 0.2%, or less than 0.1% as a function of the Fc gamma R-binding activity for the parent Fc region (or an antigen-binding molecule comprising said parent Fc region). In one aspect, a variant Fc region of the present disclosure has a substantially decreased Fc gamma R-binding activity, which means that the ratio of [the difference in the RU values of sensorgrams that changed before and after interaction of Fc gamma R with the variant Fc region]/[the difference in the RU values of sensorgrams that changed before and after capturing Fc gamma R to the sensor chips] is less than 1, less than 0.8, less than 0.5, less than 0.3, less than 0.2, less than 0.1, less than 0.08, less than 0.05, less than 0.03, less than 0.02, less than 0.01, less than 0.008, less than 0.005, less than 0.003, less than 0.002, or less than 0.001. In one embodiment, the variant Fc region (or the antigen-binding molecule comprising said variant Fc region) does not substantially bind to an Fc gamma receptor.

In one aspect, a variant Fc region of the present disclosure has a maintained (does not have a substantially decreased) C1q-binding activity or increased C1q-binding activity. “Maintained” or “not substantially decreased” C1q-binding activity means that the difference of C1q-binding activities between a variant Fc region and a parent Fc region of the present disclosure is less than 50%, less than 45%, less than 40%, less than 35%, less than 30%, less than 25%, less than 20%, less than 15%, less than 10%, or less than 5% as a function of the C1q-binding activity for the parent Fc region. In the case that a variant Fc region has an “increased” C1q-binding activity, the difference of C1q-binding activities between a variant Fc region and a parent Fc region of the present disclosure may be more than 50% and the variant Fc region of the present disclosure may have a C1q-binding activity that is 100% or more, 150% or more, 200% or more, 400% or more, 800% or more, or 1600% or more as a function of the C1q-binding activity for the parent Fc region. The comparison may be made at any concentration of antigen-binding molecule, but it is preferred that the comparison is made in the presence of a high concentration of antigen-binding molecule, which allows the antigen-binding molecule comprising a variant Fc region or a parent Fc region (control) to assemble into hexamers. Binding activity of an antigen-binding molecule to C1q can be evaluated using conventional C1q binding assay (e.g., C1q and C3c binding ELISA in WO 2006/029879 and WO 2005/100402) or by using a CDC assay (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). Known methods for assessing complement dependent lysis or complement dependent reduction of virus infectivity, such as the use of heat inactivated serum or serum depleted of complement components, may also be used to assess C1q binding.

Binding between hexameric antigen-binding molecules and C1q can be evaluated using known methods, such as ELISA-based methods, surface plasmon resonance (SPR)-based methods, etc. (see, e.g., Biologicals 2019 September; 61:76-79). Such assay may be carried out particularly under conditions that allow an antigen-binding molecule comprising a variant Fc region or a parent Fc region (control) to assemble into hexamers.

For example, to determine the binding activity of a polypeptide containing a variant Fc region towards C1q, a C1q binding ELISA may be performed. Briefly, assay plates may be coated overnight at 4 degrees C. with a polypeptide containing a variant Fc region or a polypeptide containing a parent Fc region (control) in coating buffer. The plates may then be washed and blocked. Following washing, an aliquot of human C1q may be added to each well and incubated for 2 hours at room temperature. Following a further wash, 100 microliters of a sheep anti-complement C1q peroxidase conjugated antibody may be added to each well and incubated for 1 hour at room temperature. The plate may again be washed with wash buffer and 100 microliters of substrate buffer containing OPD (o-phenylenediamine dihydrochloride (Sigma)) may be added to each well. The oxidation reaction, observed by the appearance of a yellow color, may be allowed to proceed for 30 minutes and stopped by the addition of 100 microliters of 4.5 N H₂SO₄. The absorbance may then read at (492-405) nm. The binding activity of an Fc region for C1q can be determined by a method described in WO2018/052375.

For another example, binding activity of an antigen-binding molecule to C1q can be evaluated using a CDC assay, as the occurrence of target lysis by CDC indicates the occurrence of the binding of C1q to an antibody Fc which triggers the classical complement pathway. CDC assay described in Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004) may suitably be used. For example, C1q binding may be assayed as detailed in the Examples of the present disclosure. Briefly, cells stably transfected to overexpress an antigen are suspended at a suitable concentration and seeded onto an assay plate. A suitable concentration of human serum is added to each well. Antibodies are diluted over a suitable range and added to each well. After mixing the components well, the plate is placed in an incubator and incubated at 37 degrees C. with 5% CO₂ for about 1 hour. The cells are washed with buffer and stained with a viability dye for example 7AAD, and analyzed by flow cytometry to determine the percentage of cells lysed by antibody-mediated CDC.

In one aspect, the present disclosure provides an antigen-binding molecule comprising a variant Fc region with a substantially decreased ADCC activity. In one aspect, the present disclosure provides an antigen-binding molecule comprising a variant Fc region with a maintained (without a substantially decreased) CDC activity or increased CDC activity. In one aspect, the present disclosure provides an antigen-binding molecule comprising a variant Fc region with a substantially decreased ADCC activity and a maintained (without a substantially decreased) CDC activity or increased CDC activity.

In one aspect, a variant Fc region of the present disclosure confers to an antigen-binding molecule comprising the variant Fc region a substantially decreased ADCC activity that is less than 50%, less than 45%, less than 40%, less than 35%, less than 30%, less than 25%, less than 20%, less than 15%, less than 10%, less than 5%, less than 2%, less than 1%, less than 0.5%, less than 0.2%, or less than 0.1% as a function of the ADCC activity for the antigen-binding molecule comprising the parent Fc region.

In one aspect, a variant Fc region of the present disclosure confers to an antigen-binding molecule comprising the variant Fc region a maintained (i.e., not substantially decreased) CDC activity or an increased CDC activity. “Maintained” or “not substantially decreased” CDC activity means that the difference of CDC activities between the antigen-binding molecule comprising the variant Fc region and the antigen-binding molecule comprising the parent Fc region is less than 50%, less than 45%, less than 40%, less than 35%, less than 30%, less than 25%, less than 20%, less than 15%, less than 10%, or less than 5%. In one aspect, a variant Fc region of the present disclosure confers to an antigen-binding molecule comprising the variant Fc region increased CDC activity that is more than 100%, more than 200%, more than 400%, more than 800%, or more than 1600% for the antigen-binding molecule comprising the parent Fc region, in which the CDC activity is determined as the concentration of the antibody required to achieve 50% of the maximum complement-dependent lysis of a target cell.

In further aspects, the variant Fc region of the present disclosure comprises at least one amino acid alteration of at least one position selected from the group consisting of: 234, 235, 236, 267, 268, 324, 326, 332, and 333, according to EU numbering (see, e.g., WO2018/052375).

In one aspect, the variant Fc region with a substantially decreased Fc gamma receptor-binding activity and a maintained (without a substantially decreased) or increased C1q-binding activity comprises Ala at position 234, Ala at position 235 and at least one amino acid alteration of at least one position selected from the group consisting of: 236, 267, 268, 324, 326, 332, and 333, according to EU numbering.

In one aspect, the variant Fc region with a substantially decreased Fc gamma receptor-binding activity and a maintained (without a substantially decreased) or increased C1q-binding activity comprises Ala at position 234, Ala at position 235 and further amino acid alterations of any one of the following (a)-(c): (a) positions 267, 268, and 324; (b) positions 236, 267, 268, 324, and 332; and (c) positions 326 and 333, according to EU numbering.

In a further aspect, the variant Fc region with a substantially decreased Fc gamma receptor-binding activity and maintained (without a substantially decreased) or increased C1q-binding activity comprises amino acids selected from the group consisting of: (a) Glu at position 267; (b) Phe at position 268; (c) Thr at position 324; (d) Ala at position 236; (e) Glu at position 332; (f) Ala, Asp, Glu, Met, or Trp at position 326; and (g) Ser at position 333, according to EU numbering.

In one aspect, the variant Fc region with a substantially decreased Fc gamma receptor-binding activity and maintained (without a substantially decreased) or increased C1q-binding activity comprises amino acids of: Ala at position 234, Ala at position 235, Ala at position 326, and Ser at position 333, according to EU numbering.

In one aspect, the variant Fc region with a substantially decreased Fc gamma receptor-binding activity and maintained (without a substantially decreased) or increased C1q-binding activity comprises amino acids of: Ala at position 234, Ala at position 235, Asp at position 326, and Ser at position 333, according to EU numbering.

In one aspect, the variant Fc region with a substantially decreased Fc gamma receptor-binding activity and maintained (without a substantially decreased) or increased C1q-binding activity comprises amino acids of: Ala at position 234, Ala at position 235, Glu at position 326, and Ser at position 333, according to EU numbering.

In one aspect, the variant Fc region with a substantially decreased Fc gamma receptor-binding activity and maintained (without a substantially decreased) or increased C1q-binding activity comprises amino acids of: Ala at position 234, Ala at position 235, Met at position 326, and Ser at position 333, according to EU numbering.

In one aspect, the variant Fc region with a substantially decreased Fc gamma receptor-binding activity and maintained (without a substantially decreased) or increased C1q-binding activity comprises amino acids of: Ala at position 234, Ala at position 235, Trp at position 326, and Ser at position 333, according to EU numbering.

In one aspect, the variant Fc region of the present disclosure has an increased FcRn binding activity under acidic pH, compared to the parent Fc region.

In one aspect, it is preferable that a variant Fc region of the present disclosure does not have a substantially increased FcRn binding activity, especially at pH7.4, compared to the parent Fc region.

“FcRn” is structurally similar to polypeptides of major histocompatibility complex (MHC) class I, and exhibits 22% to 29% sequence identity with MHC class I molecules. FcRn is expressed as a heterodimer consisting of a soluble beta or light chain (beta 2 microglobulin) complexed with a transmembrane alpha or heavy chain. Like MHC, the alpha chain of FcRn contains three extracellular domains (alpha1, alpha2, and alpha3), and its short cytoplasmic domain tethers them to the cell surface. The alpha1 and alpha2 domains interact with the FcRn-binding domain of the antibody Fc region. The polynucleotide and amino acid sequences of human FcRn may be derived, for example, from the precursors shown in NM_004107.4 and NP_004098.1 (containing the signal sequence), respectively.

The amino acid sequence of human FcRn (alpha chain) is set forth in SEQ ID NO:

46; and the amino acid sequence of human beta2 microglobulin is set forth in SEQ ID NO: 47.

In one aspect, it is preferable that a variant Fc region of the present disclosure does not have a substantially increased FcRn binding activity, especially at pH7.4, that is less than 1000 fold, less than 500 fold, less than 200 fold, less than 100 fold, less than 90 fold, less than 80 fold, less than 70 fold, less than 60 fold, less than 50 fold, less than 40 fold, less than 30 fold, less than 20 fold, less than 10 fold, less than 5 fold, less than 3 fold, or less than 2 fold compared to the FcRn binding activity for the parent Fc region. In one aspect, a variant Fc region of the present disclosure does not have a substantially increased FcRn binding activity, especially at pH7.4, which means that the ratio of [the difference in the RU values of sensorgrams that changed before and after interaction of FcRn with the variant Fc region]/[the difference in the RU values of sensorgrams that changed before and after capturing FcRn to the sensor chips] is less than 0.5, less than 0.3, less than 0.2, less than 0.1, less than 0.08, less than 0.05, less than 0.03, less than 0.02, less than 0.01, less than 0.008, less than 0.005, less than 0.003, less than 0.002, or less than 0.001.

In another aspect, the variant Fc region of the present disclosure can further comprise at least one amino acid alteration of at least one position selected from the group consisting of: 428, 434, 436, 438, and 440, according to EU numbering.

In a further aspect, the variant Fc region can further comprise amino acids selected from the group consisting of: (a) Ala at position 434; (b) Ala at position 434, Thr at position 436, Arg at position 438, and Glu at position 440; (c) Leu at position 428, Ala at position 434, Thr at position 436, Arg at position 438, and Glu at position 440; (d) Leu at position 428 and Ala at position 434; and (e) Leu at position 428, Ala at position 434, Arg at position 438, and Glu at position 440, according to EU numbering (see also WO2016/125495 describing a relationship between amino acid alterations and binding activity of a variant Fc region).

In another aspect, the variant Fc region of the present disclosure comprises amino acids of: Ala at position 234, Ala at position 235, Ala at position 326, Ser at position 333, Leu at position 428, Ala at position 434, Thr at position 436, Arg at position 438, and Glu at position 440, according to EU numbering. In another aspect, the variant Fc region of the present disclosure comprises amino acids of: Ala at position 234, Ala at position 235, Ala at position 326, Ser at position 333, Leu at position 428, Ala at position 434, Arg at position 438, and Glu at position 440, according to EU numbering.

In another aspect, the variant Fc region of the present disclosure comprises any of the amino acid alterations, singly or in combination, described in Table 1 below (see also, e.g., WO2018/052375). In another aspect, the variant Fc region of the present disclosure comprises at least any one of the amino acid alterations described in Table 1.

TABLE 1

| | | | SEQ |
|---|---|---|---|
| Variant name | ID | Mutations | ID NO: |
| WT | SG182 | — | 48 |
| LALA | SG192 | L234A, L235A | 1 |
| KWES | SG1085 | K326W, E333S | 49 |
| EFT + AE | SG1086 | G236A, S267E, H268F, S324T, I332E | 50 |
| EFT | SG1087 | S267E, H268F, S324T | 51 |
| LALA + KWES | SG1088 | L234A, L235A, K326W, E333S | 52 |
| LALA + EFT + | SG1089 | L234A, L235A, G236A, S267E, H268F, | 53 |
| AE | | S324T, I332E | |
| LALA + EFT | SG1090 | L234A, L235A, S267E, H268FM S324T | 54 |
| LALA + KAES | SG1095 | L234A, L235A, K326A, E333S | 2 |
| LALA + KDES | SG1096 | L234A, L235A, K326D, E333S | 55 |
| LALA + KEES | SG1097 | L234A, L235A, K326F, E333S | 56 |
| LALA + KMES | SG1098 | L234A, L235A, K326M, E333S | 57 |
| LALA + ACT3 + | SG1105 | L234A, L235A, K326A, E333S, M428L, | 58 |
| KAES | | N434A, Y436T, Q438R, S440E | |
| LALA + ACT5 + | SG1106 | L234A, L235A, K326A, E333S, M428L, | 59 |
| KAES | | N434A, Q438R, S440F | |
| KAES | SG1109 | K326A, E333S | 60 |
| LALA + ACT3 | SG1044 | L234A, L235A, M428L, N434AM Y436T, | 61 |
| | | Q438R, S440E | |
| LALA + ACT5 | SG1045 | L234A, L235A, M428L, N434A, Q438R, | 62 |
| | | S440E | |

Two or more variant Fc regions described herein can be included in one Fc polypeptide, wherein two variant Fc regions are associated, much like in an antibody. The type of antibody is not limited, and IgA (IgA1, IgA2), IgD, IgE, IgG (IgG1, IgG2, IgG3, IgG4), and IgM, or such can be used.

As described above, the antigen-binding molecule of the present disclosure comprises the first and the second variant Fc regions. In one embodiment, the antigen-binding molecule of the present disclosure is a one-armed antibody. In certain embodiments, a one-armed antibody is a chimeric antibody, or a humanized antibody. The origin of a one-armed antibody is not particularly limited, but examples include a human antibody, a mouse antibody, a rat antibody, and a rabbit antibody. In a further embodiment, the antigen-binding polypeptide is an Fc fusion protein. In further embodiments, an antigen-binding molecule comprising an Fc polypeptide of the present disclosure is an anti-virus one-armed antibody.

In one aspect, the present disclosure provides an antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen.

In one aspect, the antigen-binding molecule of the present disclosure has a substantially decreased Fc gamma R-binding activity. In one aspect, the antigen-binding molecule of the present disclosure has a maintained (not substantially decreased) or increased C1q-binding activity.

In one aspect, the antigen-binding molecule of the present disclosure has a substantially decreased Fc gamma R-binding activity and has a maintained (not substantially decreased) or increased C1q-binding activity.

In one aspect, the present disclosure provides an antigen-binding molecule comprising the Fc polypeptide which is:
(i) an Fc polypeptide which exhibits reduced binding affinity to human Fc gamma receptor, as compared to a parent Fc polypeptide comprising parent native human IgG1 Fc regions,
wherein the first and/or the second Fc region variants comprised in the Fc polypeptide comprises (f1) or (f2) below:
(f1) Ala at position 234 and Ala at position 235;
(f2) Ala at position 234, Ala at position 235, and Ala at position 297;
wherein the amino acid positions are numbered according to EU index.

In one aspect, the present disclosure provides an antigen-binding molecule comprising the Fc polypeptide which is:
(i) an Fc polypeptide which exhibits reduced binding affinity to human Fc gamma receptor, as compared to a parent Fc polypeptide comprising parent native human IgG1 Fc regions,
wherein the Fc polypeptide further exhibits maintained (not substantially decreased) or increased C1q-binding activity as compared to the parent Fc polypeptide comprising the parent native human IgG1 Fc regions,
wherein the first and/or the second Fc region variants comprised in the Fc polypeptide comprises (f1) or (f2) below:
(f1) Ala at position 234 and Ala at position 235;
(f2) Ala at position 234, Ala at position 235, and Ala at position 297; and
wherein the first and/or the second Fc region variants comprised in the Fc polypeptide further comprises amino acids selected from the group consisting of (f3) to (f9) below:
(f3) Glu at position 267;
(f4) Phe at position 268;
(f5) Thr at position 324;
(f6) Ala at position 236;
(f7) Glu at position 332;
(f8) Ala, Asp, Glu, Met, or Trp at position 326; and
(f9) Ser at position 333;
wherein the amino acid positions are numbered according to EU index.

In one aspect, the present disclosure provides an antigen-binding molecule comprising the Fc polypeptide which is:
(i) an Fc polypeptide which exhibits reduced binding affinity to human Fc gamma receptor, as compared to a parent Fc polypeptide comprising parent native human IgG1 Fc regions,
wherein the Fc polypeptide further exhibits maintained (not substantially decreased) or increased C1q-binding activity as compared to the parent Fc polypeptide comprising the parent native human IgG1 Fc regions, and
wherein the Fc polypeptide further exhibits stronger FcRn binding affinity to human FcRn under acidic condition, as compared to the parent Fc polypeptide.

In one aspect, the present disclosure provides an antigen-binding molecule comprising the Fc polypeptide which is:
(i) an Fc polypeptide which exhibits reduced binding affinity to human Fc gamma receptor, as compared to a parent Fc polypeptide comprising parent native human IgG1 Fc regions,
   wherein the Fc polypeptide further exhibits maintained (not substantially decreased) or increased C1q-binding activity as compared to the parent Fc polypeptide comprising the parent native human IgG1 Fc regions,
   wherein the Fc polypeptide further exhibits stronger FcRn binding affinity to human FcRn under acidic condition, as compared to the parent Fc polypeptide,
   wherein the first and/or the second Fc region variants comprised in the Fc polypeptide comprises, in addition to (f1) or (f2) above and amino acids selected from the group consisting of (f3) to (f9) above, Leu at position 428, Ala at position 434, Thr at position 436, Arg at position 438, and/or Glu at position 440, and
wherein the amino acid positions are numbered according to EU index.

In addition, amino acid alterations performed for other purpose(s) can be combined in a variant Fc region described herein.

For example, in addition to amino acid alterations at positions 234 and 235, an amino acid substitution at a position selected from the group of E233, N297, P331, and P329 may be introduced to reduce the binding affinity of an Fc region to an Fc gamma receptor. In one embodiment, the variant Fc region comprises an amino acid substitution at position P329. In a more specific embodiment, the amino acid substitution is P329A or P329G, particularly P329G. In one embodiment the variant Fc region comprises an amino acid substitution at position P329 and a further amino acid substitution at a position selected from E233, L234, L235, N297, and P331. In a more specific embodiment, the further amino acid substitution is E233P, L234A, L235A, L235E, N297A, N297D, or P331S. In particular embodiments, the Fc domain comprises amino acid substitutions at positions P329, L234, and L235. In more particular embodiments the Fc domain comprises the amino acid mutations L234A, L235A, and P329G (“P329G LALA”). In one such embodiment, the Fc domain is an IgG1 Fc domain, particularly a human IgG1 Fc domain. The “P329G LALA” combination of amino acid substitutions almost completely abolishes Fc gamma receptor (as well as complement) binding of a human IgG1 Fc domain, as described in PCT publication no. WO 2012/130831. WO 2012/130831 also describes methods of preparing such mutant Fc domains and methods for determining its properties such as Fc receptor binding or effector functions.

In certain embodiments, N-glycosylation of the Fc region has been eliminated. In one such embodiment, the Fc region comprises an amino acid mutation at position N297, particularly an amino acid substitution replacing asparagine by alanine (N297A) or aspartic acid (N297D).

In a particular embodiment, the variant Fc region exhibiting reduced binding affinity to an Fc receptor, as compared to a native IgG1 Fc domain, is a human IgG1 Fc region comprising the amino acid substitutions L234A, L235A, and N297A.

For example, amino acid substitutions that improve FcRn-binding activity (Hinton et al., J. Immunol. 176(1):346-356 (2006); Dall'Acqua et al., J. Biol. Chem. 281(33):23514-23524 (2006); Petkova et al., Intl. Immunol. 18(12):1759-1769 (2006); Zalevsky et al., Nat. Biotechnol. 28(2):157-159 (2010); WO 2006/019447; WO 2006/053301; and WO 2009/086320), and amino acid substitutions for improving antibody heterogeneity or stability (WO 2009/041613) may be added. Alternatively, polypeptides with the property of promoting antigen clearance, which are described in WO 2011/122011, WO 2012/132067, WO 2013/046704 or WO 2013/180201, polypeptides with the property of specific binding to a target tissue, which are described in WO 2013/180200, polypeptides with the property for repeated binding to a plurality of antigen molecules, which are described in WO 2009/125825, WO 2012/073992 or WO 2013/047752, can be combined with a variant Fc region described herein. Alternatively, with the objective of conferring binding ability to other antigens, the amino acid alterations disclosed in EP1752471 and EP1772465 may be combined in CH3 of a variant Fc region described herein. Alternatively, with the objective of increasing plasma retention, amino acid alterations that decrease the pI of the constant region (WO 2012/016227) may be combined in a variant Fc region described herein. Alternatively, with the objective of promoting uptake into cells, amino acid alterations that increase the pI of the constant region (WO 2014/145159) may be combined in a variant Fc region described herein. Alternatively, with the objective of promoting elimination of a target molecule from plasma, amino acid alterations that increase the pI of the constant region (WO2016/125495 and WO2016/098357) may be combined in a variant Fc region described herein.

Amino acid alterations of enhancing human FcRn-binding activity under acidic pH can also be combined in a variant Fc region described herein. Specifically, such alterations may include, for example, substitution of Leu for Met at position 428 and substitution of Ser for Asn at position 434, according to EU numbering (Nat Biotechnol, 2010, 28: 157-159); substitution of Ala for Asn at position 434 (Drug Metab Dispos, 2010 April; 38(4): 600-605); substitution of Tyr for Met at position 252, substitution of Thr for Ser at position 254 and substitution of Glu for Thr at position 256 (J Biol Chem, 2006, 281: 23514-23524); substitution of Gln for Thr at position 250 and substitution of Leu for Met at position 428 (J Immunol, 2006, 176(1): 346-356); substitution of His for Asn at position 434 (Clin Pharmacol Ther, 2011, 89(2): 283-290), and alterations described in WO2010/106180, WO2010/045193, WO2009/058492, WO2008/022152, WO2006/050166, WO2006/053301, WO2006/031370, WO2005/123780, WO2005/047327, WO2005/037867, WO2004/035752, WO2002/060919, or such. In another embodiment, such alterations may include, for example, at least one alteration selected from the group consisting of substitution of Leu for Met at position 428, substitution of Ala for Asn at position 434 and substitution of Thr for Tyr at position 436. Those alterations may further include substitution of Arg for Gln at position 438 and/or substitution of Glu for Ser at position 440 (WO2016/125495).

In one embodiment, the antigen-binding molecule of the present disclosure comprising the variant Fc regions with modified effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In one embodiment, the antigen-binding molecule of the present disclosure comprising the variant Fc regions may have altered binding to FcRs as described. (See, e.g., U.S. Pat. No. 6,737,056; WO 2004/056312, and Shields et al., J. Biol. Chem. 9(2): 6591-6604 (2001).)

In certain embodiments, the antigen-binding molecule of the present disclosure comprises an Fc region with one or more amino acid substitutions which alter ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, WO2011/091078, and Idusogie et al. J. Immunol. 164: 4178-4184 (2000).

In one embodiment, the antigen-binding molecule of the present disclosure with increased half-lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In another embodiment, an antigen-binding molecule may comprise an Fc region variant of the present disclosure described herein below in detail.

In some embodiments, the Fc polypeptide of the antigen-binding molecule consists of a pair of Fc regions or Fc domains, which are polypeptide chains comprising heavy chain domains of an immunoglobulin molecule. For example, the Fc polypeptide of an immunoglobulin G (IgG) molecule is a dimer, each subunit of which comprises the CH2 and CH3 IgG heavy chain constant domains. The two subunits of the Fc polypeptide are capable of stable association with each other. In one embodiment the antigen-binding molecule described herein comprises not more than one Fc polypeptide.

In one embodiment described herein, the Fc polypeptide of the antigen binding molecule is an IgG Fc polypeptide. In a particular embodiment, the Fc polypeptide is an IgG1 Fc polypeptide. In another embodiment, the Fc polypeptide is an IgG1 Fc polypeptide. In a further particular embodiment, the Fc polypeptide is a human IgG1 Fc polypeptide.

In some embodiments, an antigen-binding molecule comprising an Fc polypeptide of the present invention is a one-armed antibody or an Fc fusion protein comprising a domain(s) which can bind to an antigen. Examples of antigens that can be bound by such antibodies and Fc fusion proteins include, but are not limited to ligands (cytokines, chemokines, and such), receptors, cancer antigens, viral antigens, MHC antigens, differentiation antigens, immunoglobulins, and immune complexes partly containing immunoglobulins. In one non-limiting embodiment, the antigen-binding molecule of the present disclosure specifically binds to a virus which can cause the risk of Antibody-Dependent Enhancement (ADE) when the antigen-binding molecule is administered.

In some embodiments, each of the first Fc region variant and the second Fc variant of the present disclosure comprises at least one amino acid alteration promoting the association of the first Fc region variant and the second Fc region variant.

Techniques for promoting the association among H chains and between L and H chains having the desired combinations can be applied to the association of the first Fc region variant and the second Fc variant of the present disclosure.

In certain embodiments, the antigen-binding molecule of the disclosure can be secreted from a hybrid hybridoma (quadroma) produced by fusing two types of hybridomas that produce IgG antibodies (Milstein et al., Nature (1983) 305, 537-540).

In certain embodiments, techniques for suppressing undesired H-chain association by introducing electrostatic repulsion at the interface of the second constant region or the third constant region of the antibody H chain (CH2 or CH3) to produce bispecific antibodies efficiently, can be applied to the association of the first Fc region variant and the second Fc variant of the present disclosure (see, e.g., WO2006/106905).

In the technique of suppressing unintended H-chain association by introducing electrostatic repulsion at the interface of CH2 or CH3, examples of amino acid residues in contact at the interface of the other constant region of the H chain include regions corresponding to the residues at EU numbering positions 356, 439, 357, 370, 399, and 409 in the CH3 region.

More specifically, examples include an antibody comprising two types of H-chain CH3 regions, in which one to three pairs of amino acid residues in the first H-chain CH3 region, selected from the pairs of amino acid residues indicated in (1) to (3) below, carry the same type of charge: (1) amino acid residues comprised in the H chain CH3 region at EU numbering positions 356 and 439; (2) amino acid residues comprised in the H-chain CH3 region at EU numbering positions 357 and 370; and (3) amino acid residues comprised in the H-chain CH3 region at EU numbering positions 399 and 409.

Furthermore, the antibody may be an antibody in which pairs of the amino acid residues in the second H-chain CH3 region which is different from the first H-chain CH3 region mentioned above, are selected from the aforementioned pairs of amino acid residues of (1) to (3), wherein the one to three pairs of amino acid residues that correspond to the aforementioned pairs of amino acid residues of (1) to (3) carrying the same type of charges in the first H-chain CH3 region mentioned above carry opposite charges from the corresponding amino acid residues in the first H-chain CH3 region mentioned above.

Each of the amino acid residues indicated in (1) to (3) above come close to each other during association. Those skilled in the art can find out positions that correspond to the above-mentioned amino acid residues of (1) to (3) in a desired H-chain CH3 region or H-chain constant region by homology modeling and such using commercially available software, and amino acid residues of these positions can be appropriately subjected to modification.

In the antibodies mentioned above, “charged amino acid residues” are preferably selected, for example, from amino acid residues included in either one of the following groups:
(a) glutamic acid (E) and aspartic acid (D); and
(b) lysine (K), arginine (R), and histidine (H).

In the above-mentioned antibodies, the phrase “carrying the same charge” means, for example, that all of the two or more amino acid residues are selected from the amino acid residues included in either one of groups (a) and (b) mentioned above. The phrase “carrying opposite charges” means, for example, that when at least one of the amino acid residues among two or more amino acid residues is selected from the amino acid residues included in either one of groups (a) and (b) mentioned above, the remaining amino acid residues are selected from the amino acid residues included in the other group.

In a preferred embodiment, the antibodies mentioned above may have their first H-chain CH3 region and second H-chain CH3 region crosslinked by disulfide bonds.

In the present invention, amino acid residues subjected to modification are not limited to the above-mentioned amino acid residues of the antibody variable regions or the antibody constant regions. Those skilled in the art can identify the amino acid residues that form an interface in mutant polypeptides or heteromultimers by homology modeling and such using commercially available software; and amino acid residues of these positions can then be subjected to modification so as to regulate the association.

Other known techniques can also be used for the association of the first Fc region variant and the second Fc variant of the present disclosure. Fc region-containing polypeptides comprising different amino acids can be efficiently associated with each other by substituting an amino acid side chain present in one of the H-chain Fc regions of the antibody with a larger side chain (knob), and substituting an amino acid side chain present in the corresponding Fc region of the other H chain with a smaller side chain (hole) to allow placement of the knob within the hole (WO1996/027011; Ridgway J B et al., Protein Engineering (1996) 9, 617-621; Merchant A. M. et al. Nature Biotechnology (1998) 16, 677-681; and US20130336973).

In addition, other known techniques can also be used for the association of the first Fc region variant and the second Fc variant of the present disclosure. Association of polypeptides having different sequences can be induced efficiently by complementary association of CH3 using a strand-exchange engineered domain CH3 produced by changing part of one of the H-chain CH3s of an antibody to a corresponding IgA-derived sequence and introducing a corresponding IgA-derived sequence into the complementary portion of the other H-chain CH3 (Protein Engineering Design & Selection, 23; 195-202, 2010). This known technique can also be used to efficiently form bispecific antibodies of interest.

In addition, technologies for antibody production using association of antibody CH1 and CL and association of VH and VL as described in WO 2011/028952, WO2014/018572, and Nat Biotechnol. 2014 February; 32(2):191-8; technologies for producing bispecific antibodies using separately prepared monoclonal antibodies in combination (Fab Arm Exchange) as described in WO2008/119353 and WO2011/131746; technologies for regulating association between antibody heavy-chain CH3s as described in WO2012/058768 and WO2013/063702; technologies for producing bispecific antibodies composed of two types of light chains and one type of heavy chain as described in WO2012/023053; technologies for producing bispecific antibodies using two bacterial cell strains that individually express one of the chains of an antibody comprising a single H chain and a single L chain as described by Christoph et al. (Nature Biotechnology Vol. 31, p 753-758 (2013)); and such may be used for the formation of the antigen-binding molecules of the present disclosure.

Alternatively, even when an antigen-binding molecule of interest cannot be formed efficiently, an antigen-binding molecule of the present invention can be obtained by separating and purifying the antigen-binding molecule of interest from the produced molecules. For example, a method for enabling purification of two types of homomeric forms and the heteromeric molecule of interest by ion-exchange chromatography by imparting a difference in isoelectric points by introducing amino acid substitutions into the variable regions of the two types of H chains has been reported (WO2007114325). To date, as a method for purifying heteromeric antigen-binding molecules, methods using Protein A to purify a heterodimeric antibody comprising a mouse IgG2a H chain that binds to Protein A and a rat IgG2b H chain that does not bind to Protein A have been reported (WO98050431 and WO95033844). Furthermore, a heterodimeric antigen-binding molecule can be purified efficiently on its own by using H chains comprising substitution of amino acid residues at EU numbering positions 435 and 436, which is the IgG-Protein A binding site, with Tyr, His, or such which are amino acids that yield a different Protein A affinity, or using H chains with a different protein A affinity, to change the interaction of each of the H chains with Protein A, and then using a Protein A column.

In one aspect, the present disclosure provides an antigen-binding molecule comprising the Fc polypeptide which is:
(i) an Fc polypeptide which exhibits reduced binding affinity to human Fc gamma receptor, as compared to a parent Fc polypeptide comprising parent native human IgG1 Fc regions,
   wherein the Fc polypeptide is composed of a first Fc region variant and a second Fc region variant that are capable of stable association.

In one aspect, the present disclosure provides an antigen-binding molecule comprising the Fc polypeptide which is:
(i) an Fc polypeptide which exhibits reduced binding affinity to human Fc gamma receptor, as compared to a parent Fc polypeptide comprising parent native human IgG1 Fc regions,
wherein the Fc polypeptide comprises (e1) or (e2) below:
(e1) the first Fc region variant comprising Cys at position 349, Ser at position 366, Ala at position 368 and Val at position 407, and the second Fc region variant comprising Cys at position 354 and Trp at position 366;
(e2) the first Fc region variant comprising Glu at position 439, and the second Fc region variant comprising Lys at position 356;
wherein the amino acid positions are numbered according to EU index.

In one aspect, the present disclosure provides an antigen-binding molecule comprising:
(i) a first antigen-binding moiety which specifically binds to an antigen, and
(ii) an Fc polypeptide,
   wherein the Fc polypeptide comprises a first Fc region variant and a second Fc region variant comprising at least one amino acid alteration relative to a parent Fc region, wherein the first Fc variant region is fused to the first antigen-binding moiety, provided that the second Fc region variant is not fused to the first antigen-binding moiety or to any other antigen-binding moieties which specifically binds to the antigen, and wherein the antigen-binding molecule has a substantially decreased Fc gamma receptor-binding activity and has a maintained or increased C1 q-binding activity when compared to an antigen binding molecule comprising the parent Fc region.

In one embodiment, the first and second variant Fc regions of the present disclosure comprise at least one amino acid alteration that enhances hexamer formation.

The techniques for enhancing hexamer formation are generally well understood and commonly employed using conventional methodology by those skilled in the art, (See, e.g., WO2018/224609, WO2018/146317, WO2016/164480, WO2013/004842, WO2014/108198, WO2014/006217, WO2018/031258).

In certain embodiments, the antigen-binding molecule of the present disclosure comprises an Fc region with one or more amino acid substitutions which can enhance hexamer formation, e.g., substitutions E345R, E430G, S440Y, K248E, and/or T437R of the Fc region (EU numbering of residues).

In certain embodiments, the present disclosure provides an antigen-binding molecule comprising the Fc polypeptide, wherein the first and/or the second Fc region variants comprised in the Fc polypeptide comprises amino acids selected from the group consisting of (a1) to (a5) below:
(a1) Arg at position 345;
(a2) Gly at position 430;
(a3) Tyr at position 440;
(a4) Glu at position 248; and
(a5) Arg at position 437;
wherein the amino acid positions are numbered according to EU index.

In certain embodiments, the present disclosure provides an antigen-binding molecule comprising the Fc polypeptide, wherein each of the first Fc region variant and the second Fc region variant comprised the Fc polypeptide comprises amino acids selected from the group consisting of:
(a) Arg at position 345;
(b) Arg at position 345, and Gly at position 430;
(c) Arg at position 345, Gly at position 430, and Tyr at position 440; and
(d) Glu at position 248, and Arg at position 437;
according to EU numbering.

In the present invention, amino acid alteration means any of substitution, deletion, addition, insertion, and modification, or a combination thereof. In the present invention, amino acid alteration may be rephrased as amino acid mutation.

Amino acid alterations are produced by various methods known to those skilled in the art. Such methods include the site-directed mutagenesis method (Hashimoto-Gotoh et al., Gene 152:271-275 (1995); Zoller, Meth. Enzymol. 100:468-500 (1983); Kramer et al., Nucleic Acids Res. 12: 9441-9456 (1984)); Kramer and Fritz, Methods Enzymol. 154: 350-367 (1987); and Kunkel, Proc. Natl. Acad. Sci. USA 82:488-492 (1985)), the PCR mutation method, and the cassette mutation method, but are not limited thereto.

The number of amino acid alterations introduced into an Fc region is not limited. In certain embodiments, it can be 1, 2 or less, 3 or less, 4 or less, 5 or less, 6 or less, 8 or less, 10 or less, 12 or less, 14 or less, 16 or less, 18 or less, or 20 or less.

Furthermore, an antigen-binding molecule comprising a variant Fc region of the present invention may be chemically modified with various molecules such as polyethylene glycol (PEG) and cytotoxic substances. Methods for such chemical modification of a polypeptide are established in the art.

The antigen-binding molecule of the present disclosure comprising the first and the second Fc region variants has a substantially decreased Fc gamma receptor-binding activity, and/or has a maintained (does not have a substantially decreased) C1q-binding activity or increased C1q-binding activity, and/or has an increased FcRn binding activity under acidic pH, and/or does not have a substantially increased FcRn binding activity at neutral pH, when compared to an antigen-binding molecule comprising the parent Fc region or polypeptide.

In one embodiment, the “antigen-binding molecule comprising the parent Fc region” is an one-armed antigen-binding molecule which comprises the same first antigen-binding moiety as the antigen-binding molecule of the present disclosure and a “parent” Fc polypeptide which is composed of two parent Fc regions. In the “antigen-binding molecule comprising the parent Fc region”, one out of the two parent Fc regions is fused to the first antigen-binding moiety but the other of the two parent Fc regions is not fused to the first antigen-binding moiety or to any other antigen-binding moieties which specifically bind to the antigen. As described above, the “parent” Fc polypeptide is composed of two parent Fc regions having the amino acid sequence identical to the first and second Fc region variants except for the amino acid alteration(s) described herein. When compared to the “antigen binding molecule comprising the parent Fc region”, the antigen-binding molecule of the present disclosure (comprising (i) the first antigen-binding moiety and (ii) an Fc polypeptide which comprises a first Fc region variant and a second Fc region variant each comprising at least one amino acid alteration relative to the parent Fc region) has a substantially decreased Fc gamma receptor-binding activity, and/or has a maintained or increased C1q-binding activity, and/or has an increased FcRn binding activity under acidic pH, and/or does not have a substantially increased FcRn binding activity at neutral pH.

In another embodiment, the “antigen-binding molecule comprising the parent Fc region” is a conventional two-armed antigen-binding molecule which comprises two first antigen-binding moieties and a parent Fc polypeptide which is composed of two parent Fc regions. One out of the two parent Fc regions is fused to one out of the two first antigen-binding moieties and the other of the two parent Fc regions is fused to the other of the two first antigen-binding moieties. As described above, the “parent” Fc polypeptide is composed of two parent Fc regions having the amino acid sequence identical to the first and second Fc region variants except for the amino acid alteration(s) described herein. When compared to the “antigen binding molecule comprising the parent Fc region”, the antigen-binding molecule of the present disclosure (comprising (i) the first antigen-binding moiety and (ii) an Fc polypeptide which comprises a first Fc region variant and a second Fc region variant each comprising at least one amino acid alteration in the parent Fc region) has a substantially decreased Fc gamma receptor-binding activity, and/or has a maintained or increased C1q-binding activity, and/or has an increased FcRn binding activity under acidic pH, and/or does not have a substantially increased FcRn binding activity at neutral pH.

### Method for Producing an Antigen-Binding Molecule

Furthermore, the present invention provides a method for producing an antigen-binding molecule comprising a Fc polypeptide comprising a first and a second variant Fc regions (or first and second Fc region variants, collectively referred to herein as a “Fc region variant(s)” or “variant Fc region(s)”)) with a substantially decreased Fc gamma receptor-binding activity and without a substantially decreased C1q-binding activity in comparison with an antigen-binding molecule comprising a parent Fc region, which method comprises introducing at least one amino acid alteration to the parent Fc region. In some aspects, the produced antigen-binding molecule is a one-armed antibody. In certain embodiments, a one-armed antibody is a chimeric antibody, or a humanized antibody. In some aspects, the produced antigen-binding molecule is an Fc fusion protein.

In one aspect, in the above-mentioned method for producing an antigen-binding molecule comprising variant Fc regions with a substantially decreased Fc gamma receptor-binding activity and without a substantially decreased C1q-binding activity, at least one amino acid is altered at at least one position selected from the group consisting of: 234, 235, 236, 267, 268, 324, 326, 332, and 333, according to EU numbering.

In another aspect, in the above-mentioned method for producing an antigen-binding molecule comprising variant Fc regions with a substantially decreased Fc gamma receptor-binding activity and without a substantially decreased C1q-binding activity, two amino acids are altered at positions 234 and 235.

In another aspect, in the above-mentioned method for producing an antigen-binding molecule comprising variant Fc regions with a substantially decreased Fc gamma receptor-binding activity and without a substantially decreased C1q-binding activity, amino acids are altered and the alterations comprise: (a) two amino acid alterations at positions 234 and 235, and (b) at least one amino acid alteration of at least one position selected from the group consisting of: 236, 267, 268, 324, 326, 332, and 333, according to EU numbering.

In another aspect, in the above-mentioned method for producing an antigen-binding molecule comprising variant Fc regions with a substantially decreased Fc gamma receptor-binding activity and without a substantially decreased C1q-binding activity, amino acids are altered and the alterations comprise: (a) two amino acid alterations at positions 234 and 235, and (b) at least one amino acid alterations of any one of the following (i)-(iii): (i) positions 267, 268, and 324; (ii) positions 236, 267, 268, 324, and 332; and (iii) positions 326 and 333, according to EU numbering.

In a further aspect, in the above-mentioned method for producing an antigen-binding molecule comprising variant Fc regions with a substantially decreased Fc gamma receptor-binding activity and without a substantially decreased C1q-binding activity, one or more amino acids are altered so that the variant Fc regions comprise an altered amino acid selected from the group consisting of: (a) Ala at position 234; (b) Ala at position 235; (c) Glu at position 267; (d) Phe at position 268; (e) Thr at position 324; (f) Ala at position 236; (g) Glu at position 332; (h) Ala, Asp, Glu, Met, Trp at position 326; and (i) Ser at position 333, according to EU numbering.

In a further aspect, in the above-mentioned method for producing an antigen-binding molecule comprising variant Fc regions with a substantially decreased Fc gamma receptor-binding activity and without a substantially decreased C1q-binding activity, amino acid alterations are Ala at position 234, Ala at position 235, Ala at position 326, and Ser at position 333, according to EU numbering.

In a further aspect, in the above-mentioned method for producing an antigen-binding molecule comprising variant Fc regions with a substantially decreased Fc gamma receptor-binding activity and without a substantially decreased C1q-binding activity, amino acid alterations are Ala at position 234, Ala at position 235, Asp at position 326, and Ser at position 333, according to EU numbering.

In a further aspect, in the above-mentioned method for producing an antigen-binding molecule comprising variant Fc regions with a substantially decreased Fc gamma receptor-binding activity, and without a substantially decreased C1q-binding activity, amino acid alterations are Ala at position 234, Ala at position 235, Glu at position 326, and Ser at position 333, according to EU numbering.

In a further aspect, in the above-mentioned method for producing an antigen-binding molecule comprising variant Fc regions with a substantially decreased Fc gamma receptor-binding activity, and without a substantially decreased C1q-binding activity, amino acid alterations are Ala at position 234, Ala at position 235, Met at position 326, and Ser at position 333, according to EU numbering.

In a further aspect, in the above-mentioned method for producing an antigen-binding molecule comprising variant Fc regions with a substantially decreased Fc gamma receptor-binding activity, and without a substantially decreased C1q-binding activity, amino acid alterations are Ala at position 234, Ala at position 235, Trp at position 326, and Ser at position 333, according to EU numbering.

In another aspect, at least one amino acid is further altered in the above-mentioned method, that further alteration is made at at least one position selected from the group consisting of 428, 434, 436, 438, and 440, according to EU numbering.

In a further aspect, the above-described further amino acid alteration(s) in the above-mentioned method is the alteration(s) to the set of amino acids selected from the following (a)-(d): (a) Ala at position 434; (b) Ala at position 434, Thr at position 436, Arg at position 438, and Glu at position 440; (c) Leu at position 428, Ala at position 434, Thr at position 436, Arg at position 438, and Glu at position 440; (d) Leu at position 428 and Ala at position 434; and (e) Leu at position 428, Ala at position 434, Arg at position 438, and Glu at position 440, according to EU numbering (see also WO2016/125495 describing a relationship between amino acid alterations and FcRn-binding activity of a variant Fc region).

In a further aspect, amino acid alterations in the above-mentioned method are Ala at position 234, Ala at position 235, Ala at position 326, Ser at position 333, Leu at position 428, Ala at position 434, Thr at position 436, Arg at position 438, and Glu at position 440, according to EU numbering.

In a further aspect, amino acid alterations in the above-mentioned method are Ala at position 234, Ala at position 235, Ala at position 326, Ser at position 333, Leu at position 428, Ala at position 434, Arg at position 438, and Glu at position 440, according to EU numbering.

In one aspect, it is preferable that a variant Fc region or a variant Fc polypeptide comprising a first and a second Fc region variants of the invention does not have a substantially increased FcRn binding activity, especially at pH7.4, compared to the parent Fc region.

In a further aspect, the amino acid alterations in the above-mentioned production methods are selected from any single alteration, combination of single alterations, or combination alterations described in Table 1.

Antigen-binding molecules comprising a variant Fc region produced by any of the above-mentioned methods or other methods know in the art are included in the present invention.

In one aspect, assays are provided for identifying antigen-binding molecules comprising variant Fc regions having biological activity. Biological activity may include, e.g., ADCC activity and CDC activity. Antigen-binding molecules comprising variant Fc regions having such biological activity in vivo and/or in vitro are also provided.

In certain embodiments, an antigen-binding molecule comprising a variant Fc region of the invention is tested for such biological activity. In a certain aspect, an antigen-binding molecule comprising a variant Fc region of the invention modulate an effector function as compared to the polypeptide comprising a parent Fc region. In a certain aspect, this modulation is a modulation of ADCC and/or CDC.

In vitro and/or in vivo cytotoxicity assays can be conducted to confirm the CDC and/or ADCC activities. For example, Fc receptor (FcR) binding assays can be conducted to check if the antibody has Fc gamma receptor binding (hence likely having ADCC activity), and retains FcRn binding ability. The primary cells for mediating ADCC, NK cells express Fc gamma receptor III only, whereas monocytes express Fc gamma receptor I, Fc gamma receptor II and Fc gamma receptor III. FcR expression on hematopoietic cells is summarized in Table 3 on page 464 of Ravetch and Kinet, Annu Rev Immunol (1991) 9, 457-492. Non-limiting examples of in vitro assays to assess ADCC activity of a molecule of interest is described in U.S. Pat. No. 5,500,362 (see, e.g. Hellstrom et al, Proc Natl Acad Sci USA (1986) 83, 7059-7063) and Hellstrom et al, Proc Natl Acad Sci USA (1985) 82, 1499-1502; U.S. Pat. No. 5,821,337 (see Bruggemann et al, J Exp Med (1987) 166, 1351-1361). Alternatively, non-radioactive assay methods may be employed (see, for example, ACTI™ non-radioactive cytotoxicity assay for flow cytometry (CellTechnology, Inc. Mountain View, Calif.); and CytoTox 96 (registered trademark) non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al, Proc Natl Acad Sci USA (1998) 95, 652-656. C1q binding assays may also be carried out to confirm whether the antibody binds C1q and hence has CDC activity. See, e.g., C1q and C3c binding ELISA in WO 2006/029879 and WO 2005/100402. To assess complement activation, a CDC assay may be performed (see, for example, Gazzano-Santoro et al, J Immunol Methods (1997) 202, 163-171; Cragg et al, Blood (2003) 101, 1045-1052; and Cragg and Glennie, Blood (2004) 103, 2738-2743). FcRn binding and in vivo clearance/half-life determinations can also be performed using methods known in the art (see, e.g., Petkova et al, Int Immunol (2006) 18, 1759-1769).

In one embodiment, the present disclosure provides a method for producing an antigen-binding molecule, which comprises the steps of:
(a) selecting an antigen-binding molecule that comprises an Fc polypeptide comprising a first Fc region variant and a second Fc region variant, each comprising at least one amino acid alteration relative to a parent Fc region, and has a substantially decreased Fc gamma receptor-binding activity and has a maintained or increased C1 q-binding activity when compared to an antigen binding molecule comprising the parent Fc region;
(b) obtaining a gene encoding an antigen-binding molecule in which the first Fc region variant of the antigen-binding molecule selected in (a) is fused to a first antigen-binding moiety which specifically binds to an antigen and the second Fc region variant of the antigen-binding molecule selected in (a) is not fused to any other antigen-binding moiety which specifically binds to the antigen; and
(c) producing an antigen-binding molecule using the gene obtained in (b).

In further embodiments, the first antigen-binding moiety in the antigen-binding molecule encoded by the gene obtained in (b) carries a second antigen-binding moiety fused thereto which specifically binds to an epitope on the antigen that is different from the epitope on the antigen bound by the first antigen-binding moiety. In further embodiments, the first antigen-binding moiety in the antigen-binding molecule encoded by the gene obtained in (b) carries a second antigen-binding moiety fused thereto which specifically binds to the same epitope as the one on the antigen bound by the first antigen-binding moiety. In one non-limiting embodiment, an antigen-binding molecule of the present disclosure can be produced by the production method above.

In one embodiment, the present disclosure provides a method for screening for an antigen-binding molecule, which comprises the steps of:
(a) selecting an antigen-binding molecule that comprises an Fc polypeptide comprising a first Fc region variant and a second Fc region variant, each comprising at least one amino acid alteration relative to a parent Fc region, and has a substantially decreased Fc gamma receptor-binding activity and has a maintained or increased C1 q-binding activity when compared to an antigen binding molecule comprising the parent Fc region;
(b) obtaining a gene encoding an antigen-binding molecule in which the first Fc region variant of the antigen-binding molecule selected in (a) is fused to a first antigen-binding moiety which specifically binds to an antigen and the second Fc region variant of the antigen-binding molecule selected in (a) is not fused to any other antigen-binding moiety which specifically binds to the antigen; and
(c) producing an antigen-binding molecule using the gene obtained in (b).

In further embodiment, the first antigen-binding moiety in the antigen-binding molecule encoded by the gene obtained in (b) carries a second antigen-binding moiety fused thereto which specifically binds to an epitope on the antigen that is different from the epitope on the antigen bound by the first antigen-binding moiety. In further embodiment, the first antigen-binding moiety in the antigen-binding molecule encoded by the gene obtained in (b) carries a second antigen-binding moiety fused thereto which specifically binds to the same epitope as the one on the antigen bound by the first antigen-binding moiety. In one non-limiting embodiment, an antigen-binding molecule of the present disclosure can be produced by the production method above.

Methods for Producing an Antibody with Desired Binding Activity

Methods for producing an antibody with desired binding activity are known to those skilled in the art. Below is an example that describes a method for producing an antibody that binds to HER2.

Anti-HER2 antibodies can be obtained as polyclonal or monoclonal antibodies using known methods. The anti-HER2 antibodies preferably produced are monoclonal antibodies derived from mammals Such mammal-derived monoclonal antibodies include antibodies produced by hybridomas or host cells transformed with an expression vector carrying an antibody gene by genetic engineering techniques.

Monoclonal antibody-producing hybridomas can be produced using known techniques, for example, as described below. Specifically, mammals are immunized by conventional immunization methods using a HER2 protein as a sensitizing antigen. Resulting immune cells are fused with known parental cells by conventional cell fusion methods. Then, hybridomas producing an anti-HER2 antibody can be selected by screening for monoclonal antibody-producing cells using conventional screening methods.

Specifically, monoclonal antibodies are prepared as mentioned below. First, the HER2 gene can be expressed to produce the HER2 protein shown as follows. Those proteins will be used as a sensitizing antigen for antibody preparation. Alternatively, a nucleotide encoding the extracellular domain (ECD) of HER2 can be expressed to produce a HER2 ECD-containing protein. That is, a gene sequence encoding full-length HER2 or HER2 ECD is inserted into a known expression vector, and appropriate host cells are transformed with this vector. The extracellular domain of HER2 could be used. The desired human full-length HER2 or HER2 ECD protein is purified from the host cells or their culture supernatants by known methods. Alternatively, it is possible to use a purified natural HER2 protein as a sensitizing antigen.

The purified full-length HER2 or HER2 ECD protein can be used as a sensitizing antigen for use in immunization of mammals Partial peptides of full-length HER2 or HER2 ECD can also be used as sensitizing antigens. In this case, the partial peptides may also be obtained by chemical synthesis from the human HER2 amino acid sequence. Furthermore, they may also be obtained by incorporating a portion of the HER2 gene into an expression vector and expressing it. Moreover, they may also be obtained by degrading the HER2 protein using proteases, but the region and size of the HER2 peptide used as the partial peptide are not particularly limited to a special embodiment.

For sensitizing antigen, alternatively it is possible to use a fusion protein prepared by fusing a desired partial polypeptide or peptide of the full-length HER2 or HER2 ECD protein with a different polypeptide. For example, antibody Fc fragments and peptide tags are preferably used to produce fusion proteins to be used as sensitizing antigens. Vectors for expression of such fusion proteins can be constructed by fusing in frame genes encoding two or more desired polypeptide fragments and inserting the fusion gene into an expression vector as described above. Methods for producing fusion proteins are described in Molecular Cloning 2nd ed. (Sambrook, J et al., Molecular Cloning 2nd ed., 9.47-9.58 (1989) Cold Spring Harbor Lab. Press). Methods for preparing HER2 to be used as a sensitizing antigen, and immunization methods using HER2 are also generally known.

There is no particular limitation on the mammals to be immunized with the sensitizing antigen. However, it is preferable to select the mammals by considering their compatibility with the parent cells to be used for cell fusion. In general, rodents such as mice, rats, and hamsters, rabbits, and monkeys are preferably used.

The above animals are immunized with a sensitizing antigen by known methods. Generally performed immunization methods include, for example, intraperitoneal or subcutaneous injection of a sensitizing antigen into mammals. Specifically, a sensitizing antigen is appropriately diluted with PBS (Phosphate-Buffered Saline), physiological saline, or the like. If desired, a conventional adjuvant such as Freund's complete adjuvant is mixed with the antigen, and the mixture is emulsified. Then, the sensitizing antigen is administered to a mammal several times at 4- to 21-day intervals. Appropriate carriers may be used in immunization with the sensitizing antigen. In particular, when a low-molecular-weight partial peptide is used as the sensitizing antigen, it is sometimes desirable to couple the sensitizing antigen peptide to a carrier protein such as albumin or keyhole limpet hemocyanin for immunization.

Alternatively, hybridomas producing a desired antibody can be prepared using DNA immunization as mentioned below. DNA immunization is an immunization method that confers immunostimulation by expressing a sensitizing antigen in an animal immunized as a result of administering a vector DNA constructed to allow expression of an antigen protein-encoding gene in the animal. As compared to conventional immunization methods in which a protein antigen is administered to animals to be immunized, DNA immunization is expected to be superior in that:
immunostimulation can be provided while retaining the structure of a membrane protein such as HER2; and
there is no need to purify the antigen for immunization.

In order to prepare a monoclonal antibody of the present invention using DNA immunization, first, a DNA expressing a HER2 protein is administered to an animal to be immunized. The HER2-encoding DNA can be synthesized by known methods such as PCR. The obtained DNA is inserted into an appropriate expression vector, and then this is administered to an animal to be immunized Preferably used expression vectors include, for example, commercially-available expression vectors such as pcDNA3.1. Vectors can be administered to an organism using conventional methods. For example, DNA immunization is performed by using a gene gun to introduce expression vector-coated gold particles into cells in the body of an animal to be immunized Antibodies that recognized HER2 can also be produced by the methods described in WO 2003/104453.

After immunizing a mammal as described above, an increase in the titer of a HER2-binding antibody is confirmed in the serum. Then, immune cells are collected from the mammal, and then subjected to cell fusion. In particular, splenocytes are preferably used as immune cells.

A mammalian myeloma cell is used as a cell to be fused with the above-mentioned immunocyte. The myeloma cells preferably comprise a suitable selection marker for screening. A selection marker confers characteristics to cells for their survival (or death) under a specific culture condition. Hypoxanthine-guanine phosphoribosyltransferase deficiency (hereinafter abbreviated as HGPRT deficiency) and thymidine kinase deficiency (hereinafter abbreviated as TK deficiency) are known as selection markers. Cells with HGPRT or TK deficiency have hypoxanthine-aminopterin-thymidine sensitivity (hereinafter abbreviated as HAT sensitivity). HAT-sensitive cells cannot synthesize DNA in a HAT selection medium, and are thus killed. However, when the cells are fused with normal cells, they can continue DNA synthesis using the salvage pathway of the normal cells, and therefore they can grow even in the HAT selection medium.

HGPRT-deficient and TK-deficient cells can be selected in a medium containing 6-thioguanine, 8-azaguanine (hereinafter abbreviated as 8AG), or 5′-bromodeoxyuridine, respectively. Normal cells are killed because they incorporate these pyrimidine analogs into their DNA. Meanwhile, cells that are deficient in these enzymes can survive in the selection medium, since they cannot incorporate these pyrimidine analogs. In addition, a selection marker referred to as G418 resistance provided by the neomycin-resistant gene confers resistance to 2-deoxystreptamine antibiotics (gentamycin analogs). Various types of myeloma cells that are suitable for cell fusion are known.

For example, myeloma cells including the following cells can be preferably used:

### P3(P3x63Ag8.653) (J. Immunol. (1979) 123 (4), 1548-1550);

### P3x63Ag8U.1 (Current Topics in Microbiology and Immunology (1978) 81, 1-7);

NS-1 (C. Eur. J. Immunol. (1976) 6 (7), 511-519);

### MPC-11 (Cell (1976) 8 (3), 405-415);

### SP2/0 (Nature (1978) 276 (5685), 269-270);

FO (J. Immunol. Methods (1980) 35 (1-2), 1-21);
S194/5.XXO. BU.1 (J. Exp. Med. (1978) 148 (1), 313-323);

### R210 (Nature (1979) 277 (5692), 131-133), etc.

Cell fusions between the immunocytes and myeloma cells are essentially carried out using known methods, for example, a method by Kohler and Milstein et al. (Methods Enzymol. (1981) 73: 3-46).

More specifically, cell fusion can be carried out, for example, in a conventional culture medium in the presence of a cell fusion-promoting agent. The fusion-promoting agents include, for example, polyethylene glycol (PEG) and Sendai virus (HVJ). If required, an auxiliary substance such as dimethyl sulfoxide is also added to improve fusion efficiency.

The ratio of immunocytes to myeloma cells may be determined at one's own discretion, preferably, for example, one myeloma cell for every one to ten immunocytes. Culture media to be used for cell fusions include, for example, media that are suitable for the growth of myeloma cell lines, such as RPMI1640 medium and MEM medium, and other conventional culture medium used for this type of cell culture. In addition, serum supplements such as fetal calf serum (FCS) may be preferably added to the culture medium.

For cell fusion, predetermined amounts of the above immune cells and myeloma cells are mixed well in the above culture medium. Then, a PEG solution (for example, the average molecular weight is about 1,000 to 6,000) prewarmed to about 37 degrees Celsius (C) is added thereto at a concentration of generally 30% to 60% (w/v). This is gently mixed to produce desired fusion cells (hybridomas). Then, an appropriate culture medium mentioned above is gradually added to the cells, and this is repeatedly centrifuged to remove the supernatant. Thus, cell fusion agents and such which are unfavorable to hybridoma growth can be removed.

The hybridomas thus obtained can be selected by culture using a conventional selective medium, for example, HAT medium (a culture medium containing hypoxanthine, aminopterin, and thymidine). Cells other than the desired hybridomas (non-fused cells) can be killed by continuing culture in the above HAT medium for a sufficient period of time. Typically, the period is several days to several weeks. Then, hybridomas producing the desired antibody are screened and singly cloned by conventional limiting dilution methods.

The hybridomas thus obtained can be selected using a selection medium based on the selection marker possessed by the myeloma used for cell fusion. For example, HGPRT- or TK-deficient cells can be selected by culture using the HAT medium (a culture medium containing hypoxanthine, aminopterin, and thymidine). Specifically, when HAT-sensitive myeloma cells are used for cell fusion, cells successfully fused with normal cells can selectively proliferate in the HAT medium. Cells other than the desired hybridomas (non-fused cells) can be killed by continuing culture in the above HAT medium for a sufficient period of time. Specifically, desired hybridomas can be selected by culture for generally several days to several weeks. Then, hybridomas producing the desired antibody are screened and singly cloned by conventional limiting dilution methods.

Desired antibodies can be preferably selected and singly cloned by screening methods based on known antigen/antibody reaction. For example, a HER2-binding monoclonal antibody can bind to HER2 expressed on the cell surface. Such a monoclonal antibody can be screened by fluorescence activated cell sorting (FACS). FACS is a system that assesses the binding of an antibody to cell or cell surface by analyzing cells contacted with a fluorescent antibody using laser beam, and measuring the fluorescence emitted from individual cells.

To screen for hybridomas that produce a monoclonal antibody of the present invention by FACS, HER2-expressing cells are first prepared. Cells preferably used for screening are mammalian cells in which HER2 is forcedly expressed. As control, the activity of an antibody to bind to cell-surface HER2 can be selectively detected using non-transformed mammalian cells as host cells. Specifically, hybridomas producing an anti-HER2 monoclonal antibody can be isolated by selecting hybridomas that produce an antibody which binds to cells forced to express HER2, but not to host cells.

Alternatively, the activity of an antibody to bind to immobilized HER2-expressing cells can be assessed based on the principle of ELISA. For example, HER2-expressing cells are immobilized to the wells of an ELISA plate. Culture supernatants of hybridomas are contacted with the immobilized cells in the wells, and antibodies that bind to the immobilized cells are detected. When the monoclonal antibodies are derived from mouse, antibodies bound to the cells can be detected using an anti-mouse immunoglobulin antibody. Hybridomas producing a desired antibody having the antigen-binding ability are selected by the above screening, and they can be cloned by a limiting dilution method or the like.

Monoclonal antibody-producing hybridomas thus prepared can be passaged in a conventional culture medium, and stored in liquid nitrogen for a long period.

The above hybridomas are cultured by a conventional method, and desired monoclonal antibodies can be prepared from the culture supernatants. Alternatively, the hybridomas are administered to and grown in compatible mammals, and monoclonal antibodies are prepared from the ascites. The former method is suitable for preparing antibodies with high purity.

Antibodies encoded by antibody genes that are cloned from antibody-producing cells such as the above hybridomas can also be preferably used. A cloned antibody gene is inserted into an appropriate vector, and this is introduced into a host to express the antibody encoded by the gene. Methods for isolating antibody genes, inserting the genes into vectors, and transforming host cells have already been established, for example, by Vandamme et al. (Eur. J. Biochem. (1990) 192(3), 767-775). Methods for producing recombinant antibodies are also known as described below.

Preferably, the present invention provides nucleic acids that encode an antigen-binding molecule of the present invention. The present invention also provides a vector into which the nucleic acid encoding the antigen-binding molecule is introduced, i.e., a vector comprising the nucleic acid. Furthermore, the present invention provides a cell comprising the nucleic acid or the vector. The present invention also provides a method for producing the antigen-binding molecule by culturing the cell. The present invention further provides antigen-binding molecules produced by the method.

For example, a cDNA encoding the variable region (V region) of an anti-HER2 antibody is prepared from hybridoma cells expressing the anti-HER2 antibody. For this purpose, total RNA is first extracted from hybridomas. Methods used for extracting mRNAs from cells include, for example:
the guanidine ultracentrifugation method (Biochemistry (1979) 18(24), 5294-5299), and
the AGPC method (Anal. Biochem. (1987) 162(1), 156-159)

Extracted mRNAs can be purified using the mRNA Purification Kit (GE Healthcare Bioscience) or such. Alternatively, kits for extracting total mRNA directly from cells, such as the QuickPrep mRNA Purification Kit (GE Healthcare Bioscience), are also commercially available. mRNAs can be prepared from hybridomas using such kits. cDNAs encoding the antibody V region can be synthesized from the prepared mRNAs using a reverse transcriptase. cDNAs can be synthesized using the AMV Reverse Transcriptase First-strand cDNA Synthesis Kit (Seikagaku Co.) or such. Furthermore, the SMART RACE cDNA amplification kit (Clontech) and the PCR-based 5′-RACE method (Proc. Natl. Acad. Sci. USA (1988) 85(23), 8998-9002; Nucleic Acids Res. (1989) 17(8), 2919-2932) can be appropriately used to synthesize and amplify cDNAs. In such a cDNA synthesis process, appropriate restriction enzyme sites described below may be introduced into both ends of a cDNA.

The cDNA fragment of interest is purified from the resulting PCR product, and then this is ligated to a vector DNA. A recombinant vector is thus constructed, and introduced into E. coli or such. After colony selection, the desired recombinant vector can be prepared from the colony-forming E. coli. Then, whether the recombinant vector has the cDNA nucleotide sequence of interest is tested by a known method such as the dideoxy nucleotide chain termination method.

The 5′-RACE method which uses primers to amplify the variable region gene is conveniently used for isolating the gene encoding the variable region. First, a 5′-RACE cDNA library is constructed by cDNA synthesis using RNAs extracted from hybridoma cells as a template. A commercially available kit such as the SMART RACE cDNA amplification kit is appropriately used to synthesize the 5′-RACE cDNA library.

The antibody gene is amplified by PCR using the prepared 5′-RACE cDNA library as a template. Primers for amplifying the mouse antibody gene can be designed based on known antibody gene sequences. The nucleotide sequences of the primers vary depending on the immunoglobulin subclass. Therefore, it is preferable that the subclass is determined in advance using a commercially available kit such as the Iso Strip mouse monoclonal antibody isotyping kit (Roche Diagnostics).

Specifically, for example, primers that allow amplification of genes encoding gammal, gamma2a, gamma2b, and gamma3 heavy chains and kappa and lambda light chains are used to isolate mouse IgG-encoding genes. In general, a primer that anneals to a constant region site close to the variable region is used as a 3′-side primer to amplify an IgG variable region gene. Meanwhile, a primer attached to a 5′ RACE cDNA library construction kit is used as a 5′-side primer.

PCR products thus amplified are used to reshape immunoglobulins composed of a combination of heavy and light chains. A desired antibody can be selected using the HER2-binding activity of a reshaped immunoglobulin as an indicator. For example, when the objective is to isolate an antibody against HER2, it is more preferred that the binding of the antibody to HER2 is specific. A HER2-binding antibody can be screened, for example, by the following steps:
(1) contacting a HER2-expressing cell with an antibody comprising the V region encoded by a cDNA isolated from a hybridoma;
(2) detecting the binding of the antibody to the HER2-expressing cell; and
(3) selecting an antibody that binds to the HER2-expressing cell.

Methods for detecting the binding of an antibody to HER2-expressing cells are known. Specifically, the binding of an antibody to HER2-expressing cells can be detected by the above-described techniques such as FACS Immobilized samples of HER2-expressing cells are appropriately used to assess the binding activity of an antibody.

Preferred antibody screening methods that use the binding activity as an indicator also include panning methods using phage vectors. Screening methods using phage vectors are advantageous when the antibody genes are isolated from heavy-chain and light-chain subclass libraries from a polyclonal antibody-expressing cell population. Genes encoding the heavy-chain and light-chain variable regions can be linked by an appropriate linker sequence to form a single-chain Fv (scFv). Phages presenting scFv on their surface can be produced by inserting a gene encoding scFv into a phage vector. The phages are contacted with an antigen of interest. Then, a DNA encoding scFv having the binding activity of interest can be isolated by collecting phages bound to the antigen. This process can be repeated as necessary to enrich scFv having a desired binding activity.

After isolation of the cDNA encoding the V region of the anti-HER2 antibody of interest, the cDNA is digested with restriction enzymes that recognize the restriction sites introduced into both ends of the cDNA. Preferred restriction enzymes recognize and cleave a nucleotide sequence that occurs in the nucleotide sequence of the antibody gene at a low frequency. Furthermore, a restriction site for an enzyme that produces a sticky end is preferably introduced into a vector to insert a single-copy digested fragment in the correct orientation. The cDNA encoding the V region of the anti-HER2 antibody is digested as described above, and this is inserted into an appropriate expression vector to construct an antibody expression vector. In this case, if a gene encoding the antibody constant region (C region) and a gene encoding the above V region are fused in-frame, a chimeric antibody is obtained. Herein, “chimeric antibody” means that the origin of the constant region is different from that of the variable region. Thus, in addition to mouse/human heterochimeric antibodies, human/human allochimeric antibodies are included in the chimeric antibodies of the present invention. A chimeric antibody expression vector can be constructed by inserting the above V region gene into an expression vector that already has the constant region. Specifically, for example, a recognition sequence for a restriction enzyme that excises the above V region gene can be appropriately placed on the 5′ side of an expression vector carrying a DNA encoding a desired antibody constant region (C region). A chimeric antibody expression vector is constructed by fusing in frame the two genes digested with the same combination of restriction enzymes.

To produce an anti-HER2 monoclonal antibody, antibody genes are inserted into an expression vector so that the genes are expressed under the control of an expression regulatory region. The expression regulatory region for antibody expression includes, for example, enhancers and promoters. Furthermore, an appropriate signal sequence may be attached to the amino terminus so that the expressed antibody is secreted to the outside of cells. The expressed polypeptide is cleaved typically at the carboxyl terminus of the signal sequence, and the resulting polypeptide is secreted to the outside of cells as a mature polypeptide. Then, appropriate host cells are transformed with the expression vector, and recombinant cells expressing the anti-HER2 antibody-encoding DNA are obtained.

DNAs encoding the antibody heavy chain (H chain) and light chain (L chain) are separately inserted into different expression vectors to express the antibody gene. An antibody molecule having the H and L chains can be expressed by co-transfecting the same host cell with vectors into which the H-chain and L-chain genes are respectively inserted. Alternatively, host cells can be transformed with a single expression vector into which DNAs encoding the H and L chains are inserted (see WO 94/11523).

There are various known host cell/expression vector combinations for antibody preparation by introducing isolated antibody genes into appropriate hosts. All of these expression systems are applicable to isolation of domains including antibody variable regions of the present invention. Appropriate eukaryotic cells used as host cells include animal cells, plant cells, and fungal cells. Specifically, the animal cells include, for example, the following cells.
(1) mammalian cells: CHO, COS, myeloma, baby hamster kidney (BHK), HeLa, Vero, or such;
(2) amphibian cells: Xenopus oocytes, or such; and
(3) insect cells: sf9, sf21, Tn5, or such.

In addition, as a plant cell, an antibody gene expression system using cells derived from the Nicotiana genus such as Nicotiana tabacum is known. Callus cultured cells can be appropriately used to transform plant cells.

Furthermore, the following cells can be used as fungal cells: yeasts: the Saccharomyces genus such as Saccharomyces cerevisiae, and the Pichia genus such as Pichia pastoris; and filamentous fungi: the Aspergillus genus such as Aspergillus niger.

Furthermore, antibody gene expression systems that utilize prokaryotic cells are also known. For example, when using bacterial cells, E. coli cells, Bacillus subtilis cells, and such can suitably be utilized in the present invention. Expression vectors carrying the antibody genes of interest are introduced into these cells by transfection. The transfected cells are cultured in vitro, and the desired antibody can be prepared from the culture of transformed cells.

In addition to the above-described host cells, transgenic animals can also be used to produce a recombinant antibody. That is, the antibody can be obtained from an animal into which the gene encoding the antibody of interest is introduced. For example, the antibody gene can be constructed as a fusion gene by inserting in frame into a gene that encodes a protein produced specifically in milk. Goat beta-casein or such can be used, for example, as the protein secreted in milk. DNA fragments containing the fused gene inserted with the antibody gene is injected into a goat embryo, and then this embryo is introduced into a female goat. Desired antibodies can be obtained as a protein fused with the milk protein from milk produced by the transgenic goat born from the embryo-recipient goat (or progeny thereof). In addition, to increase the volume of milk containing the desired antibody produced by the transgenic goat, hormones can be administered to the transgenic goat as necessary (Ebert, K. M. et al., Bio/Technology (1994) 12 (7), 699-702).

### Methods for Producing a Humanized Antibody

When an antigen-binding molecule described herein is administered to human, a domain derived from a genetically recombinant antibody that has been artificially modified to reduce the heterologous antigenicity against human and such, can be appropriately used as the domain of the antigen-binding molecule including an antibody variable region. Such genetically recombinant antibodies include, for example, humanized antibodies. These modified antibodies are appropriately produced by known methods. Furthermore, generally, the binding specificity of a certain antibody can be introduced into another antibody by CDR grafting.

Specifically, humanized antibodies prepared by grafting the CDR of a non-human animal antibody such as a mouse antibody to a human antibody and such are known. Common genetic engineering techniques for obtaining humanized antibodies are also known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the FR adjacent to the mouse CDR to be grafted.

Nucleotide sequences to be ligated are designed so that they will be connected to each other in frame. Human FRs are individually synthesized using the respective primers. As a result, products in which the mouse CDR-encoding DNA is attached to the individual FR-encoding DNAs are obtained. Nucleotide sequences encoding the mouse CDR of each product are designed so that they overlap with each other. Then, complementary strand synthesis reaction is conducted to anneal the overlapping CDR regions of the products synthesized using a human antibody gene as template. Human FRs are ligated via the mouse CDR sequences by this reaction.

The full-length V region gene, in which three CDRs and four FRs are ultimately ligated, is amplified using primers that anneal to its 5′- or 3′-end, which are added with suitable restriction enzyme recognition sequences. An expression vector for humanized antibody can be produced by inserting the DNA obtained as described above and a DNA that encodes a human antibody C region into an expression vector so that they will ligate in frame. After the recombinant vector is transfected into a host to establish recombinant cells, the recombinant cells are cultured, and the DNA encoding the humanized antibody is expressed to produce the humanized antibody in the cell culture (see, European Patent Publication No. EP 239400 and International Patent Publication No. WO 1996/002576).

By qualitatively or quantitatively measuring and evaluating the antigen-binding activity of the humanized antibody produced as described above, one can suitably select human antibody FRs that allow CDRs to form a favorable antigen-binding site when ligated through the CDRs Amino acid residues in FRs may be substituted as necessary, so that the CDRs of a reshaped human antibody form an appropriate antigen-binding site. For example, amino acid sequence mutations can be introduced into FRs by applying the PCR method used for grafting a mouse CDR into a human FR. More specifically, partial nucleotide sequence mutations can be introduced into primers that anneal to the FR. Nucleotide sequence mutations are introduced into the FRs synthesized by using such primers. Mutant FR sequences having the desired characteristics can be selected by measuring and evaluating the activity of the amino acid-substituted mutant antibody to bind to the antigen by the above-mentioned method (Sato, K. et al., Cancer Res. (1993) 53: 851-856)

### Methods for Producing a Human Antibody

Alternatively, desired human antibodies can be obtained by immunizing transgenic animals having the entire repertoire of human antibody genes (see WO 1993/012227; WO 1992/003918; WO 1994/002602; WO 1994/025585; WO 1996/034096; WO 1996/033735) by DNA immunization.

Furthermore, techniques for preparing human antibodies by panning using human antibody libraries are also known. For example, the V region of a human antibody is expressed as a single-chain antibody (scFv) on phage surface by the phage display method. Phages expressing a scFv that binds to the antigen can be selected. The DNA sequence encoding the human antibody V region that binds to the antigen can be determined by analyzing the genes of selected phages. The DNA sequence of the scFv that binds to the antigen is determined. An expression vector is prepared by fusing the V region sequence in frame with the C region sequence of a desired human antibody, and inserting this into an appropriate expression vector. The expression vector is introduced into cells appropriate for expression such as those described above. The human antibody can be produced by expressing the human antibody-encoding gene in the cells. These methods are already known (see WO 1992/001047; WO 1992/020791; WO 1993/006213; WO 1993/011236; WO 1993/019172; WO 1995/001438; WO 1995/015388).

### Epitope

“Epitope” means an antigenic determinant in an antigen, and refers to an antigen site to which the antigen-binding domain or moiety of an antigen-binding molecule or antibody disclosed herein binds. Thus, for example, the epitope can be defined according to its structure. Alternatively, the epitope may be defined according to the antigen-binding activity of an antigen-binding molecule or antibody that recognizes the epitope. When the antigen is a peptide or polypeptide, the epitope can be specified by the amino acid residues forming the epitope. Alternatively, when the epitope is a sugar chain, the epitope can be specified by its specific sugar chain structure.

A linear epitope is an epitope that contains an epitope whose primary amino acid sequence is recognized. Such a linear epitope typically contains at least three and most commonly at least five, for example, about 8 to 10 or 6 to 20 amino acids in its specific sequence.

In contrast to the linear epitope, “conformational epitope” is an epitope in which the primary amino acid sequence containing the epitope is not the only determinant of the recognized epitope (for example, the primary amino acid sequence of a conformational epitope is not necessarily recognized by an epitope-defining antibody). Conformational epitopes may contain a greater number of amino acids compared to linear epitopes. A conformational epitope-recognizing antigen-binding domain recognizes the three-dimensional structure of a peptide or protein. For example, when a protein molecule folds and forms a three-dimensional structure, amino acids and/or polypeptide main chains that form a conformational epitope become aligned, and the epitope is made recognizable by the antigen-binding domain. Methods for determining epitope conformations include, for example, X ray crystallography, two-dimensional nuclear magnetic resonance, site-specific spin labeling, and electron paramagnetic resonance, but are not limited thereto. See, for example, Epitope Mapping Protocols in Methods in Molecular Biology (1996), Vol. 66, Morris (ed.).

Examples of a method for assessing the epitope binding by a test antigen-binding molecule or antibody containing an anti-HER2 antigen-binding domain are described below. According to the examples below, methods for assessing the epitope binding by a test antigen-binding molecule or antibody containing an antigen-binding domain for an antigen other than HER2, can also be appropriately conducted.

For example, whether a test antigen-binding molecule or antibody containing an anti-HER2 antigen-binding domain or moiety recognizes a linear epitope in the HER2 molecule can be confirmed for example as mentioned below. A linear peptide comprising an amino acid sequence forming the extracellular domain of HER2 is synthesized for the above purpose. The peptide can be synthesized chemically, or obtained by genetic engineering techniques using a region encoding the amino acid sequence corresponding to the extracellular domain in a HER2 cDNA. Then, a test antigen-binding molecule or antibody containing an anti-HER2 antigen-binding domain or moiety is assessed for its binding activity towards a linear peptide comprising the amino acid sequence forming the extracellular domain. For example, an immobilized linear peptide can be used as an antigen by ELISA to evaluate the binding activity of the polypeptide complex towards the peptide. Alternatively, the binding activity towards a linear peptide can be assessed based on the level that the linear peptide inhibits the binding of the antigen-binding molecule or antibody to HER2-expressing cells. These tests can demonstrate the binding activity of the antigen-binding molecule or antibody towards the linear peptide.

Whether a test antigen-binding molecule or antibody containing an anti-HER2 antigen-binding domain recognizes a conformational epitope can be assessed as follows. HER2-expressing cells are prepared for the above purpose. A test antigen-binding molecule or antibody containing an anti-HER2 antigen-binding domain or moiety can be determined to recognize a conformational epitope when it strongly binds to HER2-expressing cells upon contact, but does not substantially bind to an immobilized linear peptide comprising an amino acid sequence forming the extracellular domain of HER2. Herein, “not substantially bind” means that the binding activity is 80% or less, generally 50% or less, preferably 30% or less, and particularly preferably 15% or less compared to the binding activity towards cells expressing a HER2.

Methods for assaying the binding activity of a test antigen-binding molecule or antibody containing an anti-HER2 antigen-binding domain towards HER2-expressing cells include, for example, the methods described in Antibodies: A Laboratory Manual (Ed Harlow, David Lane, Cold Spring Harbor Laboratory (1988) 359-420). Specifically, the assessment can be performed based on the principle of ELISA or fluorescence activated cell sorting (FACS) using HER2-expressing cells as antigen.

In the ELISA format, the binding activity of a test antigen-binding molecule or antibody containing an anti-HER2 antigen-binding domain towards HER2-expressing cells can be assessed quantitatively by comparing the levels of signal generated by enzymatic reaction. Specifically, a test polypeptide complex is added to an ELISA plate onto which HER2-expressing cells are immobilized. Then, the test antigen-binding molecule or antibody bound to the cells is detected using an enzyme-labeled antibody that recognizes the test antigen-binding molecule or antibody. Alternatively, when FACS is used, a dilution series of a test antigen-binding molecule or antibody is prepared, and the antibody binding titer for HER2-expressing cells can be determined to compare the binding activity of the test antigen-binding molecule or antibody towards HER2-expressing cells.

The binding of a test antigen-binding molecule or antibody towards an antigen expressed on the cells or surface of cells suspended in buffer or the like can be detected using a flow cytometer. Known flow cytometers include, for example, the following devices:
FACSCanto™ II
FACSAria™
FACSArray™
FACSVantage™ SE
FACSCalibur™ (all are trade names of BD Biosciences)
EPICS ALTRA HyPerSort
Cytomics FC 500
EPICS XL-MCL ADC EPICS XL ADC
Cell Lab Quanta/Cell Lab Quanta SC (all are trade names of Beckman Coulter)

Preferable methods for assaying the binding activity of a test antigen-binding molecule or antibody containing an anti-HER2 antigen-binding domain towards an antigen include, for example, the following method. First, HER2-expressing cells are reacted with a test antigen-binding molecule or antibody, and then this is stained with an FITC-labeled secondary antibody that recognizes the antigen-binding molecule or antibody. The test antigen-binding molecule or antibody is appropriately diluted with a suitable buffer to prepare the antigen-binding molecule or antibody at a desired concentration. For example, the antigen-binding molecule or antibody can be used at a concentration within the range of 10 micro g/ml to 10 ng/ml. Then, the fluorescence intensity and cell count are determined using FACSCalibur (BD). The fluorescence intensity obtained by analysis using the CELL QUEST Software (BD), i.e., the Geometric Mean value, reflects the quantity of antibody bound to cells. That is, the binding activity of a test antigen-binding molecule or antibody, which is represented by the quantity of the test antigen-binding molecule or antibody bound, can be determined by measuring the Geometric Mean (Geo-mean) value.

Whether a test antigen-binding molecule or antibody containing an anti-HER2 antigen-binding domain shares a common epitope with another antigen-binding molecule or antibody can be assessed based on the competition between the two antigen-binding molecules or antibodies for the same epitope. The competition between the antigen-binding molecules or antibodies can be detected by cross-blocking assay or the like. For example, the competitive ELISA assay is a preferred cross-blocking assay.

Specifically, in cross-blocking assay, the HER2 protein immobilized to the wells of a microtiter plate is pre-incubated in the presence or absence of a candidate competitor antigen-binding molecule or antibody, and then a test antigen-binding molecule or antibody is added thereto. The quantity of test antigen-binding molecule or antibody bound to the HER2 protein in the wells is indirectly correlated with the binding ability of a candidate competitor antigen-binding molecule or antibody that competes for the binding to the same epitope. That is, the greater the affinity of the competitor antigen-binding molecule or antibody for the same epitope, the lower the binding activity of the test antigen-binding molecule or antibody towards the HER2 protein-coated wells.

The quantity of the test antigen-binding molecule or antibody bound to the wells via the HER2 protein can be readily determined by labeling the antigen-binding molecule or antibody in advance. For example, a biotin-labeled antigen-binding molecule or antibody is measured using an avidin/peroxidase conjugate and appropriate substrate. In particular, cross-blocking assay that uses enzyme labels such as peroxidase is called “competitive ELISA assay”. The antigen-binding molecule or antibody can also be labeled with other labeling substances that enable detection or measurement. Specifically, radiolabels, fluorescent labels, and such are known.

When the candidate competitor antigen-binding molecule or antibody can block the binding by a test antigen-binding molecule or antibody containing an anti-HER2 antigen-binding domain by at least 20%, preferably at least 20 to 50%, and more preferably at least 50% compared to the binding activity in a control experiment conducted in the absence of the competitor antigen-binding molecule or antibody, the test antigen-binding molecule or antibody is determined to substantially bind to the same epitope bound by the competitor antigen-binding molecule or antibody, or compete for the binding to the same epitope.

When the structure of an epitope bound by a test antigen-binding molecule or antibody containing an anti-HER2 antigen-binding domain has already been identified, whether the test and control antigen-binding molecules or antibodies share a common epitope can be assessed by comparing the binding activities of the two antigen-binding molecules or antibodies towards a peptide prepared by introducing amino acid mutations into the peptide forming the epitope.

To measure the above binding activities, for example, the binding activities of test and control antigen-binding molecules or antibodies towards a linear peptide into which a mutation is introduced are compared in the above ELISA format. Besides the ELISA methods, the binding activity towards the mutant peptide bound to a column can be determined by flowing test and control antigen-binding molecules or antibodies in the column, and then quantifying the antigen-binding molecule or antibody eluted in the elution solution. Methods for adsorbing a mutant peptide to a column, for example, in the form of a GST fusion peptide, are known.

Alternatively, when the identified epitope is a conformational epitope, whether test and control antigen-binding molecules or antibodies share a common epitope can be assessed by the following method. First, HER2-expressing cells and cells expressing a HER2 with a mutation introduced into the epitope are prepared. The test and control antigen-binding molecules or antibodies are added to a cell suspension prepared by suspending these cells in an appropriate buffer such as PBS. Then, the cell suspensions are appropriately washed with a buffer, and an FITC-labeled antibody that recognizes the test and control antigen-binding molecules or antibodies is added thereto. The fluorescence intensity and number of cells stained with the labeled antibody are determined using FACSCalibur™ (BD). The test and control antigen-binding molecules or antibodies are appropriately diluted using a suitable buffer, and used at desired concentrations. For example, they may be used at a concentration within the range of 10 micro g/ml to 10 ng/ml. The fluorescence intensity determined by analysis using the CELLQUEST™ Software (BD), i.e., the Geometric Mean value, reflects the quantity of labeled antibody bound to cells. That is, the binding activities of the test and control antigen-binding molecules or antibodies, which are represented by the quantity of labeled antibody bound, can be determined by measuring the Geometric Mean value.

In the above method, whether an antigen-binding molecule or antibody does “not substantially bind to cells expressing mutant HER2” can be assessed, for example, by the following method. First, the test and control antigen-binding molecules or antibodies bound to cells expressing mutant HER2 are stained with a labeled antibody. Then, the fluorescence intensity of the cells is determined. When FACSCalibur™ is used for fluorescence detection by flow cytometry, the determined fluorescence intensity can be analyzed using the CELLQUEST™ Software. From the Geometric Mean values in the presence and absence of the antigen-binding molecule or antibody, the comparison value (delta Geo-Mean) can be calculated according to the following formula to determine the ratio of increase in fluorescence intensity as a result of the binding by the antigen-binding molecule or antibody.

delta Geo-Mean=Geo-Mean(in the presence of the antigen-binding molecule or antibody)/Geo-Mean(in the absence of the antigen-binding molecule or antibody)

The Geometric Mean comparison value (delta Geo-Mean value for the mutant HER2 molecule) determined by the above analysis, which reflects the quantity of a test antigen-binding molecule or antibody bound to cells expressing mutant HER2, is compared to the delta Geo-Mean comparison value that reflects the quantity of the test antigen-binding molecule or antibody bound to HER2-expressing cells. In this case, the concentrations of the test antigen-binding molecule or antibody used to determine the delta Geo-Mean comparison values for HER2-expressing cells and cells expressing mutant HER2 are particularly preferably adjusted to be equal or substantially equal. An antigen-binding molecule or antibody that has been confirmed to recognize an epitope in HER2 is used as a control antigen-binding molecule or antibody.

If the delta Geo-Mean comparison value of a test antigen-binding molecule or antibody for cells expressing mutant HER2 is smaller than the delta Geo-Mean comparison value of the test antigen-binding molecule or antibody for HER2-expressing cells by at least 80%, preferably 50%, more preferably 30%, and particularly preferably 15%, then the test antigen-binding molecule or antibody “does not substantially bind to cells expressing mutant HER2”. The formula for determining the Geo-Mean (Geometric Mean) value is described in the CELL QUEST Software User's Guide (BD biosciences). When the comparison shows that the comparison values are substantially equivalent, the epitope for the test and control antigen-binding molecules or antibodies can be determined to be the same.

### Production and Purification of Antigen-Binding Molecules

In some embodiments, the antigen-binding molecules of the present disclosure are isolated antigen-binding molecules.

In one embodiment, the antigen-binding molecules described herein comprise at least one or more antigen-binding moieties (e.g. the “first antigen-binding moiety” and the “second antigen-binding moiety”), fused to one of the two subunits of the Fc polypeptide (the first Fc region variant), thus the two subunits of the Fc domain are typically comprised in two non-identical polypeptide chains. Recombinant co-expression of these polypeptide chains and subsequent dimerization leads to several possible combinations of the two polypeptide chains. To improve the yield and purity of antigen-binding molecules in recombinant production, it will thus be advantageous to introduce in the Fc domain of the antigen-binding molecule a modification promoting the association of the desired polypeptide chains.

Accordingly, in particular embodiments, the Fc domain of the antigen-binding molecule described herein comprises a modification promoting the association of the first and the second subunit of the Fc domain. The site of most extensive protein-protein interaction between the two subunits of a human IgG Fc domain is in the CH3 domain of the Fc domain. Thus, in one embodiment, said modification is in the CH3 domain of the Fc domain.

In a specific embodiment, said modification is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the antigen-binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In other embodiments, other techniques for promoting the association among H chains and between L and H chains having the desired combinations can be applied to the antigen-binding molecules of the present disclosure.

Furthermore, an Fc region whose Fc region C-terminal heterogeneity has been improved can be appropriately used as an Fc region of the present disclosure. More specifically, the present disclosure provides Fc regions produced by deleting glycine at position 446 and lysine at position 447 as specified by EU numbering from the amino acid sequences of two polypeptides constituting an Fc region derived from IgG1, IgG2, IgG3, or IgG4.

Antigen-binding molecules prepared as described herein may be purified by art-known techniques such as high-performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the antigen-binding molecule binds. For example, for affinity chromatography purification of antigen-binding molecules of the invention, a matrix with protein A or protein G may be used. Sequential Protein A or G affinity chromatography and size exclusion chromatography can be used to isolate an antigen-binding molecule. The purity of the antigen-binding molecule can be determined by any of a variety of well-known analytical methods including gel electrophoresis, high pressure liquid chromatography, and the like.

### Pharmaceutical Composition

In one aspect, the present disclosure provides a pharmaceutical composition comprising the antigen-binding molecule of the disclosure.

Any of the antigen-binding molecules comprising a variant Fc region or a variant Fc polypeptide which comprises a first Fc region variant and a second Fc region variant provided herein may be used in therapeutic methods.

In one aspect, an antigen-binding molecule comprising a variant Fc region or polypeptide for use as a medicament is provided. In certain embodiments, an antigen-binding molecule comprising a variant Fc region or polypeptide for use in a method of treatment is provided. In some aspects, the antigen-binding molecule is a one-armed antibody. In some aspects, the antigen-binding molecule is an Fc fusion protein. In certain embodiments, the invention provides an antigen-binding molecule comprising a variant Fc region or polypeptide for use in a method of treating an individual having a disorder comprising administering to the individual an effective amount of the antigen-binding molecule comprising the variant Fc region or polypeptide. In one such embodiment, the method further comprises administering to the individual an effective amount of at least one additional therapeutic agent. In one embodiment, the disorder is a viral infection. In one embodiment, the “individual” is a human.

In a further aspect, the present disclosure provides for the use of an antigen-binding molecule comprising a variant Fc region or polypeptide in the manufacture or preparation of a medicament. In one embodiment, the medicament is for treatment of a disorder. In some aspects, the antigen-binding molecule is a one-armed antibody. In some aspects, the antigen-binding molecule is an Fc fusion protein. In a further embodiment, the medicament is for use in a method of treating a disorder comprising administering to an individual having the disorder to be treated an effective amount of the medicament. In one such embodiment, the method further comprises administering to the individual an effective amount of at least one additional therapeutic agent. In one embodiment, the disorder is a viral infection. In one embodiment, the “individual” is a human.

In a further aspect, the present disclosure provides a method for treating a disorder. In one embodiment, the method comprises administering to an individual having such a disorder an effective amount of an antigen-binding molecule comprising a variant Fc region or polypeptide. In some aspects, the antigen-binding molecule is a one-armed antibody. In some aspects, the antigen-binding molecule is an Fc fusion protein. In one such embodiment, the method further comprises administering to the individual an effective amount of at least one additional therapeutic agent. In one embodiment, the disorder is a viral infection. In one embodiment, the “individual” is a human.

In a further aspect, the invention provides pharmaceutical formulations comprising an antigen-binding molecule comprising a variant Fc region or polypeptide provided herein. In one embodiment, a pharmaceutical formulation is for use in a therapeutic method such as any of the therapeutic methods described herein. In one embodiment, a pharmaceutical formulation comprises an antigen-binding molecule comprising a variant Fc region or polypeptide provided herein and a pharmaceutically acceptable carrier. In another embodiment, a pharmaceutical formulation comprises an antigen-binding molecule comprising a variant Fc region or polypeptide provided herein and at least one additional therapeutic agent. In some aspects, the antigen-binding molecule is a one-armed antibody. In some aspects, the antigen-binding molecule is an Fc fusion protein.

In a further aspect, the pharmaceutical formulation is for treatment of a disorder. In one embodiment, the pharmaceutical formulation is administered to an individual having a disorder. In one embodiment, the disorder is a viral infection. In one embodiment, the “individual” is a human.

In one aspect, the pharmaceutical formulation or pharmaceutical composition herein may comprise two or more of the antigen-binding molecules of the present disclosure. For example, the pharmaceutical formulation comprises a first antigen-binding molecule comprising a variant Fc region or polypeptide provided herein and a second antigen-binding molecule comprising a variant Fc region or polypeptide provided herein. The first and the second antigen-binding molecules are different from each other. The first and the second antigen-binding molecules may bind to different epitopes on the same antigen or to different epitopes on different antigens. For example, the first antigen-binding molecule may be a “one-armed” antigen binding molecule comprising (i) an antigen-binding moiety that specifically binds to an epitope on an antigen and (ii) an Fc polypeptide that comprises a first and a second Fc region variant provided herein, the first Fc region variant being fused to the antigen-binding moiety while the second Fc region variant being not fused to any antigen-binding moiety, and the second antigen-binding molecule may be a “one-armed” antigen binding molecule comprising (i) an antigen-binding moiety that specifically binds to an epitope on an antigen and (ii) an Fc polypeptide that comprises a first and a second Fc region variant provided herein, the first Fc region variant being fused to the antigen-binding moiety while the second Fc region variant being not fused to any antigen-binding moiety. The first and the second antigen-binding molecules in this example are “different” from each other, for example, in that they bind to different epitopes on the same antigen, but they may be different in other aspects such as number of antigen-binding moieties contained in the molecule, etc.

In one aspect, the invention provides a therapeutic agent or a pharmaceutical composition comprising a first antigen-binding molecule comprising a variant Fc region or polypeptide provided herein as an active ingredient, for combined administration with a second antigen-binding molecule comprising a variant Fc region or polypeptide provided herein. The pharmaceutical composition is for treating or preventing a disease, more particularly, a viral infectious disease. The pharmaceutical composition comprising the first antigen-binding molecule is administered to a subject simultaneously, separately, or sequentially with the second antigen-binding molecule. Stating differently, the pharmaceutical composition comprising the first antigen-binding molecule and the pharmaceutical composition comprising the second antigen-binding molecule may be administered in parallel (i.e., concurrently) or sequentially (i.e., at different times). In some embodiments where the first antigen-binding molecule and the second antigen-binding molecule are administered sequentially (i.e., at different times), the time interval between the administrations is not particularly limited and it may be determined suitably in consideration of various factors including administration routs and dosage forms. For example, the time interval may be between 0 to 168 hours, 0 to 72 hours, 0 to 24 hours, or 0 to 12 hours but it is not limited to these examples. In some embodiments, the first antigen-binding molecule and the second antigen-binding molecule are administered simultaneously. In some other embodiments, the first antigen-binding molecule and the second antigen-binding molecule are administered at an interval. The first antigen-binding molecule and the second antigen-binding molecule may be formulated into the same dosage form, or different dosage forms. For example, the first and the second antigen-binding molecules may be formulated as different forms selected from parenteral preparations, injections, infusions, intravenous infusions, etc. Alternatively, the first and the second antigen-binding molecules may both be formulated as one of parenteral preparation, injection, infusion, intravenous infusion, etc. The present disclosure provides a first antigen-binding molecule comprising a variant Fc region or polypeptide provided herein, for use in treatment or prevention of a viral infectious disease in combination with a second antigen-binding molecule comprising a variant Fc region or polypeptide provided herein. The present disclosure further provides a method for treating or preventing a viral infectious disease comprising administering to a subject a first antigen-binding molecule comprising a variant Fc region or polypeptide provided herein and administering to the subject a second antigen-binding molecule comprising a variant Fc region or polypeptide provided herein. The present disclosure further provides use of a first antigen-binding molecule comprising a variant Fc region or polypeptide provided herein in the manufacture of a medicament for treating or preventing a viral infectious disease, the medicament being for combined administration with the second antigen-binding molecule comprising a variant Fc region or polypeptide provided herein. The first and the second antigen-binding molecules are different from each other. The first and the second antigen-binding molecule may bind to different epitopes on the same antigen or to different epitopes on different antigens. In one embodiment, the first antigen-binding molecule and the second antigen-binding molecule are for use in a therapeutic method such as any of the therapeutic methods described herein. In some embodiments, the first and the second antigen-binding molecules are one-armed antibodies. In some embodiments, the first and the second antigen-binding molecules are Fc fusion proteins. In one embodiment, the first and the second antigen-binding molecules are “one-armed” antigen-binding molecules, binding to different epitopes on the same antigen.

In one aspect, the present disclosure provides a pharmaceutical composition comprising a combination of a first antigen-binding molecule comprising a variant Fc region or polypeptide provided herein and a second antigen-binding molecule comprising a variant Fc region or polypeptide provided herein. The pharmaceutical composition is for treating or preventing a disease, more particularly a viral infectious disease. Such pharmaceutical composition comprising the combination means a pharmaceutical composition in which the first antigen-binding molecule and the second antigen-binding molecule are combined such that the first and the second antigen-binding molecules are administered simultaneously, separately, or sequentially to a subject for treating a viral infectious disease. For example, the pharmaceutical composition may be prepared as a mixture or a compounding agent comprising the first antigen-binding molecule and the second antigen-binding molecule together. For another example, a preparation comprising the first antigen-binding molecule and a preparation comprising the second antigen-binding molecule are prepared separately, and these preparations may be used simultaneously or separately.

In one aspect, an antigen-binding molecule capable of binding to a virus that comprise a variant Fc region or polypeptide of the present invention can suppress antibody-dependent enhancement (ADE) observed with conventional anti-virus antibodies. ADE is a phenomenon where a virus bound to an antibody is phagocytosed via activating Fc gamma receptors so that infection of the virus to a cell is enhanced. Fc modifications that reduce interaction with activating Fc gamma receptors could alleviate the risk of ADE. Mutations at positions 234 and 235 from leucine to alanine to form LALA mutants have been shown to reduce the risk of ADE of dengue infection in vivo (Cell Host Microbe (2010) 8, 271-283). Such modifications, however, reduce the other effector immune functions mediated by antibodies, such as ADCC and CDC. Especially, CDC can be expected to play an important role in inhibiting ADE, therefore complement component C1q binding of Fc regions should not be reduced for therapeutic efficacy. As described above, the “one-armed” molecular format of the antigen-binding molecule provided herein can enhance hexamer formation and help maintaining or increasing the binding to complement C1q. One or more mutations at amino acid positions 236, 267, 268, 324, 326, 332, and 333 according to EU numbering also contribute to maintain or increase the activity to bind to C1q. Furthermore, half-life of an antigen-binding molecule can be extended by engineering Fc regions to change binding affinity to its salvage receptor, FcRn, for example, by introducing one or more mutations at amino acid positions 428, 434, 436, 438, and 440 according to EU numbering. Prolonged half-life may lead to prophylactic use of antibodies for protecting a subject from viral infection.

In one embodiment, the virus in the present disclosure is preferably selected from an adenovirus, an astrovirus, a hepadnavirus, a herpesvirus, a papovavirus, a poxvirus, an arenavirus, a bunyavirus, a calcivirus, a coronavirus, a filovirus, a flavivirus, an orthomyxovirus, a paramyxovirus, a picornavirus, a reovirus, a retrovirus, a rhabdovirus, or a togavirus.

In preferred embodiments, the adenovirus includes, but is not limited to, a human adenovirus. In preferred embodiments, the astrovirus includes, but is not limited to, a mamastrovirus. In preferred embodiments, the hepadnavirus includes, but is not limited to, the hepatitis B virus. In preferred embodiments, the herpesvirus includes, but is not limited to, a herpes simplex virus type I, a herpes simplex virus type 2, a human cytomegalovirus, an Epstein-Barr virus, a varicella zoster virus, a roseolovirus, and a Kaposi's sarcoma-associated herpesvirus. In preferred embodiments, the papovavirus includes, but is not limited to, human papilloma virus and a human polyoma virus. In preferred embodiments, the poxvirus includes, but is not limited to, a variola virus, a vaccinia virus, a cowpox virus, a monkeypox virus, a smallpox virus, a pseudocowpox virus, a papular stomatitis virus, a tanapox virus, a yaba monkey tumor virus, and a molluscum contagiosum virus. In preferred embodiments, the arenavirus includes, but is not limited to lymphocytic choriomeningitis virus, a lassa virus, a machupo virus, and a junin virus. In preferred embodiments, the bunyavirus includes, but is not limited to, a hanta virus, a nairovirus, an orthobunyavirus, and a phlebovirus. In preferred embodiments, the calcivirus includes, but is not limited to, a vesivirus, a norovirus, such as the Norwalk virus and a sapovirus. In preferred embodiments, the coronavirus includes, but is not limited to, a human coronavirus (etiologic agent of severe acute respiratory syndrome (SARS)), and severe acute respiratory syndrome coronavirus 2 (SARS-CoV-2). In preferred embodiments, the filovirus includes, but is not limited to, an Ebola virus and a Marburg virus. In preferred embodiments, the flavivirus includes, but is not limited to, a yellow fever virus, a West Nile virus, a dengue virus (DENY-1, DENV-2, DENV-3 and DENV-4), a hepatitis C virus, a tick borne encephalitis virus, a Japanese encephalitis virus, a Murray Valley encephalitis virus, a St. Louis encephalitis virus, a Russian spring-summer encephalitis virus, a Omsk hemorrhagic fever virus, a bovine viral diarrhea virus, a Kyasanus Forest disease virus, and a Powassan encephalitis virus. In preferred embodiments, the orthomyxovirus includes, but is not limited to, influenza virus type A, influenza virus type B, and influenza virus type C. In preferred embodiments, the paramyxovirus includes, but is not limited to, a parainfluenza virus, a rubula virus (mumps), a morbillivirus (measles), a pneumovirus, such as a human respiratory syncytial virus, and a subacute sclerosing panencephalitis virus. In preferred embodiments, the picornavirus includes, but is not limited to, a poliovirus, a rhinovirus, a coxsackievirus A, a coxsackievirus B, a hepatitis A virus, an echovirus, and an eneterovirus. In preferred embodiments, the reovirus includes, but is not limited to, a Colorado tick fever virus and a rotavirus. In preferred embodiments, the retrovirus includes, but is not limited to, a lentivirus, such as a human immunodeficiency virus, and a human T-lymphotrophic virus (HTLV). In preferred embodiments, the rhabdovirus includes, but is not limited to, a lyssavirus, such as the rabies virus, the vesicular stomatitis virus and the infectious hematopoietic necrosis virus. In preferred embodiments, the togavirus includes, but is not limited to, an alphavirus, such as a Ross river virus, an O'nyong'nyong virus, a Sindbis virus, a Venezuelan equine encephalitis virus, an Eastern equine encephalitis virus, and a Western equine encephalitis virus, and a rubella virus.

Pharmaceutical compositions comprising an antigen-binding molecule or antibody as described herein are prepared by mixing such antigen-binding molecule or antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. Such active ingredients are suitably present in combination in amounts that are effective for the purpose intended.

If necessary, the antigen-binding molecules or antibodies of the present disclosure may be encapsulated in microcapsules (microcapsules made from hydroxymethylcellulose, gelatin, poly[methylmethacrylate], and the like), and made into components of colloidal drug delivery systems (liposomes, albumin microspheres, microemulsions, nano-particles, and nano-capsules) (for example, see “Remington's Pharmaceutical Science 16^{th} edition”, Oslo Ed. (1980)). Moreover, methods for preparing agents as sustained-release agents are known, and these can be applied to the antigen-binding molecules of the present disclosure (J. Biomed. Mater. Res. (1981) 15, 267-277; Chemtech. (1982) 12, 98-105; U.S. Pat. No. 3,773,719; European Patent Application (EP) Nos. EP58481 and EP133988; Biopolymers (1983) 22, 547-556).

If necessary, the vectors comprising nucleic acid molecule encodes the antigen-binding molecules of the present disclosure may be introduced to subjects, to express the antigen-binding molecules or antibodies of the present disclosure directly within the subject. An example of vectors that is possible to be used is adenovirus, but not limited to. It is also possible to administer the nucleic acid molecule encodes the antigen-binding molecules or antibodies of the present disclosure directly into a subject, or transfer the nucleic acid molecule encodes the antigen-binding molecules or antibodies of the present disclosure via electroporation to a subject, or administer cells comprises nucleic acid molecule encodes the antigen-binding molecules or antibodies of the present disclosure to be expressed and secreted into a subject, to express and secrete the antigen-binding molecules or antibodies of the present disclosure in the subject continuously.

The pharmaceutical compositions of the present disclosure may be administered either orally or parenterally to patients. Parental administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions, therapeutic agents for inducing cellular cytotoxicity, cell growth-suppressing agents, or anticancer agents of the present disclosure can be administered locally or systemically by injection. Furthermore, appropriate administration methods can be selected according to the patient's age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present disclosure is not limited to these doses.

In one aspect, the present disclosure provides a method for inducing lysis of a target cell or target virus, comprising contacting a target cell or a target virus with the antigen-binding molecule or the pharmaceutical composition of the present disclosure in the presence of complement factors. In some embodiments, the antigen-binding molecule of the present disclosure used in such method comprises an antigen-binding moiety which specifically binds to an antigen on the target cell or the target virus.

In the present disclosure, “contact” can be carried out, for example, by adding an antigen-binding molecule of the present disclosure to culture media of cells/viruses expressing the antigen of interest cultured in vitro. In this case, an antigen-binding molecule to be added can be used in an appropriate form, such as a solution or solid prepared by lyophilization or the like. When the antigen-binding molecule of the present disclosure is added as an aqueous solution, the solution may be a pure aqueous solution containing the antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The added concentration is not particularly limited; however, the final concentration in a culture medium is preferably in a range of 1 pg/ml to 1 g/ml, more preferably 1 ng/ml to 1 mg/ml, and still more preferably 1 micro g/ml to 1 mg/ml.

In another embodiment of the present disclosure, “contact” can also be carried out by administration to animals having cells expressing the antigen of interest or having been infected with a virus expressing the antigen of interest. The administration method may be oral or parenteral. Parenteral administration is particularly preferred. Specifically, the parenteral administration method includes injection, nasal administration, pulmonary administration, and percutaneous administration. Injections include, for example, intravenous injections, intramuscular injections, intraperitoneal injections, and subcutaneous injections. When the antigen-binding molecule is administered as an aqueous solution, the solution may be a pure aqueous solution containing the antigen-binding molecule alone or a solution containing, for example, an above-described surfactant, excipient, coloring agent, flavoring agent, preservative, stabilizer, buffering agent, suspending agent, isotonizing agent, binder, disintegrator, lubricant, fluidity accelerator, and corrigent. The administered dose can be selected, for example, from the range of 0.0001 to 1,000 mg per kg of body weight for each administration. Alternatively, the dose can be selected, for example, from the range of 0.001 to 100,000 mg/body for each patient. However, the dose of an antigen-binding molecule of the present disclosure is not limited to these examples.

The present disclosure also provides kits for use in a method of the present disclosure, which contain an antigen-binding molecule of the present disclosure or an antigen-binding molecule produced by a method of the present disclosure. The kits may be packaged with an additional pharmaceutically acceptable carrier or medium, or instruction manual describing how to use the kits, etc.

In another aspect of the invention, an article of manufacture containing materials useful for the treatment and/or prevention of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating and/or preventing the condition and may have a sterile access port (for example the container may be an intravenous solution bag or a vial having a stopper pierceable by a hypodermic injection needle). At least one active ingredient in the composition is an antibody or an antigen-binding molecule of the present disclosure. The label or package insert indicates that the composition is used for treating the condition of choice. Moreover, the article of manufacture may comprise (a) a first container with a composition contained therein, wherein the composition comprises an antibody or an antigen-binding molecule of the present disclosure; and (b) a second container with a composition contained therein, wherein the composition comprises a further anti-viral, anti-bacterial, or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### Package Inserts

The term “package insert” is used to refer to instructions customarily included in commercial packages of therapeutic products, that contain information about the indications, usage, dosage, administration, combination therapy, contraindications and/or warnings concerning the use of such therapeutic products.

### Pharmaceutical Formulation

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

### Pharmaceutically Acceptable Carrier

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding molecules or antibodies of the present disclosure are used to delay development of a disease or to slow the progression of a disease.

### Other Agents and Treatments

The antigen-binding molecules described herein may be administered in combination with one or more other agents in therapy. For instance, an antigen-binding molecule as described herein may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an anti-viral agent, an anti-bacterial agent, an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of antigen-binding molecules used, the type of disorder or treatment, and other factors discussed above. The antigen-binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the antigen-binding molecules described herein can occur prior to, simultaneously, and/or following, administration of the additional therapeutic agent and/or adjuvant.

All documents cited herein are incorporated herein by reference.

The following are examples of methods and compositions of the present disclosure. It is understood that various other embodiments may be practiced, given the general description provided above.

### EXAMPLES

The following are examples of methods and compositions of the invention. It is understood that various other embodiments may be practiced, given the general description provided above.

### Example 1

### Generation of Antibody CH Variants for Improved Properties

Multiple mutations were introduced to a human IgG1 heavy chain constant region CH and as a result of that, human IgG1 CH variants, SG192 (SEQ ID NO: 1), SG1095 (SEQ ID NO: 2), SG1095R (SEQ ID NO: 3), SG1095RG (SEQ ID NO: 4), SG1095RGY (SEQ ID NO: 5), SG1095ER (SEQ ID NO: 6), and SG1408 (SEQ ID NO: 7), were generated. The genes encoding the CH variants were combined with VH of anti-HER2 antibody (HER2H, SEQ ID NO: 8). The VL gene of the anti-HER2 antibody (HER2L, SEQ ID NO: 9) was combined with a human CL (SK1, SEQ ID NO: 10). Each of them was cloned into an expression vector. The details of the CH variants, i.e., amino acid sequences of the CH and mutations therein identified in EU numbering, are summarized in Table 2. “LALA” stands for the combination of mutations L234A and L235A; “KAES” stands for the combination of mutations K326A and E333S; and “R”, “RG”, “RGY”, and “ER” stands for “E345R”, “E345R and E430G”, “E345R, E430G, and S440Y”, and “K248E and T437R”, respectively.

TABLE 2

| | | | SEQ |
|---|---|---|---|
| Variant name | ID | Mutations | ID NO: |
| LALA | SG192 | L234A, L235A | 1 |
| LALA + KAES | SG1095 | L234A, L235A, K326A, E333S | 2 |
| LALA + KAES + | SG1095R | L234A, L235A, K326A, E333S, E345R | 3 |
| R | | | |
| LALA + KAES + | SG1095RG | L234A, L235A, K326A, E333S, E345R, | 4 |
| RG | | E430G | |
| LALA + KAES + | SG1095RGY | L234A, L235A, K326A, E333S, E345R, | 5 |
| RGY | | E430G, S440Y | |
| LALA + KAES + | SG1095ER | L234A, L235A, K326A, E333S, K248E, | 6 |
| ER | | T437R | |
| LALA + C1q-silent | SG1408 | L234A, L235A, A327G, A330S, P331S | 7 |

### Example 2

### Expression and Purification of Recombinant Antibodies

Recombinant antibodies were expressed transiently using Expi293 cell line (Thermo Fisher, Carlsbad, Calif., USA). Antibody purification was carried out using Protein A affinity chromatography and gel filtration. The combination of genes encoding heavy chains and light chains for each antibody used for co-transfection is summarized in Table 3 and Table 4. For antibody samples with RG (E345R and E430G) or RGY (E345R, E430G and S440Y) mutation on CH region, oligomeric form was fractionated on gel filtration and used for thereafter investigation.

TABLE 3

| Antibody name | VH_{A} | CH_{A} | VL_{A} | CL_{A} |
|---|---|---|---|---|
| Bivalent anti- | HER2H | SG192 | HER2L | SK1 |
| HER2-SG192 | (SEQ ID NO: 8) | (SEQ ID NO: 1) | (SEQ ID NO: 9) | (SEQ ID NO: 10) |
| Bivalent anti- | HER2H | SG1095 | HER2L | SK1 |
| HER2-SG1095 | (SEQ ID NO: 8) | (SEQ ID NO: 2) | (SEQ ID NO: 9) | (SEQ ID NO: 10) |
| Bivalent anti- | HER2H | SG1095R | HER2L | SK1 |
| HER2-SG1095R | (SEQ ID NO: 8) | (SEQ ID NO: 3) | (SEQ ID NO. 9) | (SEQ ID NO: 10) |
| Bivalent anti- | HER2H | SG1095RG | HER2L | SK1 |
| HER2-SG1095RG | (SEQ ID NO: 8) | (SEQ ID NO: 4) | (SEQ ID NO: 9) | (SEQ ID NO: 10) |
| Bivalent anti- | HER2H | SG1095RGY | HER2L | SK1 |
| HER2-SG1095RGY | (SEQ ID NO: 8) | (SEQ ID NO: 5) | (SEQ ID NO: 9) | (SEQ ID NO: 10) |
| Bivalent anti- | HER2H | SG1095ER | HER2L | SK1 |
| HER2-SG1095ER | (SEQ ID NO: 8) | (SEQ ID NO: 6) | (SEQ ID NO: 9) | (SEQ ID NO: 10) |
| Bivalent anti- | HER2H | SG1408 | HER2L | SK1 |
| HER2-SG1408 | (SEQ ID NO: 8) | (SEQ ID NO: 7) | (SEQ ID NO: 9) | (SEQ ID NO: 10) |

TABLE 4

| Antibody name | VH_{A} | CH_{A} (KiH) | VL_{A} | CL_{A} | CH_{B} (KiH) |
|---|---|---|---|---|---|
| One-arm anti- | HER2H | SG192kV11 | HER2L | SK1 | nSG192hV11 |
| HER2-SG192 | (SEQ*: 8) | (SEQ: 17) | (SEQ: 9) | (SEQ: 10) | (SEQ: 24) |
| One-arm anti- | HER2H | SG1095kV11 | HER2L | SK1 | nSG1095hV11 |
| HER2-SG1095 | (SEQ: 8) | (SEQ: 11) | (SEQ: 9) | (SEQ: 10) | (SEQ: 18) |
| One-arm anti- | HER2H | SG1095RkV11 | HER2L | SK1 | nSG1095RhV11 |
| HER2-SG1095R | (SEQ: 8) | (SEQ: 12) | (SEQ: 9) | (SEQ: 10) | (SEQ: 19) |
| One-arm anti- | HER2H | SG1095RGkV11 | HER2L | SK1 | nSG1095RGhV11 |
| HER2-SG1095RG | (SEQ: 8) | (SEQ: 13) | (SEQ: 9) | (SEQ: 10) | (SEQ: 20) |
| One-arm anti- | HER2H | SG1095RGYkV11 | HER2L | SK1 | nSG1095RGYhV11 |
| HER2-SG1095RGY | (SEQ: 8) | (SEQ: 14) | (SEQ: 9) | (SEQ: 10) | (SEQ: 21) |
| One-arm anti- | HER2H | SG1095ERkV11 | HER2L | SK1 | nSG1095ERhV11 |
| HER2-SG1095ER | (SEQ: 8) | (SEQ: 15) | (SEQ: 9) | (SEQ: 10) | (SEQ: 22) |
| One-arm anti- | HER2H | SG1408kV11 | HER2L | SK1 | nSG1408hV11 |
| HER2-SG1408 | (SEQ: 8) | (SEQ: 16) | (SEQ: 9) | (SEQ: 10) | (SEQ: 23) |

*SEQ = SEQ ID NO

### Example 3

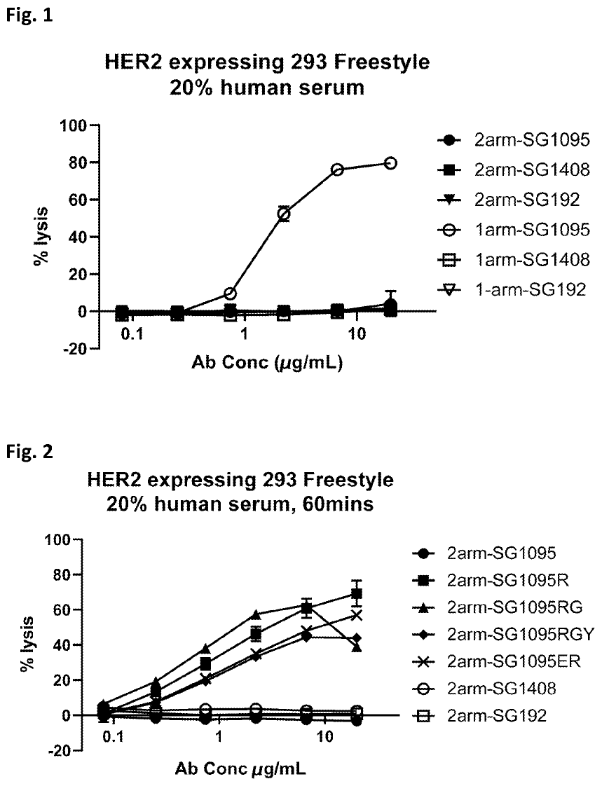

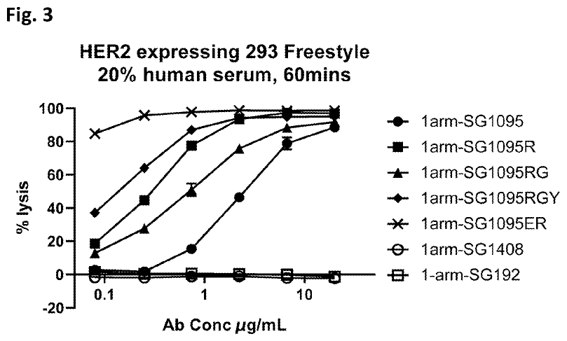

Freestyle 293-F cells (Life Technologies) were stably transfected with a plasmid containing the CAG promoter to overexpress full length HER2 with a C-terminal Histidine tag (SEQ ID: 25). To evaluate the CDC activity of antibodies, the assay was conducted as follows. HER2 overexpressing Freestyle 293-F cells were resuspended at a concentration of approximately 1.25x10⁶ cells/mL using 293 Expression Media (Gibco) and 20 microlitres of cells were seeded into a V-bottom plate. Human serum (Biopredic) was diluted with 293 Expression Media to a working concentration of 40% and 25 microlitres of diluted serum was added to each well. Antibodies to be evaluated were first diluted to 10 times of the final desired concentration with 293 Expression Media, and 5 microlitres of 10 times concentrated antibody was added to each well. This results in a final concentration of 20% human serum and 1 times concentration of antibody in each well. As a negative control, 5 microlitres of 293 expression media was added instead of antibody. As a positive control, 5 microlitres of 1% Triton-X (Biorad) was added instead of antibody. The plate was placed in an orbital shaker for 10 s at 1000 rpm to mix components well, and thereafter placed in a 37 degrees C. incubator with 5% CO₂ for 1 hour. After incubation, the cells were washed once with buffer and stained with 7AAD viability dye (Sigma) for analysis by flow cytometry. To calculate the percentage of cells lysed by antibody-mediated CDC, the negative control containing no antibody was defined as 0% lysis, and positive control containing Triton-X was defined as 100% lysis. Data is representative of 2 experiments. Error bars indicate S. D of duplicate wells. As shown in FIG. 1, the one-armed antigen-binding molecule with the LALA and KAES mutations (1arm-SG1095) showed dose-dependent lysis of HER2 expressing cells, whereas the one-armed antigen-binding molecules having LALA mutation only or the two-armed antigen-binding molecules having LALA mutation or LALA and KAES mutations did not. The introduction of the R, RG, RGY, or ER mutation, which enhances hexamer formation, strengthened the lytic activity of farm-SG1095 to HER2 expressing cells (FIG. 3). Although the two-armed antigen-binding molecule with the LALA ad KAES mutations showed lytic activity to HER2 expressing cells when R, RG, RGY, or ER mutation were further introduced, the activity was lower than that of the corresponding one-armed antigen binding molecules (FIG. 2 and FIG. 3).

### SEQUENCE LISTING

1. An antigen-binding molecule comprising:
(i) a first antigen-binding moiety which specifically binds to an antigen, and
(ii) an Fc polypeptide,
wherein the Fc polypeptide comprises a first Fc region variant and a second Fc region variant each comprising at least one amino acid alteration relative to a parent Fc region, wherein the first Fc region variant is fused to the first antigen-binding moiety, provided that the second Fc region variant is not fused to any other antigen-binding moieties which specifically binds to the antigen, and wherein the antigen-binding molecule has a substantially decreased Fc gamma R-binding activity and has a maintained or increased C1q-binding activity when compared to an antigen binding molecule comprising the parent Fc region.

2. The antigen-binding molecule of claim 1, wherein the antigen-binding molecule further comprises a second antigen-binding moiety which specifically binds to an epitope on the antigen that is different from the epitope on the antigen bound by the first antigen-binding moiety.

3. The antigen-binding molecule of claim 1, wherein the antigen-binding molecule further comprises a second antigen-binding moiety which specifically binds to the same epitope as the one on the antigen bound by the first antigen-binding moiety.

4. The antigen-binding molecule of claim 2 or 3, wherein the second antigen-binding moiety is fused to the N-terminus of the first antigen-binding moiety.

5. The antigen-binding molecule of any one of claims 1 to 4, wherein the first antigen-binding moiety and/or the second antigen-binding moiety comprises a Fab, scFv, VHH, VL, VH, single domain antibody or ligand.

6. The antigen-binding molecule of any one of claims 1 to 5, wherein each of the first Fc region variant and the second Fc region variant comprises Ala at position 234 and Ala at position 235 according to EU numbering.

7. The antigen-binding molecule of claim 6, wherein each of the first Fc region variant and the second Fc region variant comprises further amino acid alterations at positions of any one of the following (a)-(c):
(a) positions 267, 268, and 324;
(b) positions 236, 267, 268, 324, and 332; and
(c) positions 326 and 333;
according to EU numbering.

8. The antigen-binding molecule of claim 7, wherein each of the first Fc region variant and the second Fc region variant comprises amino acids selected from the group consisting of:
(a) Glu at position 267;
(b) Phe at position 268;
(c) Thr at position 324;
(d) Ala at position 236;
(e) Glu at position 332;
(f) Ala, Asp, Glu, Met, or Trp at position 326; and
(g) Ser at position 333;
according to EU numbering.

9. The antigen-binding molecule of any one of claims 1 to 8, wherein each of the first Fc region variant and the second Fc region variant comprises amino acids selected from the group consisting of:
(a) Ala at position 434;
(b) Ala at position 434, Thr at position 436, Arg at position 438, and Glu at position 440;
(c) Leu at position 428, Ala at position 434, Thr at position 436, Arg at position 438, and Glu at position 440;
(d) Leu at position 428, and Ala at position 434; and
(e) Leu at position 428, Ala at position 434, Arg at position 438, and Glu at position 440;
according to EU numbering.

10. The antigen-binding molecule of any one of claims 1 to 9, wherein each of the first Fc region variant and the second Fc region variant comprises at least one amino acid alteration that enhances hexamer formation.

11. The antigen-binding molecule of any one of claims 1 to 10, wherein each of the first Fc region variant and the second Fc region variant comprises at least one amino acid alteration promoting the association of the first Fc region variant and the second Fc region variant.

12. The antigen-binding molecule of any one of claims 1 to 11, which has reduced risk to cause antibody-dependent enhancement (ADE) of entry of a pathogen expressing the antigen into a cell.

13. The antigen-binding molecule of any one of claims 1 to 12, which recruits C1q to eliminate a pathogen or a cell infected by the pathogen via complement-dependent cytotoxicity (CDC).

14. A pharmaceutical composition comprising the antigen-binding molecule of any one of claims 1 to 13, and a pharmaceutically acceptable carrier.

15. An isolated nucleic acid encoding the antigen-binding molecule of any one of claims 1 to 13.


SEQUENCE LISTING (67 sequences)

SEQ ID NO: 1 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 2 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 3 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 4 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSP

SEQ ID NO: 5 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSP

SEQ ID NO: 6 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPEDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYRQKSLSLSP

SEQ ID NO: 7 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 8 - protein, 120 aa, Artificial Sequence [sequence omitted]

SEQ ID NO: 9 - protein, 107 aa, Artificial Sequence [sequence omitted]

SEQ ID NO: 10 - protein, 107 aa, Artificial Sequence [sequence omitted]

SEQ ID NO: 11 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 12 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 13 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVYTLPPCREEMTKNQVSLWCLVKGFYPSDIAVEWESNGQPENNYKTTPPVLDSDGSFFLYSKLTVDKSRWQQGNVFSCSVMHGALHNHYTQKSLSLSP

SEQ ID NO: 14 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVYTLPPCREEMTKNQVSLWCLVKGFYPSDIAVEWESNGQPENNYKTTPPVLDSDGSFFLYSKLTVDKSRWQQGNVFSCSVMHGALHNHYTQKYLSLSP

SEQ ID NO: 15 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPEDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYRQKSLSLSP

SEQ ID NO: 16 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 17 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - protein, 225 aa, Artificial Sequence
  an artificially synthesized sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 225 aa, Artificial Sequence
  an artificially synthesized sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 20 - protein, 225 aa, Artificial Sequence
  an artificially synthesized sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVCTLPPSREEMTKNQVSLSCAVKGFYPSDIAVEWESNGQPENNYKTTPPVLDSDGSFFLVSKLTVDKSRWQQGNVFSCSVMHGALHNHYTQKSLSLSP

SEQ ID NO: 21 - protein, 225 aa, Artificial Sequence
  an artificially synthesized sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPRRPQVCTLPPSREEMTKNQVSLSCAVKGFYPSDIAVEWESNGQPENNYKTTPPVLDSDGSFFLVSKLTVDKSRWQQGNVFSCSVMHGALHNHYTQKYLSLSP

SEQ ID NO: 22 - protein, 225 aa, Artificial Sequence
  an artificially synthesized sequence
DKTHTCPPCPAPEAAGGPSVFLFPPKPEDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYRQKSLSLSP

SEQ ID NO: 23 - protein, 225 aa, Artificial Sequence
  an artificially synthesized sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 24 - protein, 225 aa, Artificial Sequence
  an artificially synthesized sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 25 - protein, 1261 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPVHHHHHH

SEQ ID NO: 26 - protein, 4 aa, Artificial Sequence
  an artificially synthesized sequence
GGGS

SEQ ID NO: 27 - protein, 4 aa, Artificial Sequence
  an artificially synthesized sequence
SGGG

SEQ ID NO: 28 - protein, 5 aa, Artificial Sequence
  an artificially synthesized sequence
GGGGS

SEQ ID NO: 29 - protein, 5 aa, Artificial Sequence
  an artificially synthesized sequence
SGGGG

SEQ ID NO: 30 - protein, 6 aa, Artificial Sequence
  an artificially synthesized sequence
GGGGGS

SEQ ID NO: 31 - protein, 6 aa, Artificial Sequence
  an artificially synthesized sequence
SGGGGG

SEQ ID NO: 32 - protein, 7 aa, Artificial Sequence
  an artificially synthesized sequence
GGGGGGS

SEQ ID NO: 33 - protein, 7 aa, Artificial Sequence
  an artificially synthesized sequence
SGGGGGG

SEQ ID NO: 34 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 35 - protein, 316 aa, Homo sapiens
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 36 - protein, 316 aa, Homo sapiens
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 37 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 38 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLSVSTISSFFPPGYQVSFCLVMVLLFAVDTGLYFSVKTNIRSSTRDWKDHKFKWRKDPQDK

SEQ ID NO: 39 - protein, 254 aa, Homo sapiens [sequence omitted]

SEQ ID NO: 40 - protein, 233 aa, Homo sapiens
MWQLLLPTALLLLVSAGMRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 41 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 42 - protein, 404 aa, Mus musculus
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQS

SEQ ID NO: 43 - protein, 293 aa, Mus musculus
MGILPFLLIPMESNWTVHVFSRTLCHMLLWTAVLNLAAGTHDLPKAVVKLEPPWIQVLKEDTVTLTCEGTHNPGNSSTQWFHNGRSIRSQVQASYTFKATVNDSGEYRCQMEQTRLSDPVDLGVISDWLLLQTPQLVFLEGETITLRCHSWRNKLLNRISFFHNEKSVRYHHYSSNFSIPKANHSHSGDYYCKGSLGRTLHQSKPVTITVQGPKSSRSLPVLTIVAAVTGIAVAAIVIILVSLVYLKKKQVPDNPPDLEEAAKTEAENTITYSLLKHPEALDEETEHDYQNHI

SEQ ID NO: 44 - protein, 267 aa, Mus musculus
MTLDTQMFQNAHSGSQWLLPPLTILLLFAFADRQSAALPKAVVKLDPPWIQVLKEDMVTLMCEGTHNPGNSSTQWFHNWSSIRSQVQSSYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTSSISLVWYHTAFSLVMCLLFAVDTGLYFYVRRNLQTPRDYWRKSLSIRKHQAPQDK

SEQ ID NO: 45 - protein, 249 aa, Mus musculus
MWQLLLPTALVLTAFSGIQAGLQKAVVNLDPKWVRVLEEDSVTLRCQGTFSPEDNSIKWFHNESLIPHQDANYVIQSARVKDSGMYRCQTALSTISDPVQLEVHMGWLLLQTTKWLFQEGDPIHLRCHSWQNRPVRKVTYLQNGKGKKYFHENSELLIPKATHNDSGSYFCRGLIGHNNKSSASFRISLGDPGSPSMFPPWHQITFCLLIGLLFAIDTVLYFSVRRGLQSPVADYEEPKIQWSKEPQDK

SEQ ID NO: 46 - protein, 365 aa, Homo sapiens
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 47 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 48 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 49 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 50 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 51 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 52 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNWALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 53 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 54 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 55 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNDALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 56 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNEALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 57 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNMALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 58 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 59 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 60 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNAALPAPISKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 61 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 62 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 63 - protein, 330 aa, Homo sapiens [sequence omitted]

SEQ ID NO: 64 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 65 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 66 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 328 aa, Artificial Sequence
  an artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP